SEQUENCE LISTING (206 sequences)

SEQ ID NO: 1 - protein, 25 aa, synthetic construct
  REPEAT 1..25: note = This sequence may encompass 1-5 "Gly GLy Gly Gly Ser" repeating units
GGGGSGGGGSGGGGSGGGGSGGGGS

SEQ ID NO: 2 - protein, 4 aa, synthetic construct
GGGS

SEQ ID NO: 3 - protein, 10 aa, synthetic construct
GGGGSGGGGS

SEQ ID NO: 4 - protein, 15 aa, synthetic construct
GGGGSGGGGSGGGGS

SEQ ID NO: 5 - protein, 20 aa, synthetic construct
GGGGSGGGGSGGGGSGGGGS

SEQ ID NO: 6 - protein, 21 aa, synthetic construct
GGGGGSGGGGSGGGGSGGGGS

SEQ ID NO: 7 - protein, 21 aa, synthetic construct
GGGGSGGGGSGGGGSGGGGAS

SEQ ID NO: 8 - protein, 5 aa, synthetic construct
TGEKP

SEQ ID NO: 9 - protein, 4 aa, synthetic construct
GGRR

SEQ ID NO: 10 - protein, 14 aa, synthetic construct
EGKSSGSGSESKVD

SEQ ID NO: 11 - protein, 18 aa, synthetic construct
KESGSVSSEQLAQFRSLD

SEQ ID NO: 12 - protein, 8 aa, synthetic construct
GGRRGGGS

SEQ ID NO: 13 - protein, 9 aa, synthetic construct
LRQRDGERP

SEQ ID NO: 14 - protein, 12 aa, synthetic construct
LRQKDGGGSERP

SEQ ID NO: 15 - protein, 18 aa, synthetic construct
GSTSGSGKPGSGEGSTKG

SEQ ID NO: 16 - protein, 5 aa, synthetic construct
EPKSC

SEQ ID NO: 17 - protein, 10 aa, synthetic construct
EPKSCGGGGS

SEQ ID NO: 18 - protein, 10 aa, synthetic construct
EPKSCDKTHT

SEQ ID NO: 19 - protein, 15 aa, synthetic construct
EPKSCDKTHTGGGGS

SEQ ID NO: 20 - protein, 5 aa, synthetic construct
DKTHT

SEQ ID NO: 21 - protein, 10 aa, synthetic construct
DKTHTGGGGS

SEQ ID NO: 22 - protein, 5 aa, synthetic construct
DGGGS

SEQ ID NO: 23 - protein, 275 aa, synthetic construct
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 24 - protein, 99 aa, synthetic construct [sequence omitted]

SEQ ID NO: 25 - protein, 6 aa, synthetic construct
STYGVS

SEQ ID NO: 26 - protein, 17 aa, synthetic construct
  VARIANT 10: note = X is Y or H
  VARIANT 13: note = X is S or D
  VARIANT 14: note = X is W or S
  VARIANT 16: note = X is N or K
YIDPVYGSTXYAXXVXG

SEQ ID NO: 27 - protein, 15 aa, synthetic construct
  VARIANT 11: note = X is M or V
DLYAGSSGYYXIYSL

SEQ ID NO: 28 - protein, 12 aa, synthetic construct
  VARIANT 1: note = X is Q or R
XSSQSVYNNLLG

SEQ ID NO: 29 - protein, 7 aa, synthetic construct
  VARIANT 5: note = X is L or R
SASTXAS

SEQ ID NO: 30 - protein, 12 aa, synthetic construct
  VARIANT 6: note = X is N or T
QGTYYXGDWYYP

SEQ ID NO: 31 - protein, 6 aa, synthetic construct
STYGVS

SEQ ID NO: 32 - protein, 17 aa, synthetic construct
YIDPVYGSTYYASWVNG

SEQ ID NO: 33 - protein, 15 aa, synthetic construct
DLYAGSSGYYMIYSL

SEQ ID NO: 34 - protein, 12 aa, synthetic construct
QSSQSVYNNLLG

SEQ ID NO: 35 - protein, 7 aa, synthetic construct
SASTLAS

SEQ ID NO: 36 - protein, 12 aa, synthetic construct
QGTYYNGDWYYP

SEQ ID NO: 37 - protein, 6 aa, synthetic construct
STYGVS

SEQ ID NO: 38 - protein, 17 aa, synthetic construct
YIDPVYGSTHYADSVKG

SEQ ID NO: 39 - protein, 15 aa, synthetic construct
DLYAGSSGYYMIYSL

SEQ ID NO: 40 - protein, 12 aa, synthetic construct
RSSQSVYNNLLG

SEQ ID NO: 41 - protein, 7 aa, synthetic construct
SASTLAS

SEQ ID NO: 42 - protein, 12 aa, synthetic construct
QGTYYNGDWYYP

SEQ ID NO: 43 - protein, 6 aa, synthetic construct
STYGVS

SEQ ID NO: 44 - protein, 17 aa, synthetic construct
YIDPVYGSTHYADSVKG

SEQ ID NO: 45 - protein, 15 aa, synthetic construct
DLYAGSSGYYMIYSL

SEQ ID NO: 46 - protein, 12 aa, synthetic construct
RSSQSVYNNLLG

SEQ ID NO: 47 - protein, 7 aa, synthetic construct
SASTLAS

SEQ ID NO: 48 - protein, 12 aa, synthetic construct
QGTYYTGDWYYP

SEQ ID NO: 49 - protein, 6 aa, synthetic construct
STYGVS

SEQ ID NO: 50 - protein, 17 aa, synthetic construct
YIDPVYGSTHYADSVKG

SEQ ID NO: 51 - protein, 15 aa, synthetic construct
DLYAGSSGYYVIYSL

SEQ ID NO: 52 - protein, 12 aa, synthetic construct
RSSQSVYNNLLG

SEQ ID NO: 53 - protein, 7 aa, synthetic construct
SASTLAS

SEQ ID NO: 54 - protein, 12 aa, synthetic construct
QGTYYTGDWYYP

SEQ ID NO: 55 - protein, 6 aa, synthetic construct
STYGVS

SEQ ID NO: 56 - protein, 17 aa, synthetic construct
YIDPVYGSTHYADSVKG

SEQ ID NO: 57 - protein, 15 aa, synthetic construct
DLYAGSSGYYMIYSL

SEQ ID NO: 58 - protein, 12 aa, synthetic construct
RSSQSVYNNLLG

SEQ ID NO: 59 - protein, 7 aa, synthetic construct
SASTRAS

SEQ ID NO: 60 - protein, 12 aa, synthetic construct
QGTYYTGDWYYP

SEQ ID NO: 61 - protein, 29 aa, synthetic construct
EVQLVESGGGSVQPGGSLRLSCAASGFDF

SEQ ID NO: 62 - protein, 14 aa, synthetic construct
WVRQAPGKCLEWIG

SEQ ID NO: 63 - protein, 32 aa, synthetic construct
RFTISRDNSKNTLYLQMNSLRAEDTASYYCVR

SEQ ID NO: 64 - protein, 11 aa, synthetic construct
WGQGTSVTVSS

SEQ ID NO: 65 - protein, 23 aa, synthetic construct
DIQMTQSPSSLSASVGDRVTITC

SEQ ID NO: 66 - protein, 15 aa, synthetic construct
WYQQKPGKAPKLLIY

SEQ ID NO: 67 - protein, 32 aa, synthetic construct
GVPSRFSGSGSGTDFTLTISSLQPEDFATYYC

SEQ ID NO: 68 - protein, 10 aa, synthetic construct
FGCGTKVEIK

SEQ ID NO: 69 - protein, 29 aa, synthetic construct
QVQLVESGGGSVQPGRSLRLSCAASGFDF

SEQ ID NO: 70 - protein, 32 aa, synthetic construct
GVPSRFSGSGSGTDFTLTISSLQPEDSATYYC

SEQ ID NO: 71 - protein, 124 aa, synthetic construct
QEQLVESGGGLVQPGGSLKLSCKASGFDFSTYGVSWVRQAPGKGLEWIGYIDPVYGSTYYASWVNGRFTISSHNAQNTLYLQLNSLTAADTATYFCVRDLYAGSSGYYMIYSLWGPGTLVTVSS

SEQ ID NO: 72 - protein, 111 aa, synthetic construct
ELVMTQTPSSVSAAVGGTVTIKCQSSQSVYNNLLGWYQQKPGQPPKLLIYSASTLASGVPSRFSGSGSGTEFTLTISGVQADDAASYYCQGTYYNGDWYYPFGGGTEVVVK

SEQ ID NO: 73 - protein, 124 aa, synthetic construct
EVQLVESGGGSVQPGGSLRLSCAASGFDFSTYGVSWVRQAPGKCLEWIGYIDPVYGSTYYASWVNGRFTISRDNSKNTLYLQMNSLRAEDTASYYCVRDLYAGSSGYYMIYSLWGQGTSVTVSS

SEQ ID NO: 74 - protein, 111 aa, synthetic construct
DIQMTQSPSSLSASVGDRVTITCQSSQSVYNNLLGWYQQKPGKAPKLLIYSASTLASGVPSRFSGSGSGTDFTLTISSLQPEDFATYYCQGTYYNGDWYYPFGCGTKVEIK

SEQ ID NO: 75 - protein, 124 aa, synthetic construct
QVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 76 - protein, 111 aa, synthetic construct
DIQMTQSPSSLSASVGDRVTITCRSSQSVYNNLLGWYQQKPGKAPKLLIYSASTLASGVPSRFSGSGSGTDFTLTISSLQPEDFATYYCQGTYYNGDWYYPFGCGTKVEIK

SEQ ID NO: 77 - protein, 124 aa, synthetic construct; identical to SEQ ID NO: 75

SEQ ID NO: 78 - protein, 111 aa, synthetic construct
DIQMTQSPSSLSASVGDRVTITCRSSQSVYNNLLGWYQQKPGKAPKLLIYSASTLASGVPSRFSGSGSGTDFTLTISSLQPEDFATYYCQGTYYTGDWYYPFGCGTKVEIK

SEQ ID NO: 79 - protein, 124 aa, synthetic construct; identical to SEQ ID NO: 75

SEQ ID NO: 80 - protein, 111 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIK

SEQ ID NO: 81 - protein, 124 aa, synthetic construct
QVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 82 - protein, 111 aa, synthetic construct; identical to SEQ ID NO: 80

SEQ ID NO: 83 - protein, 124 aa, synthetic construct
QVQLVESGGGSVQPGRSLRLSCAASGFTVSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 84 - protein, 111 aa, synthetic construct; identical to SEQ ID NO: 80

SEQ ID NO: 85 - protein, 124 aa, synthetic construct
QVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 86 - protein, 111 aa, synthetic construct; identical to SEQ ID NO: 80

SEQ ID NO: 87 - protein, 113 aa, synthetic construct
VQPGRSLRLSCAASGFTFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 88 - protein, 111 aa, synthetic construct; identical to SEQ ID NO: 80

SEQ ID NO: 89 - protein, 124 aa, synthetic construct
QVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYVIYSLWGQGTSVTVSS

SEQ ID NO: 90 - protein, 111 aa, synthetic construct; identical to SEQ ID NO: 80

SEQ ID NO: 91 - protein, 124 aa, synthetic construct; identical to SEQ ID NO: 75

SEQ ID NO: 92 - protein, 111 aa, synthetic construct
DIQMTQSPSSLSASVGDRVTITCRSSQSVYNNLLGWYQQKPGKAPKLLIYSASTRASGVPSRFSGSGSGTDFTLTISSLQPEDSATYYCQGTYYTGDWYYPFGCGTKVEIK

SEQ ID NO: 93 - protein, 255 aa, synthetic construct
QEQLVESGGGLVQPGGSLKLSCKASGFDFSTYGVSWVRQAPGKGLEWIGYIDPVYGSTYYASWVNGRFTISSHNAQNTLYLQLNSLTAADTATYFCVRDLYAGSSGYYMIYSLWGPGTLVTVSSGGGGSGGGGSGGGGSGGGGSELVMTQTPSSVSAAVGGTVTIKCQSSQSVYNNLLGWYQQKPGQPPKLLIYSASTLASGVPSRFSGSGSGTEFTLTISGVQADDAASYYCQGTYYNGDWYYPFGGGTEVVVK

SEQ ID NO: 94 - protein, 255 aa, synthetic construct
DIQMTQSPSSLSASVGDRVTITCQSSQSVYNNLLGWYQQKPGKAPKLLIYSASTLASGVPSRFSGSGSGTDFTLTISSLQPEDFATYYCQGTYYNGDWYYPFGCGTKVEIKGGGGSGGGGSGGGGSGGGGSEVQLVESGGGSVQPGGSLRLSCAASGFDFSTYGVSWVRQAPGKCLEWIGYIDPVYGSTYYASWVNGRFTISRDNSKNTLYLQMNSLRAEDTASYYCVRDLYAGSSGYYMIYSLWGQGTSVTVSS

SEQ ID NO: 95 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDFATYYCQGTYYNGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 96 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDFATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 97 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 98 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 99 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTVSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 100 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 101 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 102 - protein, 255 aa, synthetic construct
DIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYVIYSLWGQGTSVTVSS

SEQ ID NO: 103 - protein, 255 aa, synthetic construct
DIQMTQSPSSLSASVGDRVTITCRSSQSVYNNLLGWYQQKPGKAPKLLIYSASTRASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 104 - protein, 228 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 105 - protein, 215 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 106 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQEQLVESGGGLVQPGGSLKLSCKASGFDFSTYGVSWVRQAPGKGLEWIGYIDPVYGSTYYASWVNGRFTISSHNAQNTLYLQLNSLTAADTATYFCVRDLYAGSSGYYMIYSLWGPGTLVTVSSGGGGSGGGGSGGGGSGGGGSELVMTQTPSSVSAAVGGTVTIKCQSSQSVYNNLLGWYQQKPGQPPKLLIYSASTLASGVPSRFSGSGSGTEFTLTISGVQADDAASYYCQGTYYNGDWYYPFGGGTEVVVK

SEQ ID NO: 107 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCQSSQSVYNNLLGWYQQKPGKAPKLLIYSASTLASGVPSRFSGSGSGTDFTLTISSLQPEDFATYYCQGTYYNGDWYYPFGCGTKVEIKGGGGSGGGGSGGGGSGGGGSEVQLVESGGGSVQPGGSLRLSCAASGFDFSTYGVSWVRQAPGKCLEWIGYIDPVYGSTYYASWVNGRFTISRDNSKNTLYLQMNSLRAEDTASYYCVRDLYAGSSGYYMIYSLWGQGTSVTVSS

SEQ ID NO: 108 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDFATYYCQGTYYNGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 109 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDFATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 110 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 111 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 112 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTVSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 113 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 114 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 115 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYVIYSLWGQGTSVTVSS

SEQ ID NO: 116 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSSLSASVGDRVTITCRSSQSVYNNLLGWYQQKPGKAPKLLIYSASTRASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 117 - protein, 488 aa, synthetic construct; identical to SEQ ID NO: 106

SEQ ID NO: 118 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSQEQLVESGGGLVQPGGSLKLSCKASGFDFSTYGVSWVRQAPGKGLEWIGYIDPVYGSTYYASWVNGRFTISSHNAQNTLYLQLNSLTAADTATYFCVRDLYAGSSGYYMIYSLWGPGTLVTVSSGGGGSGGGGSGGGGSGGGGSELVMTQTPSSVSAAVGGTVTIKCQSSQSVYNNLLGWYQQKPGQPPKLLIYSASTLASGVPSRFSGSGSGTEFTLTISGVQADDAASYYCQGTYYNGDWYYPFGGGTEVVVK

SEQ ID NO: 119 - protein, 488 aa, synthetic construct; identical to SEQ ID NO: 107

SEQ ID NO: 120 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCQSSQSVYNNLLGWYQQKPGKAPKLLIYSASTLASGVPSRFSGSGSGTDFTLTISSLQPEDFATYYCQGTYYNGDWYYPFGCGTKVEIKGGGGSGGGGSGGGGSGGGGSEVQLVESGGGSVQPGGSLRLSCAASGFDFSTYGVSWVRQAPGKCLEWIGYIDPVYGSTYYASWVNGRFTISRDNSKNTLYLQMNSLRAEDTASYYCVRDLYAGSSGYYMIYSLWGQGTSVTVSS

SEQ ID NO: 121 - protein, 488 aa, synthetic construct; identical to SEQ ID NO: 108

SEQ ID NO: 122 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDFATYYCQGTYYNGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 123 - protein, 488 aa, synthetic construct; identical to SEQ ID NO: 109

SEQ ID NO: 124 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDFATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 125 - protein, 488 aa, synthetic construct; identical to SEQ ID NO: 110

SEQ ID NO: 126 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 127 - protein, 488 aa, synthetic construct; identical to SEQ ID NO: 111

SEQ ID NO: 128 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 129 - protein, 488 aa, synthetic construct; identical to SEQ ID NO: 112

SEQ ID NO: 130 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTVSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 131 - protein, 488 aa, synthetic construct; identical to SEQ ID NO: 113

SEQ ID NO: 132 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 133 - protein, 488 aa, synthetic construct; identical to SEQ ID NO: 114

SEQ ID NO: 134 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFTFSTYGVSWVRQAPGKCLEWIGYIDPVYGSTHYADSVKGRFTISRDNSKNTLYLQMNSLRAEDTASYYCARDLYAGSSGYYMIYSLWGQGTSVTVSS

SEQ ID NO: 135 - protein, 488 aa, synthetic construct; identical to SEQ ID NO: 115

SEQ ID NO: 136 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTLASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYVIYSLWGQGTSVTVSS

SEQ ID NO: 137 - protein, 488 aa, synthetic construct; identical to SEQ ID NO: 116

SEQ ID NO: 138 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSSLSASVGDRVTITCRSSQSVYNNLLGWYQQKPGKAPKLLIYSASTRASGVPSRFSGSGSGTDFTLTISSLQPEDSATYYCQGTYYTGDWYYPFGCGTKVEIKGGGGSGGGGSGGGGSGGGGSQVQLVESGGGSVQPGRSLRLSCAASGFDFSTYGVSWVRQAPGKCLEWIGYIDPVYGSTHYADSVKGRFTISRDNSKNTLYLQMNSLRAEDTASYYCVRDLYAGSSGYYMIYSLWGQGTSVTVSS

SEQ ID NO: 139 - protein, 9 aa, synthetic construct
SLLQHLIGL

SEQ ID NO: 140 - protein, 6 aa, synthetic construct
  VARIANT 1: note = X is R or S
STYAMN

SEQ ID NO: 141 - protein, 19 aa, synthetic construct
RIRSKYNNYATYYADSVKG

SEQ ID NO: 142 - protein, 14 aa, synthetic construct
HGNFGDSYVSWFAY

SEQ ID NO: 143 - protein, 14 aa, synthetic construct
GSSTGAVTTSNYAN

SEQ ID NO: 144 - protein, 7 aa, synthetic construct
GTNKRAP

SEQ ID NO: 145 - protein, 9 aa, synthetic construct
ALWYSNHWV

SEQ ID NO: 146 - protein, 124 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVS

SEQ ID NO: 147 - protein, 110 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLG

SEQ ID NO: 148 - protein, 9 aa, synthetic construct
GIDDLHISL

SEQ ID NO: 149 - protein, 9 aa, synthetic construct
GLDGHSHLV

SEQ ID NO: 150 - protein, 9 aa, synthetic construct
GLDSYLPEL

SEQ ID NO: 151 - protein, 9 aa, synthetic construct
GLYEGEFTL

SEQ ID NO: 152 - protein, 9 aa, synthetic construct
GQYELVHTL

SEQ ID NO: 153 - protein, 9 aa, synthetic construct
GMTELYFQL

SEQ ID NO: 154 - protein, 9 aa, synthetic construct
GIADLAHLL

SEQ ID NO: 155 - protein, 9 aa, synthetic construct
GLYSKMNGL

SEQ ID NO: 156 - protein, 9 aa, synthetic construct
GVIDAYMTL

SEQ ID NO: 157 - protein, 10 aa, synthetic construct
GVYDGEEHSV

SEQ ID NO: 158 - protein, 10 aa, synthetic construct
SLYDSKIWTT

SEQ ID NO: 159 - protein, 10 aa, synthetic construct
GQYEGHEAEL

SEQ ID NO: 160 - protein, 10 aa, synthetic construct
AQYDGKGVGL

SEQ ID NO: 161 - protein, 9 aa, synthetic construct
ALDVYNGLL

SEQ ID NO: 162 - protein, 9 aa, synthetic construct
GVRGRVEEI

SEQ ID NO: 163 - protein, 9 aa, synthetic construct
YVLDHLIVV

SEQ ID NO: 164 - protein, 9 aa, synthetic construct
GLLSLEEEL

SEQ ID NO: 165 - protein, 9 aa, synthetic construct
LLLGPLGPL

SEQ ID NO: 166 - protein, 9 aa, synthetic construct
VLEETSVML

SEQ ID NO: 167 - protein, 9 aa, synthetic construct
VLFGLGFAI

SEQ ID NO: 168 - protein, 9 aa, synthetic construct
HLVEALYLV

SEQ ID NO: 169 - protein, 9 aa, synthetic construct
SLLFLLFSL

SEQ ID NO: 170 - protein, 9 aa, synthetic construct
SLLMWITQV

SEQ ID NO: 171 - protein, 9 aa, synthetic construct
VLAGGFFLL

SEQ ID NO: 172 - protein, 9 aa, synthetic construct
YLIELIDRV

SEQ ID NO: 173 - protein, 9 aa, synthetic construct
YMDGTMSQV

SEQ ID NO: 174 - protein, 9 aa, synthetic construct
GLADQLIHL

SEQ ID NO: 175 - protein, 9 aa, synthetic construct
LIDQYLYYL

SEQ ID NO: 176 - protein, 9 aa, synthetic construct
ALVEMGHHA

SEQ ID NO: 177 - protein, 9 aa, synthetic construct
SLGEQQYSV

SEQ ID NO: 178 - protein, 9 aa, synthetic construct
VLDFAPPGA

SEQ ID NO: 179 - protein, 10 aa, synthetic construct
YLNDHLEPWI

SEQ ID NO: 180 - protein, 10 aa, synthetic construct
SLYNTVATLY

SEQ ID NO: 181 - protein, 10 aa, synthetic construct
ALWPWLLMAT

SEQ ID NO: 182 - protein, 10 aa, synthetic construct
FLPSPLFFFL

SEQ ID NO: 183 - protein, 10 aa, synthetic construct
ALLTSRLRFI

SEQ ID NO: 184 - protein, 10 aa, synthetic construct
YLQVNSLQTV

SEQ ID NO: 185 - protein, 10 aa, synthetic construct
ALYTGRLPEA

SEQ ID NO: 186 - protein, 9 aa, synthetic construct
SLLQHVLLL

SEQ ID NO: 187 - protein, 9 aa, synthetic construct
SLLKNLIYL

SEQ ID NO: 188 - protein, 9 aa, synthetic construct
SLIQHLEEI

SEQ ID NO: 189 - protein, 9 aa, synthetic construct
SLSQELVGV

SEQ ID NO: 190 - protein, 9 aa, synthetic construct
VLLHHQIGL

SEQ ID NO: 191 - protein, 9 aa, synthetic construct
SLLQKQIML

SEQ ID NO: 192 - protein, 9 aa, synthetic construct
SVLGALIGV

SEQ ID NO: 193 - protein, 9 aa, synthetic construct
SILQTLILV

SEQ ID NO: 194 - protein, 201 aa, synthetic construct
GDAKTTQPNSMESNEEEPVHLPCNHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 195 - protein, 502 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 196 - protein, 467 aa, synthetic construct
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 197 - protein, 454 aa, synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 198 - protein, 9 aa, synthetic construct
KLLKNLIGI

SEQ ID NO: 199 - protein, 9 aa, synthetic construct
KLARHFIGL

SEQ ID NO: 200 - protein, 9 aa, synthetic construct
YLQQHVAGL

SEQ ID NO: 201 - protein, 9 aa, synthetic construct
YMNNDLIGL

SEQ ID NO: 202 - protein, 9 aa, synthetic construct
SLIRRIIGL

SEQ ID NO: 203 - protein, 9 aa, synthetic construct
NLAEKLIGV

SEQ ID NO: 204 - protein, 29 aa, synthetic construct
QVQLVESGGGSVQPGRSLRLSCAASGFTF

SEQ ID NO: 205 - protein, 29 aa, synthetic construct
QVQLVESGGGSVQPGRSLRLSCAASGFTV

SEQ ID NO: 206 - protein, 32 aa, synthetic construct
RFTISRDNSKNTLYLQMNSLRAEDTASYYCAR

(19) United States
(12) Patent Application Publication
(10) Pub. No.: US 2025/0333514 A1
(43) Pub. Date: Oct. 30, 2025

(54) ANTIGEN BINDING PROTEINS TARGETING AN HLA-RESTRICTED PRAME PEPTIDE

(71) Applicant: CDR-LIFE AG, Horgen (CH)

(72) Inventors: Fabian Bert Scheifele, Mägenwil-Aargau (CH); Anna Maria Sobieraj, Zürich (CH); Alessio Vantellini, Wädenswil (CH); Romina Dörig, Zürich (CH); Christian Valdemar Vinge Leisner, Thalwil (CH)

(21) Appl. No.: 19/175,214

(22) Filed: Apr. 10, 2025

Related U.S. Application Data
(60) Provisional application No. 63/632,592, filed on Apr. 11, 2024.
(60) Provisional application No. 63/690,884, filed on Sep. 5, 2024.

(51) Int. Cl. C07K 16/28

(52) U.S. Cl. CPC C07K 16/2833; C07K 16/2809; C07K 2317/24; C07K 2317/31; C07K 2317/622; C07K 2317/92

(57) ABSTRACT

Described herein are antigen binding proteins targeting PRAME derived peptide-MHCs (pMHCs). Also described are multispecific antigen binding proteins comprising an antigen binding domain with specificity to CD3, and at least one target peptide binding domain, in particular bispecific antigen binding proteins targeting both CD3 and PRAME. Methods of treatment with the same are also described.

### RELATED APPLICATIONS

This application claims the benefit of U.S. Provisional Application Ser. No. 63/632,592, filed Apr. 11, 2024, and U.S. Provisional Application Ser. No. 63/690,884, filed Sep. 5, 2024, the entire disclosures of each are hereby incorporated herein by reference.

### SEQUENCE LISTING

The instant application contains a Sequence Listing which has been submitted electronically in XML format and is hereby incorporated by reference in its entirety. Said XML file, created on Apr. 10, 2025, is named 761944_CDR9-015_ST26.xml and is 207,025 bytes in size.

### FIELD OF THE DISCLOSURE

This disclosure relates to novel antigen binding proteins that bind to tumor peptide-MHC (pMHC) complexes with high specificity, having favorable properties for therapeutic purposes. Such pMHC binding proteins may be incorporated into CARs, an ADC or further comprise a CD3 targeting moiety which provide efficient T-cell mediated cancer cell killing despite very low levels of pMHC on the cell surface.

### BACKGROUND

PRAME is a clinically validated cancer specific target and both T cell engagers (TCE) and TCR-T cell therapies confirmed good responses in heavily pre-treated patients. Importantly, PRAME is a pan cancer target, antigen presentation is observed in tumors only, with the exception of kidneys that express low levels of PRAME. The antigen is highly prevalent in solid tumors. The PRAME peptide SLLQHLIGL (SEQ ID NO: 139) is displayed on major histocompatibility complexes (MHCs) in a variety of cancer cell types, including those of high unmet medical need. It is therefore an attractive target for therapeutic antigen binding proteins.

Peptide-MHC complexes (pMHCs) derived from intracellular tumor associated antigens (TAAs), such as SLLQHLIGL (SEQ ID NO: 139), represent a large repertoire of novel targets for immunotherapy. They have been traditionally targeted by TCR-engineered T cells or soluble recombinant T-cell receptors (TCRs) fused to an anti-CD3 fragment. However, therapeutic use of soluble TCRs is hampered by low target affinity and challenges related to expression. Naturally occurring cancer reactive TCRs typically exhibit low binding affinities for their pMHC targets. Therefore, substantial engineering efforts are needed to achieve better affinity as well as better biophysical properties to be developed as drugs which may compromise the required specificity to the pMHC target. Conversely, antibodies may exhibit affinities in the nanomolar range or even below. However, artificial antibodies do not pass through thymic selection which may dampen putative cross-reactivity of naturally occurring TCRs; hence, there remains an inherent challenge that TCR-like antibodies may cross-react with pMHCs presenting similar peptides, leading to undesired side-effects. Another obstacle to the development of immunotherapies involving TCRs or TCR-like antibodies is the low population coverage as the high level of polymorphism of the HLA genes results in a highly diverse number of pMHCs.

### SUMMARY

The present invention relates to antigen binding proteins which specifically bind to a major histocompatibility complex (MHC)-displayed PRAME peptide SLLQHLIGL (SEQ ID NO: 139).

The antigen binding proteins of the disclosure exhibit advantageous effector functions which make them suitable for therapeutic off the shelf application on HLA-A*02:01 patients cancer patients. These include, inter alia, high affinity and avidity binding and an excellent in vitro safety profile. Furthermore, bispecific antigen binding proteins of the disclosure show potent and selective PRAME-specific tumor cell killing in vitro, an IgG-like predicted PK for optimal dosing schedule. The humanized antigen binding proteins are optimized for low immunogenicity and stability; accordingly, they show good manufacturability. In certain embodiments and as shown in the examples, antigen binding proteins of the disclosure are more potent, less toxic and more specific than comparator molecules; they show a better on-target/off-tumor reactivity profile a broader therapeutic window as well as a larger therapeutic index towards normal primary renal cells. Furthermore, the antigen binding proteins of the disclosure are potent in both medium and high PRAME copy number tumor types.

In one aspect, the disclosure provides an antigen binding protein which specifically binds to a major histocompatibility complex (MHC)-displayed SLLQHLIGL (SEQ ID NO: 139), wherein the antigen binding protein comprises:
(a) an antibody heavy chain variable (VH) domain comprising an HCDR1 amino acid sequence of STYGVS (SEQ ID NO: 25), an HCDR2 amino acid sequence of YIDPVYGSTX₁YAX₂×₃VX₄G (SEQ ID NO: 26), wherein X₁ is Yor H, X₂ is S or D, X₃ is W or S, and X₄ is N or K, and an HCDR3 amino acid sequence of DLYAGSSGYYXIYSL (SEQ ID NO: 27), wherein X₅ is M or V; and
(b) an antibody light chain variable (VL) domain comprising an LCDR1 amino acid sequence of X₆SSQSVYNNLLG (SEQ ID NO: 28), wherein X₆ is Q or R, an LCDR2 amino acid sequence of SASTX₇AS (SEQ ID NO: 29), wherein X₇ is L or R, and an LCDR3 amino acid sequence of QGTYYX₈GDWYYP (SEQ ID NO: 30), wherein X₈ is N or T.

In certain embodiments, said HCDR2 sequence is YIDPVYGSTYYASWVNG (SEQ ID NO: 32) or YIDPVYGSTHYADSVKG (SEQ ID NO: 38).

In certain embodiments, said HCDR3 sequence is DLYAGSSGYYMIYSL (SEQ ID NO: 33) or DLYAGSSGYYVIYSL (SEQ ID NO: 51).

In certain embodiments, said LCDR1 sequence is QSSQSVYNNLLG (SEQ ID NO: 34) or RSSQSVYNNLLG (SEQ ID NO: 46).

In certain embodiments, said LCDR2 sequence is SASTLAS (SEQ ID NO: 41) or SASTRAS (SEQ ID NO: 59).

In certain embodiments, said LCDR3 sequence is QGTYYNGDWYYP (SEQ ID NO: 36) or QGTYYTGDWYYP (SEQ ID NO: 48).

In certain embodiments, the target protein SLLQHLIGL (SEQ ID NO: 139) is displayed by a MHC is of HLA supertype A*02, in particular HLA-A*02:01.

In certain embodiments, the antigen binding protein has an affinity (KD) to an MHC-displayed SLLQHLIGL (SEQ ID NO: 139) of at least about 5 nM or stronger, such as at least 40-160 pM, e.g., about 80 pM.

In certain embodiments, an antigen binding protein of the disclosure comprises:
(a1) an antibody heavy chain variable (VH) domain comprising an HCDR1 amino acid sequence of STYGVS (SEQ ID NO: 37), an HCDR2 amino acid sequence of YIDPVYGSTHYADSVKG (SEQ ID NO: 38), and an HCDR3 amino acid sequence of DLYAGSSGYYMIYSL (SEQ ID NO: 39); and (b1) an antibody light chain variable (VL) domain comprising an LCDR1 amino acid sequence of RSSQSVYNNLLG (SEQ ID NO: 40), an LCDR2 amino acid sequence of SASTLAS (SEQ ID NO: 41), and an LCDR3 amino acid sequence of QGTYYNGDWYYP (SEQ ID NO: 42);
(a2) an antibody heavy chain variable (VH) domain comprising an HCDR1 amino acid sequence of STYGVS (SEQ ID NO: 31), an HCDR2 amino acid sequence of YIDPVYGSTYYASWVNG (SEQ ID NO: 32), and an HCDR3 amino acid sequence of DLYAGSSGYYMIYSL (SEQ ID NO: 33); and (b2) an antibody light chain variable (VL) domain comprising an LCDR1 amino acid sequence of QSSQSVYNNLLG (SEQ ID NO: 34), an LCDR2 amino acid sequence of SASTLAS (SEQ ID NO: 35), and an LCDR3 amino acid sequence of QGTYYNGDWYYP (SEQ ID NO: 36);
(a3) an antibody heavy chain variable (VH) domain comprising an HCDR1 amino acid sequence of STYGVS (SEQ ID NO: 43), an HCDR2 amino acid sequence of YIDPVYGSTHYADSVKG (SEQ ID NO: 44), and an HCDR3 amino acid sequence of DLYAGSSGYYMIYSL (SEQ ID NO: 45); and (b3) an antibody light chain variable (VL) domain comprising an LCDR1 amino acid sequence of RSSQSVYNNLLG (SEQ ID NO: 46), an LCDR2 amino acid sequence of SASTLAS (SEQ ID NO: 47), and an LCDR3 amino acid sequence of QGTYYTGDWYYP (SEQ ID NO: 48);
(a4) an antibody heavy chain variable (VH) domain comprising an HCDR1 amino acid sequence of STYGVS (SEQ ID NO: 49), an HCDR2 amino acid sequence of YIDPVYGSTHYADSVKG (SEQ ID NO: 50), and an HCDR3 amino acid sequence of DLYAGSSGYYVIYSL (SEQ ID NO: 51); and (b4) an antibody light chain variable (VL) domain comprising an LCDR1 amino acid sequence of RSSQSVYNNLLG (SEQ ID NO: 52), an LCDR2 amino acid sequence of SASTLAS (SEQ ID NO: 53), and an LCDR3 amino acid sequence of QGTYYTGDWYYP (SEQ ID NO: 54); or
(a5) an antibody heavy chain variable (VH) domain comprising an HCDR1 amino acid sequence of STYGVS (SEQ ID NO: 55), an HCDR2 amino acid sequence of YIDPVYGSTHYADSVKG (SEQ ID NO: 56), and an HCDR3 amino acid sequence of DLYAGSSGYYMIYSL (SEQ ID NO: 57); and (b5) an antibody light chain variable (VL) domain comprising an LCDR1 amino acid sequence of RSSQSVYNNLLG (SEQ ID NO: 58), an LCDR2 amino acid sequence of SASTRAS (SEQ ID NO: 59), and an LCDR3 amino acid sequence of QGTYYTGDWYYP (SEQ ID NO: 60).

In certain embodiments, an antigen binding protein targeting MHC-displayed SLLQHLIGL (SEQ ID NO: 139) is provided, comprising:
(1) an antibody heavy chain variable (VH) domain comprising the amino acid sequence of SEQ ID NO: 71 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 72 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 31, 32 and 33, and the VL comprises the LCDR1-3 sequences of SEQ ID NOs.: 34, 35 and 36;
(2) an antibody heavy chain variable (VH) domain comprising the amino acid sequence of SEQ ID NO: 73 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 74 or a variant thereof that is at least 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 31, 32 and 33, and the VL comprises the LCDR1-3 sequences of SEQ ID NOs.: 34, 35 and 36;
(3) an antibody heavy chain variable (VH) domain comprising the amino acid sequence of SEQ ID NO: 75 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 76 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 37, 38 and 39, and the VL comprises the LCDR1-3 sequences of SEQ ID NOs.: 40, 41 and 42;
(4) an antibody heavy chain variable (VH) domain comprising the amino acid sequence of SEQ ID NO: 77 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 78 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 43, 44, and 45, and the VL comprises the LCDR1-3 sequences of SEQ ID NOs.: 46, 47 and 48;
(5) an antibody heavy chain variable (VH) domain comprising the amino acid sequence of SEQ ID NO: 79 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 80 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 43, 44, and 45, and the VL comprises the LCDR1-3 sequences of SEQ ID NOs.: 46, 47 and 48;
(6) an antibody heavy chain variable (VH) domain comprising the amino acid sequence of SEQ ID NO: 81 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 82 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 43, 44, and 45, and the VL comprises the LCDR1-3 sequences of SEQ ID NOs.: 46, 47 and 48;
(7) an antibody heavy chain variable (VH) domain comprising the amino acid sequence of SEQ ID NO: 83 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 84 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 43, 44, and 45, and the VL comprises the LCDR1-3 sequences of SEQ ID NOs.: 46, 47 and 48;
(8) an antibody heavy chain variable (VH) domain comprising the amino acid sequence of SEQ ID NO: 85 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 86 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 43, 44, and 45, and the VL comprises the LCDR1-3 sequences of SEQ ID NOs.: 46, 47 and 48;
(9) an antibody heavy chain variable (VH) domain comprising the amino acid sequence of SEQ ID NO: 87 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 88 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 43, 44, and 45, and the VL comprises the LCDR1-3 sequences of SEQ ID NOs.: 46, 47 and 48;
(10) an antibody heavy chain variable (VH) domain comprising the amino acid sequence of SEQ ID NO: 89 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 90 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 49, 50 and 51, and the VL comprises the LCDR1-3 sequences of SEQ ID NOs.: 52, 53 and 54; or
(11) an antibody heavy chain variable (VH) domain comprising the amino acid sequence of SEQ ID NO: 91 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 92 or a variant thereof that is at least about 75%, 78%, 79%, 80%, 81%, 83%, 85%, 90%, 95%, 96%, 97%, 98% or 99% identical thereto, wherein the VH comprises the HCDR1-3 sequences of SEQ ID NOs.: 55, 56, and 57, and the VL comprises the LCDR1-3 sequences of SEQ ID NOs.: 58, 59 and 60.

Such antigen binding protein may be or comprise an antibody, such as a full-length immunoglobulin or an antibody fragment, such as a Fab, a Fab′, a F(ab′)2, a scFv, a Fv fragment or a scFab.

The VL and VH may be joined by an amino acid linker, e.g., GGGGS (SEQ ID NO: 2), GGGGSGGGGS (SEQ ID NO: 3), GGGGSGGGGSGGGGS (SEQ ID NO: 4), GGGGSGGGGSGGGGSGGGGS (SEQ ID NO: 5), GGGGGSGGGGSGGGGSGGGGS (SEQ ID NO: 6), or GGGGSGGGGSGGGGSGGGGAS (SEQ ID NO: 7).

In certain embodiments, the antigen binding protein of the disclosure is or comprises a scFv with an amino acid sequence that is at least about 79%, 80%, 85%, 90%, 95%, 96%, 97%, 98%, 99% or 100% identical to the amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102 or SEQ ID NO: 103.

In certain embodiments, the antigen binding protein of disclosure is chemically or biologically modified, such as being glycosylated, PEGylated, PASylated, XTENylated or HESylated.

In certain embodiments, the antigen binding protein of the disclosure comprises a light chain and/or a heavy chain comprising an N-terminal and/or C-terminal truncation of 1, 2, 3, 4 or 5 amino acids. For example, in certain embodiments, the light chain comprises an N-terminal truncation of 1 or 2 amino acids, such as a terminal G. Additionally or alternatively, the glutamine (Q) or glutamate (E) at position 1 of the light chain and/or heavy chain of antigen binding protein of disclosure may be replaced by pyroglutamate (pE), for example, the light chain may comprise a pyroglutamate (pE) at position 1 of the instead of glutamine (Q).

In certain embodiments, the antigen binding protein of the disclosure is linked to or combined with a functional entity such as a detectable label, a therapeutic agent or a PK modifying moiety.

In one aspect, a chimeric antigen receptor (CAR) is provided, comprising the antigen binding protein of the disclosure. Accordingly, also provided is an immune cell expressing said CAR, in particular wherein the immune cell is a T cell.

In another aspect, an antibody drug conjugate (ADC) is provided, comprising the antigen binding protein of the disclosure.

In another aspect, a multispecific antigen binding protein is provided, comprising the antigen binding protein of the disclosure. Such multispecific antigen binding protein may e.g., be bispecific or trispecific. In certain embodiments thereof, the multispecific antigen binding protein comprises:
(1) a first antigen binding domain targeting an MHC-displayed SLLQHLIGL (SEQ ID NO: 139), i.e., the antigen binding domain of the disclosure; and
(2) an immune cell binding domain, such as a domain targeting CD3, TCRα, TCRβ, the α/β T Cell Receptor, CD16a, NKG2D, CD94/NKG2C, NKp30, NKp46, CD89, CD64, and CD32a on the surface of an immune cell, in particular a human immune cell, in particular CD3. A CD3 targeting domain may e.g. be derived from huSP34, huOKT3 or huUCHT1. In certain embodiments, the CD3 targeting domain is or comprises a Fab fragment, comprising:
(1) a heavy chain (HC) comprising a CH1 domain and the VH; and
(2) a light chain (LC) comprising a CL domain and the VL.

In certain embodiments thereof, such CD3 binding Fab domain comprises: an HC comprising an amino acid sequence that is at least about 90%, 95%, 96%, 97%, 98%, 99% or 100% identical to the amino acid sequence of SEQ ID NO: 104, and the VL domain comprises an amino acid sequence that is at least about 90%, 95%, 96%, 97%, 98%, 99% or 100% identical to the amino acid sequence of SEQ ID NO: 105, in particular having the CDR sequences of SEQ ID NOs 140-145.

The multispecific antigen binding protein may thus be bispecific and monovalent for each target and have or comprise a (scFv)2, BiTE, BIKE, Dart, diabody, Fab2, or a Fab-scFv scaffold. Thus, in certain embodiments, the multispecific antigen binding protein is a Fab-scFv, comprising or consisting of: (a) the scFv of SEQ ID NO: 93, being linked to the C-terminus of the HC of SEQ ID NO: 104; and a LC of SEQ ID NO: 105; (b) the scFv of SEQ ID NO: 94, being linked to the C-terminus of the HC of SEQ ID NO: 104; and a LC of SEQ ID NO: 105; (c) (i) the scFv of SEQ ID NO: 95, being linked to the C-terminus of the HC of SEQ ID NO: 104; and a LC of SEQ ID NO: 105; (d) the scFv of SEQ ID NO: 96, being linked to the C-terminus of the HC of SEQ ID NO: 104; and a LC of SEQ ID NO: 105; (e) the scFv of SEQ ID NO: 97, being linked to the C-terminus of the HC of SEQ ID NO: 104; and a LC of SEQ ID NO: 105; (f) the scFv of SEQ ID NO: 98, being linked to the C-terminus of the HC of SEQ ID NO: 104; and a LC of SEQ ID NO: 105; (g) the scFv of SEQ ID NO: 99, being linked to the C-terminus of the HC of SEQ ID NO: 104; and a LC of SEQ ID NO: 105; (h) the scFv of SEQ ID NO: 100, being linked to the C-terminus of the HC of SEQ ID NO: 104; and a LC of SEQ ID NO: 105; (i) the scFv of SEQ ID NO: 101, being linked to the C-terminus of the HC of SEQ ID NO: 104; and a LC of SEQ ID NO: 105; (j) the scFv of SEQ ID NO: 102, being linked to the C-terminus of the HC of SEQ ID NO: 104; and a LC of SEQ ID NO: 105; or (k) the scFv of SEQ ID NO: 103, being linked to the C-terminus of the HC of SEQ ID NO: 104; and a LC of SEQ ID NO: 105; or variants of said sequences that are at least about 90%, 95%, 96%, 97%, 98%, 99% or 100% identical to the amino acid sequences. In preferred embodiments, the CDRs of said variants remain unchanged compared to the parental sequence.

In certain embodiments, the multispecific antigen binding protein of the disclosure comprises or consists of: (a) the HC of SEQ ID NO: 106; and the LC of SEQ ID NO: 105; (b) the HC of SEQ ID NO: 107; and the LC of SEQ ID NO: 105; (c) the HC of SEQ ID NO: 108; and the LC of SEQ ID NO: 105; (d) the HC of SEQ ID NO: 109; and the LC of SEQ ID NO: 105; (e) the HC of SEQ ID NO: 110; and the LC of SEQ ID NO: 105; (f) the HC of SEQ ID NO: 111; and the LC of SEQ ID NO: 105; (g) the HC of SEQ ID NO: 112; and the LC of SEQ ID NO: 105; (h) the HC of SEQ ID NO: 113; and the LC of SEQ ID NO: 105; (i) the HC of SEQ ID NO: 114; and the LC of SEQ ID NO: 105; (j) the HC of SEQ ID NO: 115; and the LC of SEQ ID NO: 105; or (k) the HC of SEQ ID NO: 116; and the LC of SEQ ID NO: 105.

The multispecific antigen binding protein of the disclosure may further comprise a second antigen binding domain, such as one specifically binding to MHC-displayed SLLQHLIGL (SEQ ID NO: 139), in particular the antigen binding protein of the disclosure. In certain embodiments, the first and the second antigen binding domain are identical. Accordingly, in certain embodiments, the multispecific antigen binding protein is bispecific and bivalent for the pMHC target. Exemplary embodiments thereof include a (scFv)₃, tribody, Fab₂, Fab₃, Fab₄, or scFv-Fab-scFv (Fab-(scFvs)₂).

In one aspect, a multispecific antigen binding protein is provided having a Fab-(scFv)₂ scaffold, comprising
(a) a CD3 targeting Fab domain comprising the HCDRs of SEQ ID NOs: 140, 141 and 142 and/or the LCDRs of SEQ ID NO.: 143, 144 and 145; and
(b) two antigen binding scFvs specifically binding to MHC-displayed SLLQHLIGL (SEQ ID NO: 139), each scFv comprising:
(i) an antibody heavy chain variable (VH) domain comprising the HCDR amino acid sequences of SEQ ID NOs: 25, 26, and 27; and
(ii) an antibody light chain variable (VL) domain comprising the LCDR amino acid sequences of SEQ ID NOs.: 28, 29, and 30.

Specific sets of CDR variations are outlined in SEQ ID NOs.: 31-36, 37-42, 43-48, 49-54 and 55-60, respectively.

In certain embodiments, one pMHC binding scFv is linked to the C-terminus of the Fab domain heavy chain and the second pMHC binding scFv is linked to the C-terminus of the Fab domain light chain. In certain embodiments, the scFv comprises or consists of:
(1) (i) an antibody heavy chain variable (VH) domain that is at least about 90%, 95%, 96%, 97%, 98%, 99% or 100% identical to SEQ ID NO: 71; and (ii) an antibody light chain variable (VL) domain that is at least about 90%, 95%, 96%, 97%, 98%, 99% or 100% identical to SEQ ID NO: 72;
(2) (i) an antibody heavy chain variable (VH) domain that is at least about 90%, 95%, 96%, 97%, 98%, 99% or 100% identical to SEQ ID NO: 73; and (ii) an antibody light chain variable (VL) domain that is at least about 90%, 95%, 96%, 97%, 98%, 99% or 100% identical to SEQ ID NO: 74;
(3) (i) an antibody heavy chain variable (VH) domain that is at least about 90%, 95%, 96%, 97%, 98%, 99% or 100% identical to SEQ ID NO: 75; and (ii) an antibody light chain variable (VL) domain that is at least about 90%, 95%, 96%, 97%, 98%, 99% or 100% identical to SEQ ID NO: 76;
(4) (i) an antibody heavy chain variable (VH) domain that is at least about 90%, 95%, 96%, 97%, 98%, 99% or 100% identical to SEQ ID NO: 77; and (ii) an antibody light chain variable (VL) domain that is at least about 90%, 95%, 96%, 97%, 98%, 99% or 100% identical to SEQ ID NO: 78;
(5) (i) an antibody heavy chain variable (VH) domain that is at least about 90%, 95%, 96%, 97%, 98%, 99% or 100% identical to SEQ ID NO: 79; and (ii) an antibody light chain variable (VL) domain that is at least about 90%, 95%, 96%, 97%, 98%, 99% or 100% identical to SEQ ID NO: 80;
(6) (i) an antibody heavy chain variable (VH) domain that is at least about 90%, 95%, 96%, 97%, 98%, 99% or 100% identical to SEQ ID NO: 81; and (ii) an antibody light chain variable (VL) domain that is at least about 90%, 95%, 96%, 97%, 98%, 99% or 100% identical to SEQ ID NO: 82;
(7) (i) an antibody heavy chain variable (VH) domain that is at least about 90%, 95%, 96%, 97%, 98%, 99% or 100% identical to SEQ ID NO: 83; and (ii) an antibody light chain variable (VL) domain that is at least about 90%, 95%, 96%, 97%, 98%, 99% or 100% identical to SEQ ID NO: 84;
(8) (i) an antibody heavy chain variable (VH) domain that is at least about 90%, 95%, 96%, 97%, 98%, 99% or 100% identical to SEQ ID NO: 85; and (ii) an antibody light chain variable (VL) domain that is at least about 90%, 95%, 96%, 97%, 98%, 99% or 100% identical to SEQ ID NO: 86;
(9) (i) an antibody heavy chain variable (VH) domain that is at least about 90%, 95%, 96%, 97%, 98%, 99% or 100% identical to SEQ ID NO: 87; and (ii) an antibody light chain variable (VL) domain that is at least about 90%, 95%, 96%, 97%, 98%, 99% or 100% identical to SEQ ID NO: 88;
(10) (i) an antibody heavy chain variable (VH) domain that is at least about 90%, 95%, 96%, 97%, 98%, 99% or 100% identical to SEQ ID NO: 89; and (ii) an antibody light chain variable (VL) domain that is at least about 90%, 95%, 96%, 97%, 98%, 99% or 100% identical to SEQ ID NO: 90; or
(11) (i) an antibody heavy chain variable (VH) domain that is at least about 90%, 95%, 96%, 97%, 98%, 99% or 100% identical to SEQ ID NO: 91; and (ii) an antibody light chain variable (VL) domain that is at least about 90%, 95%, 96%, 97%, 98%, 99% or 100% identical to SEQ ID NO: 92.

In preferred embodiments, the CDRs of the recited variants remain unchanged compared to the parental sequence.

In specific embodiments, the multispecific antigen binding protein described herein is a Fab-(scFv)₂, comprising or consisting of
(1) (i) a heavy chain (HC) that is at least about 90%, 95%, 96%, 97%, 98%, 99% or 100% identical to SEQ ID NO: 117; and (ii) a light chain (LC) that is at least about 90%, 95%, 96%, 97%, 98%, 99% or 100% identical to SEQ ID NO: 118;
(2) (i) a heavy chain (HC) that is at least about 90%, 95%, 96%, 97%, 98%, 99% or 100% identical to SEQ ID NO: 119; and (ii) a light chain (LC) that is at least about 90%, 95%, 96%, 97%, 98%, 99% or 100% identical to SEQ ID NO: 120;
(3) (i) a heavy chain (HC) that is at least about 90%, 95%, 96%, 97%, 98%, 99% or 100% identical to SEQ ID NO: 121; and (ii) a light chain (LC) that is at least about 90%, 95%, 96%, 97%, 98%, 99% or 100% identical to SEQ ID NO: 122;
(4) (i) a heavy chain (HC) that is at least about 90%, 95%, 96%, 97%, 98%, 99% or 100% identical to SEQ ID NO: 123; and (ii) a light chain (LC) that is at least about 90%, 95%, 96%, 97%, 98%, 99% or 100% identical to SEQ ID NO: 124;
(5) (i) a heavy chain (HC) that is at least about 90%, 95%, 96%, 97%, 98%, 99% or 100% identical to SEQ ID NO: 125; and (ii) a light chain (LC) that is at least about 90%, 95%, 96%, 97%, 98%, 99% or 100% identical to SEQ ID NO: 126;
(6) (i) a heavy chain (HC) that is at least about 90%, 95%, 96%, 97%, 98%, 99% or 100% identical to SEQ ID NO: 127; and (ii) a light chain (LC) that is at least about 90%, 95%, 96%, 97%, 98%, 99% or 100% identical to SEQ ID NO: 128;
(7) (i) a heavy chain (HC) that is at least about 90%, 95%, 96%, 97%, 98%, 99% or 100% identical to SEQ ID NO: 129; and (ii) a light chain (LC) that is at least about 90%, 95%, 96%, 97%, 98%, 99% or 100% identical to SEQ ID NO: 130;
(8) (i) a heavy chain (HC) that is at least about 90%, 95%, 96%, 97%, 98%, 99% or 100% identical to SEQ ID NO: 131; and (ii) a light chain (LC) that is at least about 90%, 95%, 96%, 97%, 98%, 99% or 100% identical to SEQ ID NO: 132;
(9) (i) a heavy chain (HC) that is at least about 90%, 95%, 96%, 97%, 98%, 99% or 100% identical to SEQ ID NO: 133; and (ii) a light chain (LC) that is at least about 90%, 95%, 96%, 97%, 98%, 99% or 100% identical to SEQ ID NO: 134;
(10) (i) a heavy chain (HC) that is at least about 90%, 95%, 96%, 97%, 98%, 99% or 100% identical to SEQ ID NO: 135; and (ii) a light chain (LC) that is at least about 90%, 95%, 96%, 97%, 98%, 99% or 100% identical to SEQ ID NO: 136; or
(11) (i) a heavy chain (HC) that is at least about 90%, 95%, 96%, 97%, 98%, 99% or 100% identical to SEQ ID NO: 137; and (ii) a light chain (LC) that is at least about 90%, 95%, 96%, 97%, 98%, 99% or 100% identical to SEQ ID NO: 138.

Such multispecific antigen binding protein may comprise an N-terminal and/or C-terminal truncation of 1, 2, 3, 4, or 5 amino acids in the light chain and/or heavy chain. For example, the light chain may comprise an N-terminal truncation of 1 or 2 amino acids. Additionally or alternatively, the multispecific antigen binding protein of disclosure may comprise a pyroglutamate (pE) at position 1 instead of glutamine (Q) or glutamate (E) of the light chain and/or heavy chain. For example, the light chain may comprise a pyroglutamate (pE) at position 1 instead of glutamine (Q) or glutamate (E) of the light chain.

In another aspect, a chimeric antigen receptor (CAR) is provided, comprising an antigen binding protein disclosed herein as well as an immune cell expressing such CAR.

In another aspect, an antibody-drug-conjugate (ADC) is provided, comprising an antigen binding protein disclosed herein.

In another aspect, an isolated polynucleotide encoding an antigen binding protein disclosed herein, antibody-drug-conjugate (ADC), or a CAR according to the present disclosure is provided.

In another aspect, an isolated polynucleotide is provided, encoding an antigen binding protein of the disclosure.

In another aspect, a vector comprising the isolated polynucleotide is provided.

In another aspect, a host cell comprising the isolated polynucleotide and/or vector according to the present disclosure herein is provided.

In another aspect, a method of producing an antigen binding protein, a CAR or an ADC according to the present disclosure is provided.

In another aspect, a pharmaceutical composition comprising an antigen binding protein as described herein, a polynucleotide as described herein, a vector as described herein, an ADC as described herein, a CAR described herein, or a cell as described herein is provided, further including a pharmaceutically acceptable carrier.

The antigen binding protein, the CAR, the ADC and particularly the pharmaceutical composition according to the present disclosure can be used as medicament and in the treatment of a disease, in particular ovarian, NSCLC, melanoma, synovial sarcoma cancer.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 depicts killing of HLA-A*02:01-positive PRAME-positive OV56 and NCI-H1755 cancer cells and HLA-A*02:01-positive PRAME-negative TYK-nu and Colo678 cancer cells mediated by HLA-A*02:01/PRAME specific rabbit hit M2638 in a Fab-scFv format and its bivalent counterpart M2873 in a Fab-(scFv)₂ format.

FIG. 2 depicts safety of M2638 in HLA-A*02:01-positive human primary lung fibroblasts and cardiac endothelial cells. HLA-A*02:01-positive PRAME-positive OV56 cancer cell line served as a positive control and PBMCs only with the compound as a negative control.

FIG. 3A-FIG. 3B depict potency of humanized and optimized bispecific molecules on cancer cell lines. FIG. 3A: Killing of HLA-A*02:01-positive PRAME-positive NCI-H1703, NCI-H1755, and/or OV56 cancer cells and HLA-A*02:01-positive PRAME-negative TYK-nu, DBTRG-05MG, SK-MEL-30 and/or Colo678 cancer cells mediated by the humanized and optimized bispecific compounds M2913, M3076, M3182, M3202, M3283, M3284, M3293 in a Fab-scFv format, and M3134, M3167, M3275, M3279, M3288, M3289, M3290, M3291, M3292 and M3326 in a Fab-(scFv)₂ format.

FIG. 3B: Granzyme B release of HLA-A*02:01-positive PRAME-positive OV56, NCI-H1703, NCI-H1755, COV813 and/or SK-MEL-5 cancer cells and HLA-A*02:01-positive PRAME-negative SW620, Colo678, SK-MEL-30 and/or CFPAC-1 cancer cells mediated by the humanized and optimized bispecific compound M3167 in a Fab-(scFv)₂ format.

FIG. 4A-FIG. 4B depict potency of M3167 compared to Comparator 1 and Comparator 2 on cancer cell lines. FIG. 4A: Killing of HLA-A*02:01-positive PRAME-positive OV56 cancer cells and HLA-A*02:01-positive PRAME-negative Colo678 cancer cells mediated by the humanized and optimized M3167 in a Fab-(scFv)₂ format, Comparator 1 or Comparator 2. FIG. 4B: Granzyme B and Interferon 7 release of HLA-A*02:01-positive PRAME-positive OV56 cancer cells mediated by the humanized and optimized M3167 in a Fab-(scFv)₂ format, Comparator 1 or Comparator 2.

FIG. 5A-FIG. 5C depict safety of HLA-A*02:01/PRAME specific humanized and optimized bispecific compounds tested for their reactivity on human primary cells. FIG. 5A: Compounds M2913, M3076, M3182, M3202 in a Fab-scFv format, and M3167 and M3275 in a Fab-(scFv)₂ format were tested in HLA-A*02:01-positive human primary aortic smooth muscle cells (HAoSMC_735), tracheal smooth muscle cells (HTSMC_084), cardiac fibroblasts (HCF_251), pulmonary fibroblasts (HPF_197) and/or skeletal muscle cells (SkMC_683). HLA-A*02:01-positive PRAME-positive OV56 cancer cell line served as a positive control. FIG. 5B: Compound M3167 was additionally tested on HLA-A*02:01-positive human primary cardiomyocytes (HCM_718, HCM_693, HCM_746), hepatocytes (Human Hepatocytes_11621), astrocytes (NHA_72445), cardiac microvascular endothelial cells (HCMEC_693, HCMEC_798), cardiac fibroblasts (HCF_243), pulmonary microvascular endothelial cells (HPMEC_760, HPMEC_197), aortic endothelial cells (HAEC_22171), small airway epithelial cells (SAEC_33436), pulmonary artery smooth muscle cells (PASMC_01036), skeletal muscle cells (SkMC_683), aortic smooth muscle cells (HAoSMC_735) an/or PBMCs from four different donors. HLA-A*02:01-positive PRAME-positive OV56 cancer cell line served as a positive control. FIG. 5C: Safety of compound M3167, Comparator 1 and Comparator 2 was tested on HLA-A*02:01-positive human primary renal proximal tubule epithelial cells (RPTEC_82573, RPTEC_49985, RPTEC_95730) and/or renal cortical epithelial cells (HRCE_49986). HLA-A*02:01-positive PRAME-positive OV56 cancer cell line served as a positive control.

FIG. 6A-FIG. 6B depict exemplary multispecific antigen binding protein formats. FIG. 6A: a Fab-(scFv)₂ scaffold, an exemplary embodiment of a bispecific antigen binding protein as described herein, being bivalent for pMHC binding and monovalent for T cell binding. The Fab is directed against a T cell antigen, e.g., CD3. A first pMHC binding scFv is attached via a linker to the C-terminal of the Fab heavy chain (CH1) and a second pMHC binding scFv is attached via a linker to the C-terminal of the Fab light chain (CL domain). FIG. 6B: a Fab-scFv scaffold, an exemplary embodiment of a bispecific antigen binding protein being monovalent for pMHC binding and monovalent for T cell binding. Here, a single pMHC binding scFv is attached via a linker to the C-terminal of the Fab heavy chain.

FIG. 7 depicts stability measurements of M3167 and M3292 in a Fab-(scFv)₂ format compared to the compounds Comparator 1 and Comparator 2. Stability was assessed via SEC quantification of the compound monomer after incubation for seven days at 34° C. at high concentrations in artificial subcutaneous fluid buffer (pH 7.4).

FIG. 8 depicts copy numbers of HLA-A*02:01/PRAME complexes per cell on lung adenocarcinoma biopsies determined by mass spectrometry.

FIG. 9 depicts differential PRAME RNA expression levels in various malignancies compared to the corresponding healthy tissues by using the web-based tool GEPIA (Gene Expression Profiling Interactive Analysis).

### DETAILED DESCRIPTION

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of the term “including,” as well as other forms, such as “includes” and “included,” is not limiting.

So that the invention may be more readily understood, certain terms are first defined.

### Antigen Binding Proteins

As used herein, the term “PRAME” (Preferentially Expressed Antigen in Melanoma) refers to a human protein belonging to the cancer/testis antigen (CTA) protein family (for sequence information, see Uniprot Acc. No. P78395). CTAs are a class of cancer antigens that are mainly expressed in tumors and testicular tissues but display low to no expression in other normal tissues. PRAME is also referred to as MAPE (melanoma antigen preferentially expressed in tumors) and OIP-4 (OPA-interacting protein 4). The antigen binding proteins of the disclosure bind the MHC-displayed SLLQHLIGL (SEQ ID NO: 139) peptide, said peptide being derived from the PRAME protein and corresponds to amino acids 425-433 of the full-length protein.

“CD3”, the cluster of differentiation 3 co-receptor (or co-receptor complex) of the T cell receptor, is a complex composed of four distinct chains. In mammals, the complex contains a CD3γ (gamma) chain/subunit, a CD3δ (delta) chain/subunit, and two CD3ε (epsilon) chains/subunits. Reference to CD3 as the cell surface protein of an immune cell is made herein throughout. The term “CD3” refers to any native CD3 from any vertebrate source, including primates. In certain embodiments, the antigen binding proteins of the disclosure specifically bind to human CD3, in particular to the CD3ε (epsilon) chain/subunit of CD3 (see e.g., UniProt (www.uniprot.org) accession no. P07766 (version 189), or NCBI (www.ncbi.nlm.nih.gov/) RefSeq NP_000724.1.) The CD3 molecule may be the full-length, unprocessed CD3 molecule or a fragment or variant thereof that e.g., results from processing in the cell. For example, such variants may be naturally occurring variants like splice variants or allelic variants. In certain embodiments, the antigen binding proteins disclosed herein bind to an epitope of CD3 that is conserved among the CD3 antigens from different species, such as non-human primates (e.g., cynomolgus monkeys) or rodents (e.g., mice, rats). In certain embodiments, the antigen binding proteins are not cross-reactive with a CD3 antigen from rodents (e.g., mouse or rat) or minipigs.

As used herein, the term “antigen binding protein” refers to a protein that specifically binds to or is immunologically reactive with an antigen or epitope. The term “antigen binding protein” includes “antibody” and more particularly “antibody fragment”, but also non-immunoglobulin-based binding proteins as described in further detail in Simeon et al. (Protein Cell. 2018. 9(1): 3-14) and Olaleye et al. (Biomolecules. 2021. 11(12): 1791), each of which is incorporated herein by reference. Non-limiting examples of non-immunoglobulin-based binding proteins include DARPins, affimers, monobodies, anticalins, fynomers, and affibodies.

The term “antibody” refers to an immunoglobulin molecule or immunoglobulin derived molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)2 fragments, Fv fragments, recombinant IgG (rIgG) fragments, single chain variable fragments (scFv) and single domain antibodies (e.g., sdAb, sdFv, nanobody, VHH fragments). An “antibody” as used herein may thus be a single domain antibody or comprise at least one variable light and at least one variable heavy chain. In one embodiment, the at least one variable light and at least one variable heavy chain are displayed as a single polypeptide chain. In certain embodiments, the term “antibody” or “antigen binding protein” includes germline derived antibodies. The term “antibody” or “antigen binding protein” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments of an immunoglobulin molecule.

In certain embodiments, the antigen binding protein is not a T cell receptor (TCR), including but not limited to, a soluble TCR.

In certain embodiments, the antigen binding protein is multispecific, i.e., binds to two or more different target molecules or to two or more epitopes on the same target molecule. In certain embodiments, the antigen binding protein is bispecific and e.g., binds to two different target molecules or to two epitopes on the same target molecule. In certain embodiments, the antigen binding protein is trispecific and e.g., binds to at least three different target molecules. In certain embodiments thereof, a trispecific antigen binding protein comprises three binding sites and binds to antigens on at least two distinct cells.

The antigen binding protein may be monovalent or multivalent, i.e., having one or more antigen binding sites. Non-limiting examples of monovalent antigen binding proteins include scFv, Fab, scFab, dAb, VHH, V(NAR), DARPins, affilins and nanobodies. A multivalent antigen binding protein can have two, three, four or more antigen binding sites. Non-limiting examples of multivalent antigen binding proteins include full-length immunoglobulins, F(ab′)₂ fragments, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv2-Fc, FynomABs, quadroma, CrossMab, DuoBody, triabodies and tetrabodies. In some embodiments, the multivalent antigen binding protein is bivalent, i.e., two binding sites are present. In some embodiments, the multivalent antigen binding protein is bispecific, i.e., the antigen binding protein is directed against two different targets or two different target sites on one target molecule. In some embodiments, the multivalent antigen binding protein includes more than two, e.g., three or four different binding sites for three or four, respectively, different antigens. Such antigen binding protein is multivalent and multispecific, in particular tri- or tetra-specific, respectively.

In some embodiments, the antigen binding proteins are multispecific (e.g., bispecific), such as, without being limited to, diabodies, single-chain diabodies, DARTs, BiTEs, BIKEs, tandem scFvs or IgG-like asymmetric heterobispecific antibodies. In certain embodiments, one or the binding specificities of the multispecific antigen binding protein is an immune cell engager (i.e., comprising affinity to a cell surface protein of an immune cell, also referred to as “immune cell binding domain”). Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells includes, without being limited to, CD3, TCRα, TCRβ, CD16, NKG2D, CD94/NKG2C, NKp30, NKp46, CD89, CD64, and CD32. In certain embodiments, the immune cell target antigen is CD3. In certain embodiments, the multispecific antigen binding protein is a bispecific targeting CD3 and MHC-displayed SLLQHLIGL (SEQ ID NO: 139). In certain embodiments, said bispecific antigen binding protein is monovalent for CD3 and bivalent for MHC-displayed SLLQHLIGL (SEQ ID NO: 139).

As used herein, the term “monoclonal antibody” refers to an antibody obtained from a population of substantially homogeneous antibodies, i.e., antibodies that bind to the same epitope and/or are identical in sequence. A population of polyclonal antibodies, in contrast, will bind to multiple epitopes and comprises antibodies of different sequences. A monoclonal antibody preparation may or may not comprise to a minor extent variant antibodies, e.g., due to naturally occurring mutations. Such variants may e.g., be generated through posttranslational modifications, such as clipping at the N-terminal or C-terminal end of the light and/or heavy chain, or pyroglutamate formation on the N terminus of the polypeptide chain (see e.g., Liu Y D, et al. J Biol Chem. 2011 Apr. 1; 286(13):11211-7). Depending on the methods and antibody used, the percentage of variants in the mixture varies, and may involve substantially all antibodies produced, or a very low percentage.

As used herein, a “single-chain variable fragment” (scFv) is an antigen binding protein comprising a heavy chain variable domain (VH) linked to a light chain variable domain (VL). The VH and VL domains of the scFv are linked via any appropriate art recognized linker. Such linkers include, but are not limited to, repeated GGGGS (SEQ ID NO: 2) amino acid sequences or variants thereof. The scFv is generally free of antibody constant domain regions, although an scFv of the disclosure may be linked or attached to antibody constant domain regions (e.g., antibody Fc domain) to alter various properties of the scFv, including, but not limited to, increased serum or tissue half-life. An scFv generally has a molecular weight of about 25 kDa and a hydrodynamic radius of about 2.5 nm. As used herein, a “Fab fragment” or “Fab” or “Fab domain” is an antibody fragment comprising a light chain fragment comprising a variable light (VL) domain and a constant domain of the light chain (CL), and variable heavy (VH) domain and a first constant domain (CH1) of the heavy chain. A F(ab′)₂ comprises two antigen-binding regions joined at the hinge through disulfides.

As used herein, a “VHH”, “nanobody”, “heavy-chain only antibody”, “single domain antibody”, or “sdAb” is an antigen binding protein comprising a single heavy chain variable domain derived from the species of the Camelidae family, which includes camels, llama, alpaca. A VHH generally has a molecular weight of about 15 kDa.

The antigen binding proteins of the disclosure may comprise one or more linkers for linking the domains of the antigen binding protein (e.g., linking a VH and VL to form a scFv, or linking multiple binding domains to form a multispecific antigen binding protein). Illustrative examples of linkers include glycine polymers (Gly)ₙ; glycine-serine polymers (GlyₙSer)ₙ, where n is an integer of at least one, two, three, four, five, six, seven, or eight; glycine-alanine polymers; alanine-serine polymers; and other flexible linkers known in the art.

Glycine and glycine-serine polymers are relatively unstructured, and therefore may be able to serve as a neutral tether between domains of fusion proteins such as the antigen binding proteins described herein. Glycine accesses significantly more phi-psi space than other small side chain amino acids and is much less restricted than residues with longer side chains (Scheraga, Rev. Computational Chem. 1: 1173-142 (1992)). A person skilled in the art will recognize that in particular embodiments, certain antigen binding protein formats can include linkers that are all or partially flexible, such that the linker can include flexible linker stretches as well as one or more stretches that confer less flexibility to provide a desired structure.

Linker sequences can however be chosen to resemble natural linker sequences, for example, using the amino acid stretches corresponding to the beginning of human CH1 and Cκ sequences or amino acid stretches corresponding to the lower portion of the hinge region of human IgG.

The peptide linkers connecting VL and VH domains in the scFv moieties are typically flexible linkers generally composed of small, non-polar or polar residues such as, e.g., Gly, Ser and Thr. A particularly exemplary linker connecting the variable domains of the scFv moieties is the (Gly₄Ser)₄ linker (SEQ ID NO: 4), where 4 is the exemplary number of repeats of the motif.

Linkers connecting the scFv antigen binding proteins to the Fab domain are also envisioned. In certain embodiments, the scFv antigen binding proteins are linked to the CH1 and CL domains of the Fab with a Gly-Ser linker. In certain embodiments, the linker comprises the amino acid sequence GGGGS (SEQ ID NO: 2). In certain embodiments, the linker comprises 3-5 repetitions of the GGGGS (SEQ ID NO: 2) motif. In certain embodiments, the amino acid linker comprises the amino acid sequence GGGGSGGGGSGGGGS (SEQ ID NO: 4), GGGGSGGGGSGGGGSGGGGS (SEQ ID NO: 5), or GGGGSGGGGSGGGGSGGGGAS (SEQ ID NO: 6). Alternatively, linker sequences connecting the variable domains may include glycine polymers (G)ₙ; glycine-alanine polymers; alanine-serine polymers; and other flexible linkers known in the art.

Other exemplary linkers include, but are not limited to the following amino acid sequences: GGG; DGGGS (SEQ ID NO: 22); TGEKP (SEQ ID NO: 8) (Liu et al, Proc. Natl. Acad. Sci. 94: 5525-5530 (1997)); GGRR (SEQ ID NO: 9); (GGGGS)ₙ (SEQ ID NO: 1) wherein n=1, 2, 3, 4 or 5 (Kim et al, Proc. Natl. Acad. Sci. 93: 1156-1160 (1996)); EGKSSGSGSESKVD (SEQ ID NO:10) (Chaudhary et al., Proc. Natl. Acad. Sci. 87: 1066-1070 (1990)); KESGSVSSEQLAQFRSLD (SEQ ID NO: 11) (Bird et al., Science 242:423-426 (1988)), GGRRGGGS (SEQ ID NO: 12); LRQRDGERP (SEQ ID NO: 13); LRQKDGGGSERP (SEQ ID NO: 14); and GSTSGSGKPGSGEGSTKG (SEQ ID NO: 15) (Cooper et al, Blood, 101(4): 1637-1644 (2003)). Alternatively, flexible linkers can be rationally designed using a computer program capable of modeling the 3D structure of proteins and peptides or by phage display methods.

The antibodies may comprise a variable light (VL) domain and a variable heavy (VH) domain. Each VL and VH domain further comprises a set of three CDRs.

As used herein, the term “complementarity determining region” or “CDR” refers to non-contiguous sequences of amino acids within antibody variable regions, which confer antigen specificity and affinity. In general, there are three CDRs in each heavy chain variable domain (HCDR1, HCDR2, HCDR3) and three CDRs in each light chain variable domain (LCDR1, LCDR2, LCDR3). “Framework regions” or “FRs” are known in the art to refer to the non-CDR portions of the variable domains of the heavy and light chains. In general, there are four FRs in each heavy chain variable domain (HFR1, HFR2, HFR3, and HFR4), and four FRs in each light chain variable domain (LFR1, LFR2, LFR3, and LFR4). Accordingly, an antibody variable region amino acid sequence can be represented by the formula FR1-CDR1-FR2-CDR2-FR3-CDR3-FR4. Each segment of the formula, i.e., FR1, CDR1, FR2, CDR2, FR3, CDR3, and FR4, represents a discrete amino acid sequence (or a polynucleotide sequence encoding the same) that can be mutated, including one or more amino acid substitutions, deletions, and insertions. In certain embodiments, an antibody variable light chain amino acid sequence can be represented by the formula LFR1-LCDR1-LFR2-LCDR2-LFR3-LCDR3-LFR4. In certain embodiments, an antibody variable heavy chain amino acid sequence can be represented by the formula HFR1-HCDR1-HFR2-HCDR2-HFR3-HCDR3-HFR4.

The precise amino acid sequence boundaries of a given CDR or FR can be readily determined using any of a number of well-known schemes, including those described by Kabat et al. (1991), “Sequences of Proteins of Immunological Interest,” 5^{th} Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1, below, lists exemplary position boundaries of LCDR1, LCDR2, LCDR3 and HCDR1, HCDR2, HCDR3 of an antibody, as identified by Kabat, Chothia, and Contact schemes, respectively. For CDRH1, residue numbering is listed using both the Kabat and Chothia numbering schemes. CDRs are located between FRs, for example, with CDRL1 located between LFR1 and LFR2, and so forth. It is noted that because the shown Kabat numbering scheme places insertions at H35A and H35B, the end of the Chothia CDRH1 loop when numbered using the shown Kabat numbering convention varies between H32 and H34, depending on the length of the loop.

TABLE 1
Exemplary Position Boundaries of CDRs

| CDR | Kabat | Chothia | Contact |
|---|---|---|---|
| LCDR1 | L24-L34 | L24-L34 | L30-L36 |
| LCDR2 | L50-L56 | L50-L56 | L46-L55 |
| LCDR3 | L89-L97 | L89-L97 | L89-L96 |
| HCDR1 | H31-H35B | H26-H32 . . . 34 | H30-H35B |
| (Kabat Numbering¹) | | | |
| HCDR1 | H31-H35 | H26-H32 | H30-H35 |
| (Chothia Numbering²) | | | |
| HCDR2 | H50-H65 | H52-H56 | H47-H58 |
| HCDR3 | H95-H102 | H95-H102 | H93-H101 |

¹Kabat et al. (1991), “Sequences of Proteins of Immunological Interest,” 5^{th} Ed. Public Health Service, National Institutes of Health, Bethesda, MD
²Al-Lazikani et al. (1997), J. Mol. Biol. 273: 927-948

Thus, unless otherwise specified, a “CDR” or “complementary determining region,” or individual specified CDRs (e.g., CDRH1, CDRH2), of a given antibody or fragment thereof, such as a variable domain thereof, should be understood to encompass a (or the specific) complementary determining region as defined by Kabat. However, CDRH1 as used herein is defined by residues H30-H35B (Kabat numbering) or H30-H35 (Chothia numbering). Likewise, unless otherwise specified, an “FR” or “framework region,” or individual specified FRs (e.g., “HFR1,” “HFR2”) of a given antibody or fragment thereof, such as a variable domain thereof, should be understood to encompass a (or the specific) framework region as defined by Kabat. In some instances, the scheme for identification of a particular CDR or FR is specified, such as the CDR as defined by the Kabat, Chothia, or Contact method. In other cases, the particular amino acid sequence of a CDR or FR is given.

In certain embodiments, the antigen binding proteins disclosed here are rabbit antibodies or rabbit-derived antibodies. In certain embodiments, the rabbit antibodies are humanized. As used herein, the term “humanized” or “humanization” refers to an antibody that has been altered to make it more like a human antibody. Non-human antibodies, such as rabbit antibodies, would elicit a negative immune reaction if administered to a human for therapy. It is therefore advantageous to humanize rabbit antibodies for later therapeutic use. In certain embodiments, the antibodies are humanized through resurfacing (i.e., remodel the solvent-accessible residues of the non-human framework such that they become more human-like). Resurfacing strategies are described in more detail in WO2004/016740, WO2008/144757, and WO2005/016950, each of which is incorporated herein by reference. In certain embodiments, the antibodies are humanized through CDR grafting (i.e., inserting the CDRs of the rabbit antibody into a human antibody acceptor framework). Grafting strategies and human acceptor frameworks are described in more detail in WO2009/155726, incorporated herein by reference.

As used herein, “sequence identity” between two polypeptides is determined by comparing the amino acid sequence of one polypeptide to the sequence of a second polypeptide. Similarly, “sequence identity” between two polynucleotides is determined by comparing the nucleotide sequence of one polynucleotide to the sequence of a second polynucleotide. The terms “% identical”, “% identity” or similar terms are intended to refer, in particular, to the percentage of nucleotides or amino acids (as applicable) which are identical after the sequences to be compared have been aligned to yield maximum identity, potentially introducing gaps. Said percentage may be purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. For example, the optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using the algorithms (GAP, BESTFIT, FASTA, BLAST P, BLAST N and TFASTA in Wisconsin Genetics Software Package, Genetics Computer Group, 575 Science Drive, Madison, Wis., or Clustal Omega). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). Typically, the % identity is determined over the entire length of the query sequence on which the analysis is performed.

A variant polypeptide, such as an antigen binding protein, may contain one or more substitutions (e.g., conservative substitutions), insertions, or deletions relative to the reference sequence. In certain embodiments, the variant polypeptide comprises one, two or three substitutions, insertions and/or deletions relative to the reference sequence. Substitutions, insertions, or deletions may result in the change of one or more biophysical parameters but particularly preferred are tolerated changes so that the polypeptide retains the desired activity. In some embodiments, the variant polypeptide comprises one or more tolerated substitutions, such as conservative substitutions. In certain embodiments, such variant polypeptide maintains physically, biologically, chemically and/or functionally the properties of the corresponding parental sequence.

“Specifically recognizes” or “specifically binds” refers to the ability of the antigen binding proteins to bind selectively to the antigen in contrast to non-specific interactions with unrelated proteins which do not comprise the binding epitope. Suitable assays for determining the specific binding are described below. In certain embodiments, the equilibrium dissociation constant (K_{D}) of an antigen binding protein to an unrelated protein is less than about 50-fold lower than the one of the antigen binding protein to its antigen, as e.g., determined by SPR.

As used herein, the term “affinity” (or “binding affinity” as used interchangeably herein) refers to the strength of the interaction between an antibody's antigen binding site and the epitope to which it binds. As readily understood by those skilled in the art, the affinity of an antigen binding protein or in particular an antibody may be reported as an equilibrium dissociation constant (K_{D}) in molarity (M). The equilibrium dissociation constant K_{D} is calculated from the association rate constant kₐ (having the unit M⁻¹s⁻¹) and the dissociation rate constant k_{d} (having the unit s⁻¹) by k_{d}/kₐ. The antibodies of the disclosure may have K_{D} values in the range of 10⁻⁸ to 10⁻¹⁴ M.

The ability of an antibody to bind to a specific antigenic determinant (e.g., a target peptide-MHC) can be measured either through an enzyme-linked immunosorbent assay (ELISA) or other techniques familiar to one of skill in the art, e.g., surface plasmon resonance (SPR) technique (conducted e.g. using a BIAcore instrument) (Liljeblad et al., Glyco J 17, 323-329 (2000)), and traditional binding assays (Heeley, Endocr Res 28, 217-229 (2002)). Generally, kinetic rate constants can be determined at temperatures in the range of 15° C. to 37° C. The present specification makes reference to kinetic rate constants determined by SPR throughout. Typically, in embodiments pertaining to each reference to SPR throughout the present specification, association rate constant values, dissociation rate constant values and equilibrium dissociation constant values recited herein are determined by SPR at 25° C. based on the monovalent antigen binding protein. Preferably, the SPR-based system used is a Biacore SPR system. The skilled person will appreciate that the binding parameters can be measured in the context of the monovalent or bivalent bi-, tri- or multispecific constructs. In certain embodiments, affinity values recited herein are determined in a monovalent Fab-scFv format, wherein the Fab targets CD3 and the scFv the pMHC.

In certain embodiments, the antigen binding protein is not a T cell receptor (TCR), including but not limited to, a soluble TCR. As used herein, the term “T cell receptor” or “TCR” refers to a heterodimeric protein comprised of two different chains (TCRα and TCRβ), which structurally belong to the immunoglobulin (Ig) superfamily. The extracellular portion of each chain is composed of variable (“Vα” and “Vβ”) and constant (“Cα” and “Cβ”) domains, and a hinge region, where the formation of a stabilizing disulfide bond occurs. The intracellular region forms a non-covalent interaction with another trans-membrane protein, CD3, which in the case of the correct target recognition leads to a series of conformational changes and a first T cell activation signal. Recognition and binding of peptide-MHC (pMHC) by a TCR is governed by the six hypervariable loops, termed complementarity determining regions (CDRs), located on the variable domains of the TCRα (CDRα1, CDRα2, CDRα3) and TCRβ (CDRβ1, CDRβ2, CDRβ3). CDR3 loops (CDRα3 and CDRβ3) lead the recognition of the processed antigen with the support of CDRα1 and CDRβ1, that have been implicated in the recognition of the N- and C-terminal amino acids of the presented peptide, respectively (Rudolph et al. Annu Rev Immunol. 24:419-66. 2006). Recognition of the MHC is typically achieved through the interaction with CDRα2 and CDRβ2. The high sequence diversity of the TCR is achieved through V(D)J recombination process, in which the variable domain is generated from a combination of genes: V (variable) and J (joining) for both TCRα and TCRβ, and an additional D (diversity) gene for TCRβ. The high antigen specificity of the TCR is controlled by the thymic maturation process, in which the self-reacting T cells are negatively selected. TCR affinity towards the specific pMHC and the functional avidity are the key factors controlling T-cell activation. A critical role in antigen recognition, however, is played by the affinity (K_{D}), i.e., the strength of binding between the TCR and the cell-displayed pMHC (Tian et al. J Immunol. 179:2952-2960. 2007). The physiological affinities of TCRs range from 1 μM to 100 μM (Davis et al. Annu Rev Immunol. 16:523-544. 1998), which, in comparison to antibodies, is relatively low.

As used herein, the term “peptide-MHC” or “pMHC” or “pMHC complex” as used interchangeably herein, refers to a major histocompatibility complex (MHC) molecule (MHC-I or -II) with an antigenic peptide bound in a peptide binding pocket of the MHC. As is known in the art, MHC molecules present peptides, in particular antigenic peptides, on the surface of cells to be recognized by immune cells. Accordingly, as will be appreciated by a skilled artisan, the term “pMHC” as used herein refers to a complex of an MHC molecule and a peptide, in particular an antigenic peptide, presented by the MHC molecule. This is commonly known as MHC-restricted antigen presentation. Accordingly, the peptide targeted by the pMHC binding domains is an MHC-restricted peptide. The peptide can thus be considered as target peptide or target antigenic peptide. Further, in accordance with the present disclosure, the terms “target pMHC binding domain” and “pMHC binding domain” may be used interchangeably herein, and in any case refer to the at least first and at least second pMHC binding domains referred to herein throughout. The terms “target peptide/antigen presented by an MHC molecule/complex” and “MHC restricted target peptide/antigen”, or similar expressions used throughout the present specification, may be used interchangeably herein.

While MHCs occur in all vertebrates, the MHC in humans is known as HLA (human leukocyte antigen). HLA is highly polygenic and can be broadly divided into three classes of MHC molecules, class I, class II and class III. Moreover, HLA genes have the highest level of polymorphism of the human genome. The target peptide may be presented on an MHC class I complex (such as of serotype HLA-A, HLA-B, HLA-C, HLA-E, HLA-F, HLA-G, HLA-K or HLA-L, or their respective subtypes) or an MHC class II complex (such as the serotypes HLA-DP, HLA-DQ, HLA-DR, DM or DO, or their respective subtypes). The HLA-A protein constitutes the alpha chain of the respective class I MHC (major histocompatibility complex) protein, which further comprises a beta 2 microglobulin subunit. The extracellular region of the alpha chain comprises three immunoglobulin-like domains (α1, α2, and α3). The α1 and α2 domains form a peptide-binding groove wherein the HLA-restricted peptide is typically located. Accordingly, peptides displayed by the HLA complex may be identified as pHLA herein. The terms “HLA-displayed”, “HLA-presented”, “loaded onto HLA”, “peptide-loaded HLA” and “HLA-restricted” are used interchangeably herein.

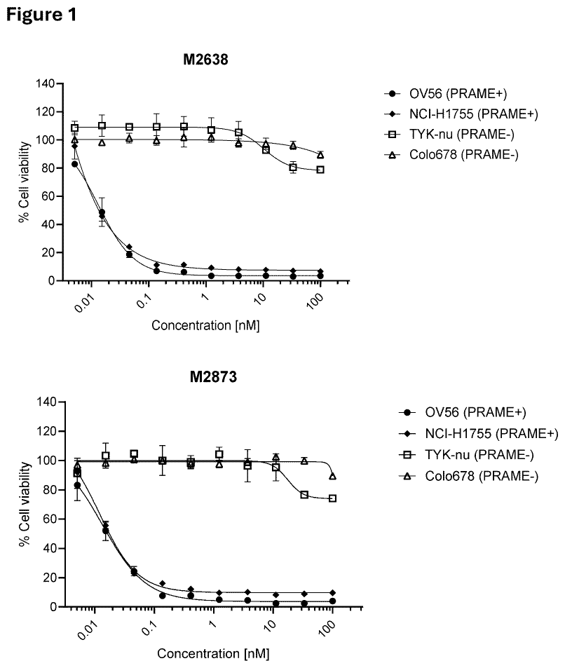

To reduce the complexity of the highly polymorphic restricting HLA genes, grouping different HLA molecules into clusters of several so-called HLA supertypes was suggested (see e.g., Wang M, Claesson M H. Classification of human leukocyte antigen (HLA) supertypes. Methods Mol Biol. 2014; 1184:309-17. Doi: 10.1007/978-1-4939-1115-8_17. PMID: 25048132; PMCID: PMC7121184). In each supertype, HLA molecules with similar peptide binding features are grouped into one supertype. It was hypothesized that if a peptide is able to bind to one allele within a supertype, it may also bind to all other alleles in this supertype. In practice, actually only a few peptides that are able to bind to one allele in a supertype can bind to all the other alleles within the supertype. In one aspect, the target peptides of the present invention are displayed by the A02 supertype (also termed A2-supertype), see for example FIG. 1 of Sidney et al., BMC Immunology, Vol. 9, Article number: 1 (2008). In certain embodiments, the target peptide is displayed by a serotype of the group consisting of HLA-A*02:01.

In one aspect, the invention provides antigen binding proteins that bind to MHC-displayed SLLQHLIGL (SEQ ID NO: 139), in particular antibody fragments such as scFvs and larger formats including such scFvs, e.g., Fab-scFvs and Fab-(scFvs)₂. Throughout the application, a pMHC may be described by its HLA and target peptide, for example HLA-A*02:01/PRAME is the SLLQHLIGL peptide of SEQ ID NO: 139 displayed on HLA-A*02:01. Similarly, any off-target peptide as described herein is presented by an HLA, such as HLA*02:01. The target pMHC complex wherein the peptide of SLLQHLIGL (SEQ ID NO: 139) is presented on an HLA-A*02:01 is herein also referred to as “HLA-A*02:01/PRAME antigen” or “HLA-A*02:01/PRAME”.

In certain embodiments, provided are isolated antigen binding proteins that bind to MHC-displayed SLLQHLIGL (SEQ ID NO: 139), i.e., the antigen binding protein is not associated or bound to the surface of a cell, such as a T cell. Isolated antigen binding proteins are separated from a component of its natural environment. In certain embodiments, the isolated antigen binding protein is purified to greater than 95% or 99% purity as determined by, for example, electrophoretic methods (e.g., SDS-PAGE, isoelectric focusing (IEF), capillary electrophoresis) or chromatography (e.g., ion exchange or reverse phase HPLC, affinity chromatography, size exclusion chromatography). In certain embodiments, the antigen binding protein is not a soluble TCR (e.g., a TCR lacking one or more of a transmembrane domain, an intracellular signaling domain, and constant domains). In certain embodiments, the antigen binding protein is or comprises a monoclonal antibody, in particular an antibody fragment such as a scFv.

As used herein, the term “PBS” refers to phosphate buffered saline. PBS is a pH-adjusted blend of phosphate buffers and saline solutions. In certain embodiments, the PBS comprises about 100-150 mM NaCl, about 1-5 mM KCl, about 1-10 mM Na₂HPO₄, and 1-5 mM KH₂PO₄. In certain embodiments, the PBS comprises 130 mM NaCl, 10 mM Na₂HPO₄, and pH of 6.0.

### Target Peptide-MHC and Antigen Binding Proteins Thereto

Described herein are antigen binding proteins, in particular isolated antibodies and artificial constructs comprising antibody fragments, that specifically recognize a target MHC-displayed SLLQHLIGL (SEQ ID NO: 139). As shown in the examples, the antigen binding proteins possess surprisingly high affinity while retaining high specificity for the target (i.e., low to no affinity for pMHCs displaying other targets, including off-target peptides, or beta-2-microglobulin).

In certain embodiments, the beta-2-microglobuin polypeptide comprises the amino acid sequence set forth in SEQ ID NO: 24.

The antigen binding proteins disclosed herein specifically recognize and bind to epitopes of a peptide/MHC complex (e.g., a PRAME/HLA complex, more specifically, a SLLQHLIGL (SEQ ID NO: 139)/HLA class I complex. In certain embodiments, the antigen binding proteins specifically bind a SLLQHLIGL (SEQ ID NO: 139)/HLA-A complex.

In certain embodiments, the MHC displaying the target peptide is of the HLA-A*02 supertype. In certain embodiments, the peptide is displayed by a serotype of the group consisting of HLA-A*02:01, HLA-A*02:02, HLA-A*02:03, HLA-A*02:04, HLA-A*02:05, HLA-A*02:06, HLA-A*02:07, HLA-A*02:14, HLA-A*02:17, HLA-A*68:02, and HLA-A*69:01, in particular HLA-A*02:01.

Safe therapeutics require a high selectivity for the target peptide as reactivity to off-target peptides is of concern. In some embodiments, an off-target peptide has no more than 2, 3, 4, or 5 amino acid mismatches from SLLQHLIGL (SEQ ID NO: 139). In certain embodiments, the off-target peptide is selected from any one of SEQ ID NOs.: 148-203 (i.e., a sequence selected from Table 5 and/or Table 6).

In certain embodiments, the antigen binding protein specifically binds to SLLQHLIGL (SEQ ID NO: 139) displayed by HLA-A*02 on the surface of a cell, such as a cancer cell. In certain embodiments, the cell is a T2 cell expressing HLA-A*02 that has been pulsed with the target peptide SLLQHLIGL (SEQ ID NO: 139). In another embodiment, the cell is a Cos-7 cell expressing HLA-A*02 that has been pulsed with the target peptide SLLQHLIGL (SEQ ID NO: 139). In yet other embodiments, the cell is a cancer cell that endogenously presents the target peptide SLLQHLIGL (SEQ ID NO: 139) by an HLA complex (i.e., without pulsing).

In certain embodiments, the antigen binding proteins described herein comprises cytotoxic activity against an MHC-displaying SLLQHLIGL (SEQ ID NO: 139) cell. In certain embodiments, the antigen binding comprises at least one additional binding domain targeting an immune cell. In certain embodiments, the antigen binding protein lacks detectable cytotoxic activity against a non-SLLQHLIGL (SEQ ID NO: 139) pMHC presenting cell.

In certain embodiments, the antigen binding protein is not a T cell receptor (TCR).

In certain embodiments, the antigen binding protein of the disclosure has an affinity (K_{D}) to an MHC-displayed SLLQHLIGL (SEQ ID NO: 139) of at least about 5 nM or stronger (e.g., at least about 3 nM, about 2 nM, about 1.5 nM, about 1.2 nM, about 1 nM, about 0.9 nM, about 0.75 nM, about 0.5 nM, about 0.25 nM, about 0.1 nM, about 0.09 nM, about 0.08 nM, about 0.07 nM, about 0.06 nM, about 0.04 nM, or about 0.02 nM), as determined by SPR. In certain embodiments, the antigen binding protein has an affinity (K_{D}) to an MHC-displayed SLLQHLIGL (SEQ ID NO: 139) of about 0.04-0.16 nM, more particularly 0.07-0.09 nM, as determined by SPR.

In certain embodiments, the antigen binding protein of the disclosure shows at least a 5-fold decrease in affinity when one or more of amino acid positions of the group consisting of H5, L6, 17, G8 and L9 of SLLQHLIGL (SEQ ID NO: 139) are substituted with alanine. In certain embodiments, the antigen binding protein shows at least a 10-fold decrease in affinity when one or more of amino acid positions of the group consisting of H5, L6, 17, G8 and L9 of SLLQHLIGL (SEQ ID NO: 139) are substituted with alanine.

In certain embodiments, the antigen binding protein of the disclosure shows at least a 5-fold decrease in affinity when one or more of amino acid positions of the group consisting of L3, H5, L6, 17, G8 and L9 of SLLQHLIGL (SEQ ID NO: 139), are substituted with arginine. In certain embodiments, the antigen binding protein shows at least a 10-fold decrease in affinity when one or more of amino acid positions of the group consisting of L3, H5, L6, 17, G8 and L9 of SLLQHLIGL (SEQ ID NO: 139), are substituted with arginine.

In certain embodiments, the antigen binding protein of the disclosure has at least a 5-fold decrease in affinity when one or more of amino acid positions of the group consisting of L2, L6, 17, G8 and L9 of SLLQHLIGL (SEQ ID NO: 139), are substituted with aspartic acid. In certain embodiments, the antigen binding protein shows at least a 10-fold decrease in affinity when one or more of amino acid positions of the group consisting of L2, L6, 17, G8 and L9 of SLLQHLIGL (SEQ ID NO: 139), are substituted with aspartic acid.

In certain embodiments, the antigen binding protein of the disclosure is highly selective and shows a significant reduction in affinity to a HLA-restricted off-target peptide, compared to HLA-restricted SLLQHLIGL (SEQ ID NO: 139). Off-target peptides expressed in healthy tissues may pose a cross-reactivity risk. In some embodiments, said HLA-restricted off-target peptide is derived from the gene product of HDAC5, LRRC70, DDX60L, ZNF318, IFIT1, VPS13B, CCDC51, TMED9, GIMAP8, KCNG3, TSC22D2, TUBG2, BBS10, and/or RALGAPB. In some embodiments, said HLA-restricted off-target peptide is derived from the group consisting of SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 198, SEQ ID NO: 199, SEQ ID NO: 200, SEQ ID NO: 201, SEQ ID NO: 202 and/or SEQ ID NO: 203. In certain embodiments, said reduction in affinity is at least about 20-fold (e.g., about 25-fold, 30-fold, 40-fold, 50-fold, 75-fold, 80-fold, 90-fold, 100-fold or a higher decrease in affinity).

In certain embodiments, the antigen binding protein of the disclosure comprises: (a) an antibody heavy chain variable (VH) domain comprising an HCDR1 amino acid sequence of STYGVS (SEQ ID NO: 25), an HCDR2 amino acid sequence of YIDPVYGSTX₁YAX₂×₃VX₄G (SEQ ID NO: 26), wherein X₁ is Yor H, X₂ is S or D, X₃ is W or S and X₄ is N or K and an HCDR3 amino acid sequence of DLYAGSSGYYXIYSL (SEQ ID NO: 27), wherein X₅ is M or V; and (b) an antibody light chain variable (VL) domain comprising an LCDR1 amino acid sequence of X₆SSQSVYNNLLG (SEQ ID NO: 28), wherein X₆ is Q or R, an LCDR2 amino acid sequence of SASTX₇AS (SEQ ID NO: 29), wherein X₇ is L or R, and an LCDR3 amino acid sequence of QGTYYX₈GDWYYP (SEQ ID NO: 30), wherein X₈ is N or T.

In certain embodiments, the antigen binding protein of the disclosure comprises: (a) an antibody heavy chain variable (VH) domain comprising a HCDR1 amino acid sequence of STYGVS (SEQ ID NO: 25), a HCDR2 amino acid sequence selected from YIDPVYGSTYYASWVNG (SEQ ID NO: 32) and YIDPVYGSTHYADSVKG (SEQ ID NO: 38), a HCDR3 amino acid sequence selected from DLYAGSSGYYMIYSL (SEQ ID NO: 33) and DLYAGSSGYYVIYSL (SEQ ID NO: 51); and (b) an antibody light chain variable (VL) domain comprising an LCDR1 amino acid sequence selected from QSSQSVYNNLLG (SEQ ID NO: 34) and RSSQSVYNNLLG (SEQ ID NO: 46), a LCDR2 amino acid sequence selected from SASTLAS (SEQ ID NO: 41) and SASTRAS (SEQ ID NO: 59), and a LCDR3 amino acid sequence selected from QGTYYNGDWYYP (SEQ ID NO: 36) and QGTYYTGDWYYP (SEQ ID NO: 48).

In certain embodiments, the antigen binding protein of the disclosure comprises (a) an antibody heavy chain variable (VH) domain comprising an HCDR1 amino acid sequence of STYGVS (SEQ ID NO: 31), an HCDR2 amino acid sequence of YIDPVYGSTYYASWVNG (SEQ ID NO: 32), and an HCDR3 amino acid sequence of DLYAGSSGYYMIYSL (SEQ ID NO: 33); and (b) an antibody light chain variable (VL) domain comprising an LCDR1 amino acid sequence of QSSQSVYNNLLG (SEQ ID NO: 34), an LCDR2 amino acid sequence of SASTLAS (SEQ ID NO: 35), and an LCDR3 amino acid sequence of QGTYYNGDWYYP (SEQ ID NO: 36).

In certain embodiments, the antigen binding protein of the disclosure comprises (a) an antibody heavy chain variable (VH) domain comprising an HCDR1 amino acid sequence of STYGVS (SEQ ID NO: 37), an HCDR2 amino acid sequence of YIDPVYGSTHYADSVKG (SEQ ID NO: 38), and an HCDR3 amino acid sequence of DLYAGSSGYYMIYSL (SEQ ID NO: 39); and (b) an antibody light chain variable (VL) domain comprising an LCDR1 amino acid sequence of RSSQSVYNNLLG (SEQ ID NO: 40), an LCDR2 amino acid sequence of SASTLAS (SEQ ID NO: 41), and an LCDR3 amino acid sequence of QGTYYNGDWYYP (SEQ ID NO: 42).

In certain embodiments, the antigen binding protein of the disclosure comprises (a) an antibody heavy chain variable (VH) domain comprising an HCDR1 amino acid sequence of STYGVS (SEQ ID NO: 43), an HCDR2 amino acid sequence of YIDPVYGSTHYADSVKG (SEQ ID NO: 44), and an HCDR3 amino acid sequence of DLYAGSSGYYMIYSL (SEQ ID NO: 45); and (b) an antibody light chain variable (VL) domain comprising an LCDR1 amino acid sequence of RSSQSVYNNLLG (SEQ ID NO: 46), an LCDR2 amino acid sequence of SASTLAS (SEQ ID NO: 47), and an LCDR3 amino acid sequence of QGTYYTGDWYYP (SEQ ID NO: 48).

In certain embodiments, the antigen binding protein of the disclosure comprises (a) an antibody heavy chain variable (VH) domain comprising an HCDR1 amino acid sequence of STYGVS (SEQ ID NO: 49), an HCDR2 amino acid sequence of YIDPVYGSTHYADSVKG (SEQ ID NO: 50), and an HCDR3 amino acid sequence of DLYAGSSGYYVIYSL (SEQ ID NO: 51); and (b) an antibody light chain variable (VL) domain comprising an LCDR1 amino acid sequence of RSSQSVYNNLLG (SEQ ID NO: 52), an LCDR2 amino acid sequence of SASTLAS (SEQ ID NO: 53), and an LCDR3 amino acid sequence of QGTYYTGDWYYP (SEQ ID NO: 54).

In certain embodiments, the antigen binding protein of the disclosure comprises (a) an antibody heavy chain variable (VH) domain comprising an HCDR1 amino acid sequence of STYGVS (SEQ ID NO: 55), an HCDR2 amino acid sequence of YIDPVYGSTHYADSVKG (SEQ ID NO: 56), and an HCDR3 amino acid sequence of DLYAGSSGYYMIYSL (SEQ ID NO: 57); and (b) an antibody light chain variable (VL) domain comprising an LCDR1 amino acid sequence of RSSQSVYNNLLG (SEQ ID NO: 58), an LCDR2 amino acid sequence of SASTRAS (SEQ ID NO: 59), and an LCDR3 amino acid sequence of QGTYYTGDWYYP (SEQ ID NO: 60).

In certain embodiments, the antigen binding protein of the disclosure is a variant thereof comprising 1, 2, 3, 4, 5, or 6 substitutions in any one or more of the HCDR1, HCDR2, HCDR3, LCDR1, LCDR2 and LCDR3. In certain embodiments, the substitution is in LCDR1, LCDR2 and/or LCDR3 at a position selected from the group consisting of 24, 54, and 94 (according to Kabat numbering). In certain embodiments, the substitution is in a light chain CDR and selected from the group consisting of Q24R, R24Q, L54R, R54L, N94T or T94N (according to Kabat numbering). In certain embodiments, the substitution is in HCDR1, HCDR2 and/or HCDR3 at a position selected from the group consisting of 58, 61, 62, 64 and 100e (according to Kabat numbering). In certain embodiments, the substitution is in a heavy chain CDR and selected from the group consisting of Y58H, H58Y, D61S, S61D, W62S, S62W, N64K, K64N, M100 eV and V100eM (according to Kabat numbering).

In certain embodiments, the antigen binding protein comprises an antibody heavy chain variable (VH) domain comprising one or more framework sequences HFR1, HFR2, HFR3 and/or HFR4 selected from the group consisting of SEQ ID NO: 69, SEQ ID NO: 62, SEQ ID NO: 63 and/or SEQ ID NO: 64 or respective variants thereof comprising 1, 2, 3, 4, 5, or 6 substitutions. In certain embodiments, the HFR1 variant is selected from the group consisting of SEQ ID NOs: 61, 204 or 205, In certain embodiments, the HFR3 variant is SEQ ID NO: 206. In certain embodiments, said framework sequences HFR1, HFR2, HFR3 and/or HFR4 comprise or consists of SEQ ID NOs: 61, 62, 63 and/or 64. In certain embodiments, said framework sequences HFR1, HFR2, HFR3 and/or HFR4 comprise or consists of SEQ ID NOs: 204, 62, 63 and/or 64. In certain embodiments, said framework sequences HFR1, HFR2, HFR3 and/or HFR4 comprise or consists of SEQ ID NOs: 204, 62, 206 and/or 64. In certain embodiments, said framework sequences HFR1, HFR2, HFR3 and/or HFR4 comprise or consists of SEQ ID NOs: 205, 62, 63 and/or 64.

In certain embodiments, the antigen binding protein comprises an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 67 and/or SEQ ID NO: 68 or respective variants thereof comprising 1, 2, 3, 4, 5, or 6 substitutions. One exemplary LFR3 variant is SEQ ID NO: 70. In certain embodiments, the antigen binding protein comprises an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 70 and/or SEQ ID NO: 68 or respective variants thereof comprising 1, 2, 3, 4, 5, or 6 substitutions.

In certain embodiments, the antigen binding protein comprises (1) an antibody heavy chain variable (VH) domain comprising one or more framework sequences HFR1, HFR2, HFR3 and/or HFR4 selected from the group consisting of SEQ ID NO: 61, SEQ ID NO: 62, SEQ ID NO: 63 and/or SEQ ID NO: 64 or respective variants thereof comprising 1, 2, 3, 4, 5, or 6 substitutions; and (2) an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 67 and/or SEQ ID NO: 68 or respective variants thereof comprising 1, 2, 3, 4, 5, or 6 substitutions.

In certain embodiments, the antigen binding protein comprises (1) an antibody heavy chain variable (VH) domain comprising one or more framework sequences HFR1, HFR2, HFR3 and/or HFR4 selected from the group consisting of SEQ ID NO: 61, SEQ ID NO: 62, SEQ ID NO: 63 and/or SEQ ID NO: 64 or respective variants thereof comprising 1, 2, 3, 4, 5, or 6 substitutions; and (2) an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 70 and/or SEQ ID NO: 68 or respective variants thereof comprising 1, 2, 3, 4, 5, or 6 substitutions.

In certain embodiments, the antigen binding protein comprises an antibody heavy chain variable (VH) domain comprising one or more framework sequences HFR1, HFR2, HFR3 and/or HFR4 selected from the group consisting of SEQ ID NO: 69, SEQ ID NO: 62, SEQ ID NO: 63 and/or SEQ ID NO: 64 or respective variants thereof comprising 1, 2, 3, 4, 5, or 6 substitutions; and (2) an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 67 and/or SEQ ID NO: 68 or respective variants thereof comprising 1, 2, 3, 4, 5, or 6 substitutions.

In certain embodiments, the antigen binding protein comprises an antibody heavy chain variable (VH) domain comprising one or more framework sequences HFR1, HFR2, HFR3 and/or HFR4 selected from the group consisting of SEQ ID NO: 69, SEQ ID NO: 62, SEQ ID NO: 63 and/or SEQ ID NO: 64 or respective variants thereof comprising 1, 2, 3, 4, 5, or 6 substitutions; and (2) an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 70 and/or SEQ ID NO: 68 or respective variants thereof comprising 1, 2, 3, 4, 5, or 6 substitutions.

In certain embodiments, the antigen binding protein comprises:
(1) an antibody heavy chain variable (VH) domain comprising the amino acid sequence of SEQ ID NO: 71 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 72 or a variant thereof that is at least about 75%, 78%, 79%, 80%, 81%, 83%, 85%, 90%, 95%, 96%, 97%, 98% or 99% identical thereto;
(2) an antibody heavy chain variable (VH) domain comprising the amino acid sequence of SEQ ID NO: 73 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 74 or a variant thereof that is at least 75%, 78%, 79%, 80%, 81%, 83%, 85%, 90%, 95%, 96%, 97%, 98% or 99% identical thereto;
(3) an antibody heavy chain variable (VH) domain comprising the amino acid sequence of SEQ ID NO: 75 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 76 or a variant thereof that is at least about 75%, 78%, 79%, 80%, 81%, 83%, 85%, 90%, 95%, 96%, 97%, 98% or 99% identical thereto;
(4) an antibody heavy chain variable (VH) domain comprising the amino acid sequence of SEQ ID NO: 77 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 78 or a variant thereof that is at least about 75%, 78%, 79%, 80%, 81%, 83%, 85%, 90%, 95%, 96%, 97%, 98% or 99% identical thereto;
(5) an antibody heavy chain variable (VH) domain comprising the amino acid sequence of SEQ ID NO: 79 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 80 or a variant thereof that is at least about 75%, 78%, 79%, 80%, 81%, 83%, 85%, 90%, 95%, 96%, 97%, 98% or 99% identical thereto;
(6) an antibody heavy chain variable (VH) domain comprising the amino acid sequence of SEQ ID NO: 81 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 82 or a variant thereof that is at least about 75%, 78%, 79%, 80%, 81%, 83%, 85%, 90%, 95%, 96%, 97%, 98% or 99% identical thereto;
(7) an antibody heavy chain variable (VH) domain comprising the amino acid sequence of SEQ ID NO: 83 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 84 or a variant thereof that is at least about 75%, 78%, 79%, 80%, 81%, 83%, 85%, 90%, 95%, 96%, 97%, 98% or 99% identical thereto;
(8) an antibody heavy chain variable (VH) domain comprising the amino acid sequence of SEQ ID NO: 85 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 86 or a variant thereof that is at least about 75%, 78%, 79%, 80%, 81%, 83%, 85%, 90%, 95%, 96%, 97%, 98% or 99% identical thereto;
(9) an antibody heavy chain variable (VH) domain comprising the amino acid sequence of SEQ ID NO: 87 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 88 or a variant thereof that is at least about 75%, 78%, 79%, 80%, 81%, 83%, 85%, 90%, 95%, 96%, 97%, 98% or 99% identical thereto;
(10) an antibody heavy chain variable (VH) domain comprising the amino acid sequence of SEQ ID NO: 89 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 90 or a variant thereof that is at least about 75%, 78%, 79%, 80%, 81%, 83%, 85%, 90%, 95%, 96%, 97%, 98% or 99% identical thereto; or
(11) an antibody heavy chain variable (VH) domain comprising the amino acid sequence of SEQ ID NO: 91 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 92 or a variant thereof that is at least about 75%, 78%, 79%, 80%, 81%, 83%, 85%, 90%, 95%, 96%, 97%, 98% or 99% identical thereto.

In certain embodiments, the variant of said VH and or VL amino acid sequences comprises substitutions, such as 1, 2, 3, 4, 5, 6, 7, 8, 9 or more substitutions, within the VH and/or VL domain. In certain embodiments, such substitutions are within the CDRs. In certain embodiments, such substitutions are within the framework regions. In certain embodiments, the variant comprises one or more substitutions in the VL at Kabat positions 24, 54, 83 and/or 94. In certain embodiments, a VL substitution is selected from the group consisting of Q24R, R24Q, L54R, R54L, F83S, S83F, T94N and/or N94T (according to Kabat numbering). In other embodiments, the variant comprises one or more substitutions in the VL at Kabat position 1, 2, 3, 7, 11, 14, 17, 18, 22, 24, 42, 43, 54, 70, 77, 78, 80, 81, 83, 85, 94, 100, 103, 105 and/or 106. In certain embodiments the variant comprises one or more substitutions in the VH at Kabat positions 1, 16, 28, 29, 16, 58, 61, 62, 64, 93 and/or 100e. In certain embodiments, a VH substitution is selected from the group consisting of E1Q, Q1E, R16G, G16R, D28T, T28D, F29V, V29F, G16R, R16G, Y58H, H58Y, S61D, D61S, W62S, S62W, N64K, K64N, V93A A93V, V100eM, and/or M100 eV (according to Kabat numbering). In other embodiments, the variant comprises one or more substitutions in the VH at Kabat position 1, 2, 11, 16, 19, 23, 28, 29, 44, 58, 61, 62, 64, 71, 72, 74, 75, 82, 83, 85, 89, 91, 93, 100e, 105 and/or 108.

In certain embodiments, the antigen binding protein is or comprises a full-length immunoglobulin or an antibody fragment such as a Fab, a Fab′, a F(ab′)₂, a scFv, a Fv fragment.

In certain embodiments, the antigen binding protein comprises a scFv with an amino acid sequence that is at least about 79%, 80%, 85%, 90%, 95%, 96%, 97%, 98%, 99% or 100% identical to an amino acid sequence selected from the group consisting of SEQ ID NOs: 93-103.

In certain embodiments, the antigen binding protein is a variant of said scFvs, comprising substitutions, such as 1, 2, 3, 4, 5, 6, 7, 8, 9, 10, 11, 12, 13, 14, 15, 16, 17, 18, 19, 20 or more substitutions, within the VH domain, the VL domain and/or the linker. In certain embodiments, such substitutions are within the CDRs. In certain embodiments, such substitutions are within the framework regions of the VH or the VL domain.

TABLE 2
The sequences disclosed herein are:
Linkers

| | |
|---|---|
| SEQ ID NO: 1 | (GGGGS)ₙ wherein n = 1, 2, 3, 4 or 5 |
| SEQ ID NO: 2 | GGGGS |
| SEQ ID NO: 3 | GGGGSGGGGS |
| SEQ ID NO: 4 | GGGGSGGGGSGGGGS |
| SEQ ID NO: 5 | GGGGSGGGGSGGGGSGGGGS |
| SEQ ID NO: 6 | GGGGGSGGGGSGGGGSGGGGS |
| SEQ ID NO: 7 | GGGGSGGGGSGGGGSGGGGAS |
| SEQ ID NO: 22 | DGGGS |
| SEQ ID NO: 8 | TGEKP |
| SEQ ID NO: 9 | GGRR |
| SEQ ID NO: 10 | EGKSSGSGSESKVD |
| SEQ ID NO: 11 | KESGSVSSEQLAQFRSLD |
| SEQ ID NO: 12 | GGRRGGGS |
| SEQ ID NO: 13 | LRQRDGERP |
| SEQ ID NO: 14 | LRQKDGGGSERP |
| SEQ ID NO: 15 | GSTSGSGKPGSGEGSTKG |
| SEQ ID NO: 16 | EPKSC |
| SEQ ID NO: 17 | EPKSCGGGGS |
| SEQ ID NO: 18 | EPKSCDKTHT |
| SEQ ID NO: 19 | EPKSCDKTHTGGGGS |
| SEQ ID NO: 20 | DKTHT |
| SEQ ID NO: 21 | DKTHTGGGGS |

MHC related sequences

| | | |
|---|---|---|
| SEQ ID NO: 23 | HLA-A*02:01 | GSHSMRYFFTSVSRPGRGEPRFIAVGYVDD |
| | extracellular | TQFVRFDSDAASQRMEPRAPWIEQEGPEY |
| | domain | WDGETRKVKAHSQTHRVDLGTLRGYYNQ |
| | | SEAGSHTVQRMYGCDVGSDWRFLRGYHQ |
| | | YAYDGKDYIALKEDLRSWTAADMAAQTT |
| | | KHKWEAAHVAEQLRAYLEGTCVEWLRRY |
| | | LENGKETLQRTDAPKTHMTHHAVSDHEAT |
| | | LRCWALSFYPAEITLTWQRDGEDQTQDTEL |
| | | VETRPAGDGTFQKWAAVVVPSGQEQRYTC |
| | | HVQHEGLPKPLTLRWE |
| SEQ ID NO: 24 | human ß2m | IQRTPKIQVYSRHPAENGKSNFLNCYVSGFH |
| | | PSDIEVDLLKNGERIEKVEHSDLSFSKDWSF |
| | | YLLYYTEFTPTEKDEYACRVNHVTLSQPKI |
| | | VKWDRDM |

pMHC Antigen binding protein related sequences

| | | |
|---|---|---|
| SEQ ID NO: 25 | HCDR1 | STYGVS |
| SEQ ID NO: 26 | HCDR2 | YIDPVYGSTX₁YAX₂X₃VX₄G |
| | | Wherein |
| | | X₁ is Yor H |
| | | X₂ is S or D |
| | | X₃ is W or S |
| | | X₄ is N or K |
| SEQ ID NO: 27 | HCDR3 | DLYAGSSGYYX₅IYSL |
| | | Wherein X₅ is M or V |
| SEQ ID NO: 28 | LCDR1 | X₆SSQSVYNNLLG |
| | | Wherein X₆ is Q or R |
| SEQ ID NO: 29 | LCDR2 | SASTX₇AS |
| | | Wherein X₇ is L or R |
| SEQ ID NO: 30 | LCDR3 | QGTYYX₈GDWYYP |
| | | Wherein X₈ is N or T |
| SEQ ID NO: 31 | M2638, M2913 | STYGVS |
| | HCDR1 | |
| SEQ ID NO: 32 | M2638, M2913 | YIDPVYGSTYYASWVNG |
| | HCDR2 | |
| SEQ ID NO: 33 | M2638, M2913 | DLYAGSSGYYMIYSL |
| | HCDR3 | |
| SEQ ID NO: 34 | M2638, M2913 | QSSQSVYNNLLG |
| | LCDR1 | |
| SEQ ID NO: 35 | M2638, M2913 | SASTLAS |
| | LCDR2 | |
| SEQ ID NO: 36 | M2638, M2913 | QGTYYNGDWYYP |
| | LCDR3 | |
| SEQ ID NO: 37 | M3076 HCDR1 | STYGVS |
| SEQ ID NO: 38 | M3076 HCDR2 | YIDPVYGSTHYADSVKG |
| SEQ ID NO: 39 | M3076 HCDR3 | DLYAGSSGYYMIYSL |
| SEQ ID NO: 40 | M3076 LCDR1 | RSSQSVYNNLLG |
| SEQ ID NO: 41 | M3076 LCDR2 | SASTLAS |
| SEQ ID NO: 42 | M3076 LCDR3 | QGTYYNGDWYYP |
| SEQ ID NO: 43 | M3182, M3202, | STYGVS |
| | M3283, M3284, | |
| | M3285, M3286 | |
| | HCDR1 | |
| SEQ ID NO: 44 | M3182, M3202, | YIDPVYGSTHYADSVKG |
| | M3283, M3284, | |
| | M3285, M3286 | |
| | HCDR2 | |
| SEQ ID NO: 45 | M3182, M3202, | DLYAGSSGYYMIYSL |
| | M3283, M3284, | |
| | M3285, M3286 | |
| | HCDR3 | |
| SEQ ID NO: 46 | M3182, M3202, | RSSQSVYNNLLG |
| | M3283, M3284, | |
| | M3285, M3286 | |
| | LCDR1 | |
| SEQ ID NO: 47 | M3182, M3202, | SASTLAS |
| | M3283, M3284, | |
| | M3285, M3286 | |
| | LCDR2 | |
| SEQ ID NO: 48 | M3182, M3202, | QGTYYTGDWYYP |
| | M3283, M3284, | |
| | M3285, M3286 | |
| | LCDR3 | |
| SEQ ID NO: 49 | M3287 HCDR1 | STYGVS |
| SEQ ID NO: 50 | M3287 HCDR2 | YIDPVYGSTHYADSVKG |
| SEQ ID NO: 51 | M3287 HCDR3 | DLYAGSSGYYVIYSL |
| SEQ ID NO: 52 | M3287 LCDR1 | RSSQSVYNNLLG |
| SEQ ID NO: 53 | M3287 LCDR2 | SASTLAS |
| SEQ ID NO: 54 | M3287 LCDR3 | QGTYYTGDWYYP |
| SEQ ID NO: 55 | M3293 HCDR1 | STYGVS |
| SEQ ID NO: 56 | M3293 HCDR2 | YIDPVYGSTHYADSVKG |
| SEQ ID NO: 57 | M3293 HCDR3 | DLYAGSSGYYMIYSL |
| SEQ ID NO: 58 | M3293 LCDR1 | RSSQSVYNNLLG |
| SEQ ID NO: 59 | M3293 LCDR2 | SASTRAS |
| SEQ ID NO: 60 | M3293 LCDR3 | QGTYYTGDWYYP |
| SEQ ID NO: 61 | HFR1 | EVQLVESGGGSVQPGGSLRLSCAASGFDF |
| SEQ ID NO: 62 | HFR2 | WVRQAPGKCLEWIG |
| SEQ ID NO: 63 | HFR3 | RFTISRDNSKNTLYLQMNSLRAEDTASYYCVR |
| SEQ ID NO: 64 | HFR4 | WGQGTSVTVSS |
| SEQ ID NO: 65 | LFR1 | DIQMTQSPSSLSASVGDRVTITC |
| SEQ ID NO: 66 | LFR2 | WYQQKPGKAPKLLIY |
| SEQ ID NO: 67 | LFR3 | GVPSRFSGSGSGTDFTLTISSLQPEDFATYYC |
| SEQ ID NO: 68 | LFR4 | FGCGTKVEIK |
| SEQ ID NO: 69 | HFR1 | QVQLVESGGGSVQPGRSLRLSCAASGFDF |
| SEQ ID NO: 204 | HFR1 | QVQLVESGGGSVQPGRSLRLSCAASGFTF |
| SEQ ID NO: 205 | HFR1 | QVQLVESGGGSVQPGRSLRLSCAASGFTV |
| SEQ ID NO: 206 | HFR3 | RFTISRDNSKNTLYLQMNSLRAEDTASYYCAR |
| SEQ ID NO: 70 | LFR3 | GVPSRFSGSGSGTDFTLTISSLQPEDSATYYC |
| SEQ ID NO: 71 | M2638 VH | QEQLVESGGGLVQPGGSLKLSCKASGFDFS |
| | | TYGVSWVRQAPGKGLEWIGYIDPVYGSTY |
| | | YASWVNGRFTISSHNAQNTLYLQLNSLTAA |
| | | DTATYFCVRDLYAGSSGYYMIYSLWGPGT |
| | | LVTVSS |
| SEQ ID NO: 72 | M2638 VL | ELVMTQTPSSVSAAVGGTVTIKCQSSQSVY |
| | | NNLLGWYQQKPGQPPKLLIYSASTLASGVP |
| | | SRFSGSGSGTEFTLTISGVQADDAASYYCQ |
| | | GTYYNGDWYYPFGGGTEVVVK |
| SEQ ID NO: 73 | M2913 VH | EVQLVESGGGSVQPGGSLRLSCAASGFDFS |
| | | TYGVSWVRQAPGKCLEWIGYIDPVYGSTY |
| | | YASWVNGRFTISRDNSKNTLYLQMNSLRA |
| | | EDTASYYCVRDLYAGSSGYYMIYSLWGQG |
| | | TSVTVSS |
| SEQ ID NO: 74 | M2913 VL | DIQMTQSPSSLSASVGDRVTITCQSSQSVYN |
| | | NLLGWYQQKPGKAPKLLIYSASTLASGVPS |
| | | RFSGSGSGTDFTLTISSLOPEDFATYYCQGT |
| | | YYNGDWYYPFGCGTKVEIK |
| SEQ ID NO: 75 | M3076 VH | QVQLVESGGGSVQPGRSLRLSCAASGFDFS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYMIYSLWGQGT |
| | | SVTVSS |
| SEQ ID NO: 76 | M3076 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDFATYYCQGT |
| | | YYNGDWYYPFGCGTKVEIK |
| SEQ ID NO: 77 | M3182 VH | QVQLVESGGGSVQPGRSLRLSCAASGFDFS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYMIYSLWGQGT |
| | | SVTVSS |
| SEQ ID NO: 78 | M3182 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDFATYYCQGT |
| | | YYTGDWYYPFGCGTKVEIK |
| SEQ ID NO: 79 | M3202 VH | QVQLVESGGGSVQPGRSLRLSCAASGFDFS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYMIYSLWGQGT |
| | | SVTVSS |
| SEQ ID NO: 80 | M3202 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIK |
| SEQ ID NO: 81 | M3283 VH | QVQLVESGGGSVQPGRSLRLSCAASGFTFS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYMIYSLWGQGT |
| | | SVTVSS |
| SEQ ID NO: 82 | M3283 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIK |
| SEQ ID NO: 83 | M3284 VH | QVQLVESGGGSVQPGRSLRLSCAASGFTVS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYMIYSLWGQGT |
| | | SVTVSS |
| SEQ ID NO: 84 | M3284 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIK |
| SEQ ID NO: 85 | M3285 VH | QVQLVESGGGSVQPGRSLRLSCAASGFDES |
| | | TYGVSWVRQAPGKCLEWIGYIDPVYGSTH |
| | | YADSVKGRFTISRDNSKNTLYLQMNSLRAE |
| | | DTASYYCARDLYAGSSGYYMIYSLWGQGT |
| | | SVTVSS |
| SEQ ID NO: 86 | M3285 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIK |
| SEQ ID NO: 87 | M3286 VH | VQPGRSLRLSCAASGFTFSTYGVSWVRQAP |
| | | GKCLEWIGYIDPVYGSTHYADSVKGRFTIS |
| | | RDNSKNTLYLQMNSLRAEDTASYYCARDL |
| | | YAGSSGYYMIYSLWGQGTSVTVSS |
| SEQ ID NO: 88 | M3286 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIK |
| SEQ ID NO: 89 | M3287 VH | QVQLVESGGGSVQPGRSLRLSCAASGFDFS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYVIYSLWGQGT |
| | | SVTVSS |
| SEQ ID NO: 90 | M3287 VL | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIK |
| SEQ ID NO: 91 | M3293 VH | QVQLVESGGGSVQPGRSLRLSCAASGFDFS |
| | | TYGVSWVRQAPGKCLEWIGYIDPVYGSTH |
| | | YADSVKGRFTISRDNSKNTLYLQMNSLRAE |
| | | DTASYYCVRDLYAGSSGYYMIYSLWGQGT |
| | | SVTVSS |
| SEQ ID NO: 92 | M3293 VL | DIQMTQSPSSLSASVGDRVTITCRSSQSVYN |
| | | NLLGWYQQKPGKAPKLLIYSASTRASGVPS |
| | | RFSGSGSGTDFTLTISSLQPEDSATYYCQGT |
| | | YYTGDWYYPFGCGTKVEIK |
| SEQ ID NO: 93 | scFv M2638 | QEQLVESGGGLVQPGGSLKLSCKASGFDFS |
| | | TYGVSWVRQAPGKGLEWIGYIDPVYGSTY |
| | | YASWVNGRFTISSHNAQNTLYLQLNSLTAA |
| | | DTATYFCVRDLYAGSSGYYMIYSLWGPGT |
| | | LVTVSSGGGGSGGGGSGGGGSGGGGSELV |
| | | MTQTPSSVSAAVGGTVTIKCQSSQSVYNNL |
| | | LGWYQQKPGQPPKLLIYSASTLASGVPSRFS |
| | | GSGSGTEFTLTISGVQADDAASYYCQGTYY |
| | | NGDWYYPFGGGTEVVVK |
| SEQ ID NO: 94 | scFv M2913 | DIQMTQSPSSLSASVGDRVTITCQSSQSVYN |
| | | NLLGWYQQKPGKAPKLLIYSASTLASGVPS |
| | | RFSGSGSGTDFTLTISSLQPEDFATYYCQGT |
| | | YYNGDWYYPFGCGTKVEIKGGGGSGGGGS |
| | | GGGGSGGGGSEVQLVESGGGSVQPGGSLR |
| | | LSCAASGFDFSTYGVSWVRQAPGKCLEWIG |
| | | YIDPVYGSTYYASWVNGRFTISRDNSKNTL |
| | | YLQMNSLRAEDTASYYCVRDLYAGSSGYY |
| | | MIYSLWGQGTSVTVSS |
| SEQ ID NO: 95 | scFv M3076 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDFATYYCQGT |
| | | YYNGDWYYPFGCGTKVEIKGGGGSGGGGS |
| | | GGGGSGGGGSQVQLVESGGGSVQPGRSLR |
| | | LSCAASGFDFSTYGVSWVRQAPGKCLEWIG |
| | | YIDPVYGSTHYADSVKGRFTISRDNSKNTL |
| | | YLQMNSLRAEDTASYYCVRDLYAGSSGYY |
| | | MIYSLWGQGTSVTVSS |
| SEQ ID NO: 96 | scFv M3182 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDFATYYCQGT |
| | | YYTGDWYYPFGCGTKVEIKGGGGSGGGGS |
| | | GGGGSGGGGSQVQLVESGGGSVQPGRSLR |
| | | LSCAASGFDFSTYGVSWVRQAPGKCLEWIG |
| | | YIDPVYGSTHYADSVKGRFTISRDNSKNTL |
| | | YLQMNSLRAEDTASYYCVRDLYAGSSGYY |
| | | MIYSLWGQGTSVTVSS |
| SEQ ID NO: 97 | scFv 3202 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFDFSTYGVSWVRQAPGKCLEWIG |
| | | YIDPVYGSTHYADSVKGRFTISRDNSKNTL |
| | | YLQMNSLRAEDTASYYCVRDLYAGSSGYY |
| | | MIYSLWGQGTSVTVSS |
| SEQ ID NO: 98 | scFv M3283 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFTFSTYGVSWVRQAPGKCLEWIG |
| | | YIDPVYGSTHYADSVKGRFTISRDNSKNTL |
| | | YLQMNSLRAEDTASYYCVRDLYAGSSGYY |
| | | MIYSLWGQGTSVTVSS |
| SEQ ID NO: 99 | scFv M3284 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFTVSTYGVSWVRQAPGKCLEWI |
| | | GYIDPVYGSTHYADSVKGRFTISRDNSKNT |
| | | LYLQMNSLRAEDTASYYCVRDLYAGSSGY |
| | | YMIYSLWGQGTSVTVSS |
| SEQ ID NO: 100 | scFv M3285 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFDFSTYGVSWVRQAPGKCLEWIG |
| | | YIDPVYGSTHYADSVKGRFTISRDNSKNTL |
| | | YLQMNSLRAEDTASYYCARDLYAGSSGYY |
| | | MIYSLWGQGTSVTVSS |
| SEQ ID NO: 101 | scFv M3286 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFTFSTYGVSWVRQAPGKCLEWIG |
| | | YIDPVYGSTHYADSVKGRFTISRDNSKNTL |
| | | YLQMNSLRAEDTASYYCARDLYAGSSGYY |
| | | MIYSLWGQGTSVTVSS |
| SEQ ID NO: 102 | scFv M3287 | DIQMTQSPSSLSASVGDRVTITCRSSQSVYN |
| | | NLLGWYQQKPGKAPKLLIYSASTLASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFDFSTYGVSWVRQAPGKCLEWIG |
| | | YIDPVYGSTHYADSVKGRFTISRDNSKNTL |
| | | YLQMNSLRAEDTASYYCVRDLYAGSSGYY |
| | | VIYSLWGQGTSVTVSS |
| SEQ ID NO: 103 | scFv M3293 | DIQMTQSPSSLSASVGDRVTITCRSSQSVYN |
| | | NLLGWYQQKPGKAPKLLIYSASTRASGVPS |
| | | RFSGSGSGTDFTLTISSLQPEDSATYYCQGT |
| | | YYTGDWYYPFGCGTKVEIKGGGGSGGGGS |
| | | GGGGSGGGGSQVQLVESGGGSVQPGRSLR |
| | | LSCAASGFDFSTYGVSWVRQAPGKCLEWIG |
| | | YIDPVYGSTHYADSVKGRFTISRDNSKNTL |
| | | YLQMNSLRAEDTASYYCVRDLYAGSSGYY |
| | | MIYSLWGQGTSVTVSS |

CD3 binding domain

| | | |
|---|---|---|
| SEQ ID NO: 104 | HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSC |
| SEQ ID NO: 105 | LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECS |
| SEQ ID NO: 140 | HCDR1 | STYAMN |
| SEQ ID NO: 141 | HCDR2 | RIRSKYNNYATYYADSVKG |
| SEQ ID NO: 142 | HCDR3 | HGNFGDSYVSWFAY |
| SEQ ID NO: 143 | LCDR1 | GSSTGAVTTSNYAN |
| SEQ ID NO: 144 | LCDR2 | GTNKRAP |
| SEQ ID NO: 145 | LCDR3 | ALWYSNHWV |
| SEQ ID NO: 146 | VH | EVQLVESGGGLVQPGGSLRLSCAASGFTES |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVS |
| SEQ ID NO: 147 | VL | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLG |

Bispecific antigen binding proteins
Monovalent Fab-scFv

| | | |
|---|---|---|
| SEQ ID NO: 105 | LC of M2638, | QAVVTQEPSLTVSPGGTVTLTCGSSTGAVT |
| | M2913, M3076, | TSNYANWVQQKPGKSPRGLIGGTNKRAPG |
| | M3182, M3202, | VPARFSGSLLGGKAALTISGAQPEDEADYY |
| | M3283, M3284, | CALWYSNHWVFGGGTKLTVLGQPKAAPSV |
| | M3285, M3286, | TLFPPSSEELQANKATLVCLISDFYPGAVTV |
| | M3287 and | AWKADSSPVKAGVETTTPSKQSNNKYAAS |
| | M3293 | SYLSLTPEQWKSHRSYSCQVTHEGSTVEKT |
| | | VAPTECS |
| SEQ ID NO: 106 | M2638 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSQEQL |
| | | VESGGGLVQPGGSLKLSCKASGFDFSTYGV |
| | | SWVRQAPGKGLEWIGYIDPVYGSTYYASW |
| | | VNGRFTISSHNAQNTLYLQLNSLTAADTAT |
| | | YFCVRDLYAGSSGYYMIYSLWGPGTLVTV |
| | | SSGGGGSGGGGSGGGGSGGGGSELVMTQT |
| | | PSSVSAAVGGTVTIKCQSSQSVYNNLLGWY |
| | | QQKPGQPPKLLIYSASTLASGVPSRFSGSGS |
| | | GTEFTLTISGVQADDAASYYCQGTYYNGD |
| | | WYYPFGGGTEVVVK |
| SEQ ID NO: 107 | M2913 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCQSSQSVYNNLLG |
| | | WYQQKPGKAPKLLIYSASTLASGVPSRFSG |
| | | SGSGTDFTLTISSLQPEDFATYYCQGTYYNG |
| | | DWYYPFGCGTKVEIKGGGGSGGGGSGGGG |
| | | SGGGGSEVQLVESGGGSVQPGGSLRLSCAA |
| | | SGFDFSTYGVSWVRQAPGKCLEWIGYIDPV |
| | | YGSTYYASWVNGRFTISRDNSKNTLYLQM |
| | | NSLRAEDTASYYCVRDLYAGSSGYYMIYSL |
| | | WGQGTSVTVSS |
| SEQ ID NO: 108 | M3076 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDFATYYCQGTYYNG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 109 | M3182 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDFATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 110 | M3202 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 111 | M3283 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFTFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 112 | M3284 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRESG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFTVSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 113 | M3285 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCARDLYAGSSGYYMIYSL |
| | | WGQGTSVTVSS |
| SEQ ID NO: 114 | M3286 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFTFSTYGVSWVRQAPGKCLEWIGYIDPV |
| | | YGSTHYADSVKGRFTISRDNSKNTLYLQMN |
| | | SLRAEDTASYYCARDLYAGSSGYYMIYSL |
| | | WGQGTSVTVSS |
| SEQ ID NO: 115 | M3287 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYVIYSLW |
| | | GQGTSVTVSS |
| SEQ ID NO: 116 | M3293 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTRASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |

Bivalent Fab-(scFvs)2

| | | |
|---|---|---|
| SEQ ID NO: 117 | M2873 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSQEQL |
| | | VESGGGLVQPGGSLKLSCKASGFDFSTYGV |
| | | SWVRQAPGKGLEWIGYIDPVYGSTYYASW |
| | | VNGRFTISSHNAQNTLYLQLNSLTAADTAT |
| | | YFCVRDLYAGSSGYYMIYSLWGPGTLVTV |
| | | SSGGGGSGGGGSGGGGSGGGGSELVMTQT |
| | | PSSVSAAVGGTVTIKCQSSQSVYNNLLGWY |
| | | QQKPGQPPKLLIYSASTLASGVPSRFSGSGS |
| | | GTEFTLTISGVQADDAASYYCQGTYYNGD |
| | | WYYPFGGGTEVVVK |
| SEQ ID NO: 118 | M2873 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSQEQLVESGGGLVQPGGSL |
| | | KLSCKASGFDFSTYGVSWVRQAPGKGLEW |
| | | IGYIDPVYGSTYYASWVNGRFTISSHNAQN |
| | | TLYLQLNSLTAADTATYFCVRDLYAGSSGY |
| | | YMIYSLWGPGTLVTVSSGGGGSGGGGSGG |
| | | GGSGGGGSELVMTQTPSSVSAAVGGTVTIK |
| | | CQSSQSVYNNLLGWYQQKPGQPPKLLIYSA |
| | | STLASGVPSRFSGSGSGTEFTLTISGVQADD |
| | | AASYYCQGTYYNGDWYYPFGGGTEVVVK |
| SEQ ID NO: 119 | M3134 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCQSSQSVYNNLLG |
| | | WYQQKPGKAPKLLIYSASTLASGVPSRFSG |
| | | SGSGTDFTLTISSLQPEDFATYYCQGTYYNG |
| | | DWYYPFGCGTKVEIKGGGGSGGGGSGGGG |
| | | SGGGGSEVQLVESGGGSVQPGGSLRLSCAA |
| | | SGFDFSTYGVSWVRQAPGKCLEWIGYIDPV |
| | | YGSTYYASWVNGRFTISRDNSKNTLYLQM |
| | | NSLRAEDTASYYCVRDLYAGSSGYYMIYSL |
| | | WGQGTSVTVSS |
| SEQ ID NO: 120 | M3134 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCQSSQSVYNNLLGWYQQKPGKAPKLL |
| | | IYSASTLASGVPSRFSGSGSGTDFTLTISSLQ |
| | | PEDFATYYCQGTYYNGDWYYPFGCGTKVE |
| | | IKGGGGSGGGGSGGGGSGGGGSEVQLVES |
| | | GGGSVQPGGSLRLSCAASGFDFSTYGVSWV |
| | | RQAPGKCLEWIGYIDPVYGSTYYASWVNG |
| | | RFTISRDNSKNTLYLQMNSLRAEDTASYYC |
| | | VRDLYAGSSGYYMIYSLWGQGTSVTVSS |
| SEQ ID NO: 121 | M3167 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDFATYYCQGTYYNG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 122 | M3167 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDFATYYCQGTYYNGDWYYPFGCGTKVE |
| | | IKGGGGSGGGGSGGGGSGGGGSQVQLVES |
| | | GGGSVQPGRSLRLSCAASGFDFSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYMIYSLWGQGTSVTVSS |
| SEQ ID NO: 123 | M3275 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDFATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 124 | M3275 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDFATYYCQGTYYTGDWYYPFGCGTKVE |
| | | IKGGGGSGGGGSGGGGSGGGGSQVQLVES |
| | | GGGSVQPGRSLRLSCAASGFDFSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYMIYSLWGQGTSVTVSS |
| SEQ ID NO: 125 | M3279 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 126 | M3279 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFDFSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYMIYSLWGQGTSVTVSS |
| SEQ ID NO: 127 | M3288 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFTFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 128 | M3288 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFTFSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYMIYSLWGQGTSVTVSS |
| | | EVQLVESGGGLVQPGGSLRLSCAASGFTFS |
| | | TYAMNWVRQAPGKGLEWVGRIRSKYNNY |
| SEQ ID NO: 129 | M3289 HC | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFTVSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 130 | M3289 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFTVSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYMIYSLWGQGTSVTVSS |
| SEQ ID NO: 131 | M3290 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCARDLYAGSSGYYMIYSL |
| | | WGQGTSVTVSS |
| SEQ ID NO: 132 | M3290 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFDFSTYGVSWV |
| | | RQAPGKCLEWIGYIDPVYGSTHYADSVKGR |
| | | FTISRDNSKNTLYLQMNSLRAEDTASYYCA |
| | | RDLYAGSSGYYMIYSLWGQGTSVTVSS |
| SEQ ID NO: 133 | M3291 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFTFSTYGVSWVRQAPGKCLEWIGYIDPV |
| | | YGSTHYADSVKGRFTISRDNSKNTLYLQMN |
| | | SLRAEDTASYYCARDLYAGSSGYYMIYSL |
| | | WGQGTSVTVSS |
| SEQ ID NO: 134 | M3291 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFTFSTYGVSWV |
| | | RQAPGKCLEWIGYIDPVYGSTHYADSVKGR |
| | | FTISRDNSKNTLYLQMNSLRAEDTASYYCA |
| | | RDLYAGSSGYYMIYSLWGQGTSVTVSS |
| SEQ ID NO: 135 | M3292 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTLASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYVIYSLW |
| | | GQGTSVTVSS |
| SEQ ID NO: 136 | M3292 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTLASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFDFSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYVIYSLWGQGTSVTVSS |
| SEQ ID NO: 137 | M3326 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSSLSASVGDRVTITCRSSQSVYNNLLG |
| | | WYQQKPGKAPKLLIYSASTRASGVPSRFSG |
| | | SGSGTDFTLTISSLQPEDSATYYCQGTYYTG |
| | | DWYYPFGCGTKVEIKGGGGSGGGGSGGGG |
| | | SGGGGSQVQLVESGGGSVQPGRSLRLSCAA |
| | | SGFDFSTYGVSWVRQAPGKCLEWIGYIDPV |
| | | YGSTHYADSVKGRFTISRDNSKNTLYLQMN |
| | | SLRAEDTASYYCVRDLYAGSSGYYMIYSL |
| | | WGQGTSVTVSS |
| SEQ ID NO: 138 | M3326 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSSLSASVGDR |
| | | VTITCRSSQSVYNNLLGWYQQKPGKAPKLL |
| | | IYSASTRASGVPSRFSGSGSGTDFTLTISSLQ |
| | | PEDSATYYCQGTYYTGDWYYPFGCGTKVE |
| | | IKGGGGSGGGGSGGGGSGGGGSQVQLVES |
| | | GGGSVQPGRSLRLSCAASGFDFSTYGVSWV |
| | | RQAPGKCLEWIGYIDPVYGSTHYADSVKGR |
| | | FTISRDNSKNTLYLQMNSLRAEDTASYYCV |
| | | RDLYAGSSGYYMIYSLWGQGTSVTVSS |

Target peptide

| | | |
|---|---|---|
| SEQ ID NO: 139 | PRAME target | SLLQHLIGL |
| | peptide | |

Comparator compounds

| | | |
|---|---|---|
| SEQ ID NO: 194 | Comparator 1 | GDAKTTQPNSMESNEEEPVHLPCNHSTISGT |
| | Alpha Chain | DYIHWYRQLPSQGPEYVIHGLTSNVNNRM |
| | | ASLAIAEDRKSSTLILHRATLRDAAVYYCILI |
| | | LGHSRLGNYIATFGKGTKLSVIPNIQNPDPA |
| | | VYQLRDSKSSDKSVCLFTDFDSQTNVSQSK |
| | | DSDVYITDKCVLDMRSMDFKSNSAVAWSN |
| | | KSDFACANAFNNSIIPEDT |
| SEQ ID NO: 195 | Comparator 1 | AIQMTQSPSSLSASVGDRVTITCRASQDIRN |
| | Beta Chain | YLNWYQQKPGKAPKLLIYYTSRLESGVPSR |
| | | FSGSGSGTDYTLTISSLQPEDFATYYCQQGN |
| | | TLPWTFGQGTKVEIKGGGGSGGGGSGGGG |
| | | SGGGGSGGGSEVQLVESGGGLVQPGGSLRL |
| | | SCAASGYSFTGYTMNWVRQAPGKGLEWV |
| | | ALINPYKGVSTYNQKFKDRFTISVDKSKNT |
| | | AYLQMNSLRAEDTAVYYCARSGYYGDSD |
| | | WYFDVWGQGTLVTVSSGGGGSDGGITQSP |
| | | KYLFRKEGQNVTLSCEQNLNHDAMYWYR |
| | | QDPGQGLRLIYYSQIMGDEQKGDIAEGYSV |
| | | SREKKESFPLTVTSAQKNPTAFYLCASSWW |
| | | TGGASPIRFGPGTRLTVTEDLKNVFPPEVAV |
| | | FEPSEAEISHTQKATLVCLATGFYPDHVELS |
| | | WWVNGKEVHSGVCTDPQPLKEQPALNDSR |
| | | YALSSRLRVSATFWQDPRNHFRCQVQFYG |
| | | LSENDEWTQDRAKPVTQIVSAEAWGRAD |
| SEQ ID NO: 196 | Comparator 2 | ILNVEQSPQSLHVQEGDSTKFTCSFPVKEFQ |
| | Heavy chain 1 | DLHWYRKETAKSPEFLFYFGPYGKEKKKG |
| | | RISATLNTKEGYSYLYITDSQPEDSATYLCA |
| | | LYNNYDMRFGAGTRLTVKPGGGSGGGGEV |
| | | QLVQSGAEVKKPGASVKVSCKASGYKFTS |
| | | YVMHWVRQAPGQGLEWMGYINPRNDVTK |
| | | YAEKFQGRVTLTSDTSTSTAYMELSSLRSE |
| | | DTAVYYCARGSYYDYEGFVYWGQGTLVT |
| | | VSSEPKSSDKTHTCPPCPAPPVAGPSVFLFPP |
| | | KPKDTLMISRTPEVTCVVVDVSHEDPEVKF |
| | | NWYVDGVEVHNAKTKPREEQYQSTYRVV |
| | | SVLTVLHQDWLNGKEYKCKVSNKALPASI |
| | | EKTISKAKGQPREPQVCTLPPSRDELTKNQV |
| | | SLSCAVKGFYPSDIAVEWESNGQPENNYKT |
| | | TPPVLDSDGSFFLVSKLTVDKSRWQQGNVF |
| | | SCSVMHEALHNHYTQKSLSLSP |
| SEQ ID NO: 197 | Comparator 2 | QIQMTQSPSSLSASVGDRVTITCSATSSVSY |
| | Heavy chain 2 | MHWYQQKPGKAPKRWIYDTSKLASGVPSR |
| | | FSGSGSGTDYTLTISSLQPEDAATYYCQQW |
| | | SSNPLTFGGGTKVEIKGGGSGGGGGVIQSPR |
| | | HEVTEMGQEVTLRCKPISGHNSLFWYRETP |
| | | MQGLELLIYFQNTAVIDDSGMPEDRFSAKM |
| | | PNASFSTLKIQPSEPRDSAVYFCASSPGATD |
| | | KQYFGPGTRLTVLEPKSSDKTHTCPPCPAPP |
| | | VAGPSVFLFPPKPKDTLMISRTPEVTCVVVD |
| | | VSHEDPEVKFNWYVDGVEVHNAKTKPREE |
| | | QYQSTYRVVSVLTVLHQDWLNGKEYKCK |
| | | VSNKALPASIEKTISKAKGQPREPQVYTLPP |
| | | CRDELTKNQVSLWCLVKGFYPSDIAVEWES |
| | | NGQPENNYKTTPPVLDSDGSFFLYSKLTVD |
| | | KSRWQQGNVFSCSVMHEALHNHYTQKSLS |
| | | LSP |

### Dual Peptide-MHC—Immune Cell Engaging Antigen Binding Proteins

In one aspect, the disclosure provides a bispecific antigen binding protein, comprising a first antigen binding domain comprising the antigen binding protein described above, and at last one antigen binding domain with specificity for a cell surface protein of an immune cell (e.g., CD3 on the surface of a T cell; an “immune cell binding domain”).

In certain embodiments, the immune cell is a human cell. In certain embodiments, the immune cell is selected from the group consisting of a T cell, a B cell, a natural killer (NK) cell, a natural killer T (NKT) cell, a neutrophil cell, a monocyte, and a macrophage. In certain embodiments, the immune cell is a T cell. In certain embodiments, the cell surface protein of an immune cell is selected from the group consisting of CD3, TCRα, TCRβ, the α/β T Cell Receptor, CD16 (e.g., CD16a), NKG2D, CD94/NKG2C, NKp30, NKp46, CD89, CD64, and CD32 (e.g., CD32a) on the surface of an immune cell. In certain embodiments, the cell surface protein of an immune cell is CD3.

In certain embodiments, the immune cell binding domain is an antibody, such as a CD3 targeting antibody, a CD16a targeting antibody or BMA031 or a variant thereof.

Suitable anti-CD3 binding domains are known in the art, particularly T-cell activating CD3-epsilon binding domains. The terms “CD3 binding domain” and “anti-CD3 binding domain” are used interchangeably herein. In certain embodiments of the present disclosure, the anti-CD3 binding domain is a humanized version of SP34, OKT3 or UCHT1, i.e., huSP34, huOKT3 or huUCHT1. SP34, OKT3 or UCHT1 are murine antibodies; for therapeutic applications, humanized versions i.e., huSP34, huOKT3 or huUCHT1, are preferred. In certain embodiments, the CD3 binding domain is a variant sequence of SP34, OKT3 or UCHT1 having at least about 80%, 85%, 90%, 95%, 96%, 97%, 98% or 99% identity thereto, while retaining the same specificity as its parent. In certain embodiments, the humanized variant sequence of SP34, OKT3 or UCHT1 is optimized for use in Fab format. For example, the humanized huSP34, huOKT3 or huUCHT1 may comprise 1, 2, 3, 4, 5, 6, 7, 8, 9 or more substitutions while retaining selective binding to CD3. Exemplary CD3 binding domains are disclosed in U.S. Pat. No. 6,750,325, WO2008079713, U.S. Pat. No. 7,635,475, WO2005040220, U.S. Pat. No. 7,728,114, WO9404679, U.S. Pat. No. 7,381,803, WO2008119567, WO2014110601, WO2014145806, WO2015095392, WO2016086189 and/or WO2019195535A1, each of which is incorporated herein by reference.

In certain embodiments, binding of the bispecific antigen binding protein to CD3 on a T cell, in particular on a cytotoxic T lymphocyte, triggers a cellular response of T cell. The so activated T cell may for example exhibit altered proliferation, differentiation, cytokine secretion, cytotoxic effector molecule release, cytotoxic activity, and expression of activation markers. Assays for measuring T cell activation are known to the skilled person.

Thus, in one embodiment, the antigen binding protein of the disclosure is a bispecific antigen binding protein, comprising at least a first domain targeting MHC-displayed SLLQHLIGL (SEQ ID NO: 139) and second domain targeting CD3, wherein the first domain comprises an antibody heavy chain variable (VH) domain comprising the HCDR amino acid sequences of SEQ ID NOs: 25, 26, and 27; and an antibody light chain variable (VL) domain comprising the LCDR amino acid sequences of SEQ ID NOs.: 28, 29, and 30; and the second domain targeting CD3, in particular a huSP34, huOKT3 or huUCHT1 derived antigen binding protein.

CDR, variable domain and Fab sequences of exemplary CD3 binding domains are shown in Table 2.

In certain embodiments, the CD3 binding domain comprises the HCDRs of SEQ ID NOs: 140, 141 and 142 and/or the LCDRs of SEQ ID NO.: 143, 144 and 145 (see Table 2). Thus, in certain embodiments, a bispecific antigen binding protein is provided, comprising:
(a) an at least a first domain targeting MHC-displayed SLLQHLIGL (SEQ ID NO: 139), comprising an antibody heavy chain variable (VH) domain comprising the HCDR amino acid sequences of SEQ ID NOs: 25, 26, and 27; and an antibody light chain variable (VL) domain comprising the LCDR amino acid sequences of SEQ ID NOs.: 28, 29, and 30; and
(b) a second domain targeting CD3 comprising an antibody heavy chain variable (VH) domain comprising the HCDR amino acid sequences of SEQ ID NOs: 140, 141 and 142; and an antibody light chain variable (VL) domain comprising the LCDR amino acid sequences of SEQ ID NOs.: 143, 144 and 145.

In particular embodiments, said bispecific antigen binding protein targeting MHC-displayed SLLQHLIGL (SEQ ID NO: 139) and CD3 comprises:
(a) an at least a first domain targeting MHC-displayed SLLQHLIGL (SEQ ID NO: 139), comprising an antibody heavy chain variable (VH) domain and an antibody light chain variable (VL) domain, the VH and the VL comprising:
(a1) the HCDR amino acid sequences of SEQ ID NOs: 31, 32, and 33; and the LCDR amino acid sequences of SEQ ID NOs.: 34, 35 and 36; or
(a2) the HCDR amino acid sequences of SEQ ID NOs: 37, 38 and 39; and the LCDR amino acid sequences of SEQ ID NOs.: 40, 41 and 42; or
(a3) the HCDR amino acid sequences of SEQ ID NOs: 43, 44 and 45; and the LCDR amino acid sequences of SEQ ID NOs.: 46, 47 and 48; or
(a4) the HCDR amino acid sequences of SEQ ID NOs: 49, 50 and 51; and the LCDR amino acid sequences of SEQ ID NOs.: 52, 53 and 54; or
(a3) the HCDR amino acid sequences of SEQ ID NOs: 55, 56 and 57; and the LCDR amino acid sequences of SEQ ID NOs.: 58, 59 and 60;

and (b) a second domain targeting CD3 comprising an antibody heavy chain variable (VH) domain comprising the HCDR amino acid sequences of SEQ ID NOs: 140, 141 and 142; and an antibody light chain variable (VL) domain comprising the LCDR amino acid sequences of SEQ ID NOs.: 143, 144 and 145.

In certain embodiments, the CD3 binding domain targeting comprises the VH domain of SEQ ID NO: 146 and or the VL domain of SEQ ID NO: 147, or respective variant sequences thereof having at least 90% identity thereto, such as at least 95%, 96%, 97%, 98% or 99% identity.

Said at least first antigen binding domain comprising the antigen binding protein described above, i.e., a pMHC binding domain, may e.g., be any one of a scFv, scFab, a diabody, a Fv fragment or a Fab format. In certain embodiments, the first antigen binding domain comprises an scFv and the immune cell binding domain targeting the cell surface protein of an immune cell comprises a Fab. In certain embodiments, the antigen binding protein does not comprise an Fc domain. In certain embodiments, such antigen binding protein lacking an Fc domain is a Fab-sdAb, a Fab-(sdAb)₂, a Fab-scFv or a Fab-(scFv)₂, a F(ab′)2fragment, a bis-scFv (or tandem scFv or BiTE), a DART, diabodies, a scDb, a triabody, a tetrabody, or MATCH.

In certain embodiments, the CD3 binding Fab domain light chain comprises or consists of SEQ ID NO: 105 and the Fab domain heavy chain comprises or consists of SEQ ID NO: 104, or respective variant sequences thereof having at least 90% identity thereto, such as at least 95%, 96%, 97%, 98% or 99% identity.

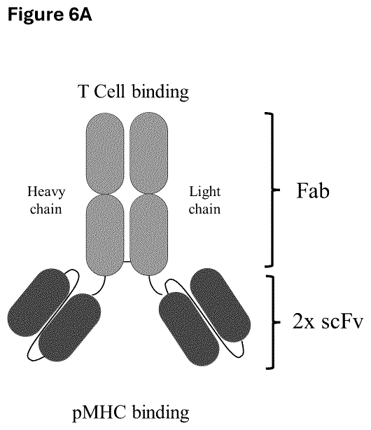

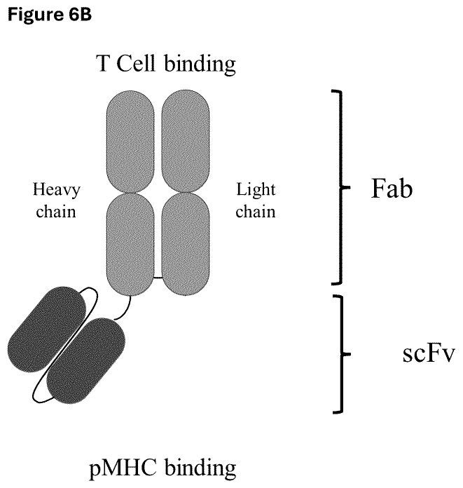

Thus, in one aspect, the disclosure provides a Fab-scFv (see e.g., FIG. 6B) comprising:
a) a Fab domain which specifically binds to a cell surface protein of an immune cell, in particular CD3, the Fab domain comprising a heavy chain (HC) and a light chain (LC);
b) a pMHC binding domain operably linked to the heavy chain, wherein the pMHC binding domain binds to a pMHC complex displaying SLLQHLIGL (SEQ ID NO: 139).

In certain embodiments, a Fab-scFv is provided, comprising (a) a CD3 targeting Fab domain comprising the HCDRs of SEQ ID NOs: 140, 141 and 142 and/or the LCDRs of SEQ ID NO.: 143, 144 and 145 and (b) a pMHC binding domain targeting MHC-displayed SLLQHLIGL (SEQ ID NO: 139), comprising an antibody heavy chain variable (VH) domain comprising the HCDR amino acid sequences of SEQ ID NOs: 25, 26, and 27; and an antibody light chain variable (VL) domain comprising the LCDR amino acid sequences of SEQ ID NOs.: 28, 29, and 30. In certain embodiments, the scFv is operably linked to the C-terminus of the CD3 targeting Fab heavy chain.

In certain embodiments, a Fab-scFv is provided, comprising (a) a CD3 targeting Fab domain comprising the HCDRs of SEQ ID NOs: 140, 141 and 142 and/or the LCDRs of SEQ ID NO.: 143, 144 and 145 and (b) a pMHC binding scFv domain targeting MHC-displayed SLLQHLIGL (SEQ ID NO: 139) which is linked to the C-terminus of the Fab heavy chain, comprising:
(1) an antibody heavy chain variable (VH) domain comprising the amino acid sequence of SEQ ID NO: 71 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 72 or a variant thereof that is at least about 75%, 78%, 79%, 80%, 81%, 83%, 85%, 90%, 95%, 96%, 97%, 98% or 99% identical thereto;
(2) an antibody heavy chain variable (VH) domain comprising the amino acid sequence of SEQ ID NO: 73 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 74 or a variant thereof that is at least 75%, 78%, 79%, 80%, 81%, 83%, 85%, 90%, 95%, 96%, 97%, 98% or 99% identical thereto;
(3) an antibody heavy chain variable (VH) domain comprising the amino acid sequence of SEQ ID NO: 75 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 76 or a variant thereof that is at least about 75%, 78%, 79%, 80%, 81%, 83%, 85%, 90%, 95%, 96%, 97%, 98% or 99% identical thereto;
(4) an antibody heavy chain variable (VH) domain comprising the amino acid sequence of SEQ ID NO: 77 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 78 or a variant thereof that is at least about 75%, 78%, 79%, 80%, 81%, 83%, 85%, 90%, 95%, 96%, 97%, 98% or 99% identical thereto;
(5) an antibody heavy chain variable (VH) domain comprising the amino acid sequence of SEQ ID NO: 79 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 80 or a variant thereof that is at least about 75%, 78%, 79%, 80%, 81%, 83%, 85%, 90%, 95%, 96%, 97%, 98% or 99% identical thereto;
(6) an antibody heavy chain variable (VH) domain comprising the amino acid sequence of SEQ ID NO: 81 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 82 or a variant thereof that is at least about 75%, 78%, 79%, 80%, 81%, 83%, 85%, 90%, 95%, 96%, 97%, 98% or 99% identical thereto;
(7) an antibody heavy chain variable (VH) domain comprising the amino acid sequence of SEQ ID NO: 83 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 84 or a variant thereof that is at least about 75%, 78%, 79%, 80%, 81%, 83%, 85%, 90%, 95%, 96%, 97%, 98% or 99% identical thereto;
(8) an antibody heavy chain variable (VH) domain comprising the amino acid sequence of SEQ ID NO: 85 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 86 or a variant thereof that is at least about 75%, 78%, 79%, 80%, 81%, 83%, 85%, 90%, 95%, 96%, 97%, 98% or 99% identical thereto;
(9) an antibody heavy chain variable (VH) domain comprising the amino acid sequence of SEQ ID NO: 87 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 88 or a variant thereof that is at least about 75%, 78%, 79%, 80%, 81%, 83%, 85%, 90%, 95%, 96%, 97%, 98% or 99% identical thereto;
(10) an antibody heavy chain variable (VH) domain comprising the amino acid sequence of SEQ ID NO: 89 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 90 or a variant thereof that is at least about 75%, 78%, 79%, 80%, 81%, 83%, 85%, 90%, 95%, 96%, 97%, 98% or 99% identical thereto; or
(11) an antibody heavy chain variable (VH) domain comprising the amino acid sequence of SEQ ID NO: 91 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 92 or a variant thereof that is at least about 75%, 78%, 79%, 80%, 81%, 83%, 85%, 90%, 95%, 96%, 97%, 98% or 99% identical thereto.

In certain embodiments, said pMHC binding scFv domain is operably linked to the C-terminus of the Fab heavy chain or the N-terminus of the Fab heavy chain. In certain embodiments, said pMHC binding scFv domain is operably linked to the C-terminus of the Fab light chain or the N-terminus of the Fab light chain.

In certain embodiments, said Fab-scFv comprises or consists of:
(1) an LC of SEQ ID NO: 105 and an HC of SEQ ID NO: 106;
(2) an LC of SEQ ID NO: 105 and an HC of SEQ ID NO: 107;
(3) an LC of SEQ ID NO: 105 and an HC of SEQ ID NO: 108;
(4) an LC of SEQ ID NO: 105 and an HC of SEQ ID NO: 109;
(5) an LC of SEQ ID NO: 105 and an HC of SEQ ID NO: 110;
(6) an LC of SEQ ID NO: 105 and an HC of SEQ ID NO: 111;
(7) an LC of SEQ ID NO: 105 and an HC of SEQ ID NO: 112;
(8) an LC of SEQ ID NO: 105 and an HC of SEQ ID NO: 113;
(9) an LC of SEQ ID NO: 105 and an HC of SEQ ID NO: 114;
(10) an LC of SEQ ID NO: 105 and an HC of SEQ ID NO: 115; or
(11) an LC of SEQ ID NO: 105 and an HC of SEQ ID NO: 116;
or respective variants of said LC and HC sequences that are at least about 75%, 78%, 79%, 80%, 81%, 83%, 85%, 90%, 95%, 96%, 97%, 98% or 99% identical thereto.

In certain embodiments, the multispecific antigen binding protein described above further comprises a second antigen binding domain, which may or may not target MHC-displayed SLLQHLIGL (SEQ ID NO: 139). In certain embodiments, the first and the second antigen binding domain are the same, so both bind to MHC-displayed SLLQHLIGL (SEQ ID NO: 139).

Thus, in one aspect, the disclosure provides a multispecific antigen binding protein comprising:
a) a Fab domain which specifically binds to a cell surface protein of an immune cell, the Fab domain comprising a heavy chain (HC) and a light chain (LC) (i.e., an immune cell binding domain);
b) a first pMHC binding domain operably linked to the heavy chain, wherein the first pMHC binding domain binds to first target peptide-MHC (pMHC) complex; and
c) a second pMHC binding domain operably linked to the light chain, wherein the second pMHC binding domain binds to a second pMHC complex,
wherein the first and/or the second pMHC binding domain is the antigen binding protein targeting MHC-displayed SLLQHLIGL (SEQ ID NO: 139) described herein.

Targeting two pMHC complexes on the surface of a target cell (e.g., a cancer cell) improves target cell engagement through avidity-enhanced binding. The enhanced binding (i.e., lower apparent K_{D} of the multivalent interaction) may in turn promote improved target cell killing relative to an antigen binding protein that only has one pMHC binding domain. The avidity-enhanced binding created by at least two pMHC binding domains may be particularly useful when targeting pMHC complexes of low copy number on the surface of a target cell (e.g., cancer cell).

As used herein, the terms “first”, “second” or “third” with respect to antigen binding domains are used for convenience of distinguishing when there is more than one antigen binding domain. Unless explicitly stated otherwise, the use of these terms is not intended to denominate a specific order or orientation.

In some embodiments, the first target pMHC complex and the second target pMHC complex are the same (i.e., they have identical sequences and the multispecific antigen binding protein is in fact bispecific). In certain embodiments, the first target pMHC complex and the second target pMHC complex are different (e.g., each complex comprises a different peptide bound to the MHC molecule or the same peptide is bound to a different HLA subtype).

In some embodiments, the first pMHC binding domain and the second pMHC binding domain are different (i.e., the binding domains bind to different epitopes). In certain embodiments, the first pMHC binding domain and the second pMHC binding domain are the same (i.e., the binding domains bind to the same epitope and/or are identical in sequence). In certain embodiments thereof, the antigen binding protein is bispecific and bivalent for the pMHC.

In some embodiments, the antigen binding protein has no more than two pMHC binding domains, i.e., is limited with regard to pMHC binding domains to one first pMHC binding domain one second pMHC binding domain, in particular when both pMHC binding domains are in scFv format and are the same. In certain embodiments, the antigen binding protein has a Fab-(scFvs)₂ scaffold, as depicted in FIG. 6A.

The Fab domains of the multispecific antigen binding proteins of the disclosure may serve as a specific heterodimerization scaffold to which the additional pMHC binding domains are linked. The natural and efficient heterodimerization properties of the heavy chain (Fd fragment) and light chain (L) of a Fab fragment makes the Fab fragment a useful scaffold. Additional binding domains may be in several different formats, including, but not limited to, another Fab domain, a scFv, or an sdAb.

In certain embodiments, the Fab domain heavy chain comprises a CH1 domain and a VH domain. In certain embodiments, the Fab domain comprises up to, or at most, 10 amino acids of an antibody hinge region, such as 5 amino acids of an antibody hinge region, at least 5 amino acids of an antibody hinge region, or 5-10 amino acids located at the C-terminus of the heavy chain of the Fab domain, and further comprises a sequence that follows the said at least 5 amino acids of an antibody hinge region and that serves as a linker connecting a first or second pMHC domain as described elsewhere herein. In certain embodiments, the Fab domain comprises the sequence stretch up to the first cysteine of the antibody hinge region. In certain embodiments, said sequence stretch is or comprises the sequence EPKSC (SEQ ID NO: 16). The presence of cysteine allows for an additional disulfide bridge which may further stabilize the antigen binding protein. In some embodiments, said at most 10 amino acids of an antibody hinge region comprises EPKSCDKTHT (SEQ ID NO: 18). The antibody hinge region may additionally comprise the sequence GGGGS (SEQ ID NO: 2) which may serve as a linker sequence to the pMHC binding domain(s). Thus, in some embodiments, a pMHC binding domain is linked to the C-terminal end of the Fab CH1 domain via any of EPKSCGGGGS (SEQ ID NO: 17), EPKSCDKTHT (SEQ ID NO: 18), EPKSCDKTHTGGGGS (SEQ ID NO: 19), DKTHT (SEQ ID NO: 20), DKTHTGGGGS (SEQ ID NO: 21) or GGGGSGGGGS (SEQ ID NO: 3) linker.

In certain embodiments, the Fab domain light chain comprises a CL domain and a VL domain. The CL domain may be followed by a linker, such as GGGGS (SEQ ID NO: 2).

Each chain of the Fab fragment can be extended at the N- or C-terminus with additional binding domains. The chains may be co-expressed in mammalian cells, where the host-cell Binding immunoglobulin protein (BiP) chaperone drives the formation of the heavy chain-light chain heterodimer (Fd:L). These heterodimers are stable, with each of the binders retaining their specific affinities. The two remaining pMHC binding domains may then be fused as scFvs or sdAbs to distinct Fab chains where each chain can be extended, e.g., at the C-terminus with an additional scFv or sdAb domain (see, for example, Schoonjans et al. J. Immunology, 165(12): 7050-7057, 2000; Schoonjans et al. Biomolecular Engineering, 17: 193-202, 2001.) An additional advantage of using Fabs as a heterodimerization unit is that Fab molecules are abundantly present in serum and therefore may be non-immunogenic when administered to a subject.

In one embodiment, the antigen binding protein of the disclosure comprises a Fab domain as well as a first and a second pMHC binding domain, both pMHC binding domains being scFvs, i.e., the molecule is a Fab-(scFv)₂. An advantage of the Fab-(scFvs)₂ scaffolds of the disclosure is the intermediate molecular size of approximately 75-110 kDa. Blinatumomab, a bispecific T cell engager (BiTE), has shown excellent results in patients with relapsed or refractory acute lymphoblastic leukemia. Because of its small size (60 kDa), blinatumomab is characterized by a short serum half-life of several hours, and therefore continuous infusion is needed (see, U.S. Pat. No. 7,112,324 B1). The antigen binding proteins of the disclosure are expected to have significantly longer half-lives in comparison to smaller bispecific antibodies, such as BiTEs like blinatumomab, and thus, do not require continuous infusion due to their favorable half-life. An intermediate sized molecule may avoid kidney clearance and provide a half-life sufficient for improved tumor accumulation. While the antigen binding proteins of the disclosure have increased plasma half-life compared to other small bispecific formats, they still retain the tumor penetration ability. On the other hand, the molecules of the instant disclosure lacking an Fc domain are expected to have a shorter half-life than larger molecules including an Fc domain. A prolonged half-life may overstimulate T cells and lead to T cell exhaustion. Also, especially in solid tumors, a large molecular weight may translate into a lower degree of tumor penetration. In some embodiments, the in vivo half-life is of about 7 days.

In certain embodiments, the antigen binding protein comprises a molecular weight of about 75 kDa to about 110 kDa (e.g., about 75 kDa, about 80 kDa, about 85 kDa, about 90 kDa, about 95 kDa, about 100 kDa, about 105 kDa or about 110 kDa). In certain embodiments, the antigen binding protein has increased serum half-life relative to an antigen binding protein with a molecular weight of less than about 60 kDa.

In certain embodiments, the first pMHC binding domain is operably linked to the C-terminus of the Fab heavy chain or the N-terminus of the Fab heavy chain. In certain embodiments, the first pMHC binding domain is operably linked to the C-terminus of the Fab light chain or the N-terminus of the Fab light chain.

In certain embodiments, the second pMHC binding domain is operably linked to the C-terminus of the Fab heavy chain or the N-terminus of the Fab heavy chain. In certain embodiments, the second pMHC binding domain is operably linked to the C-terminus of the Fab light chain or the N-terminus of the Fab light chain.

The at least first and the at least second pMHC binding domain may both be linked to either the heavy chain, or may both be linked to the light chain of the Fab domain. In some embodiments, the at least first and the at least second pMHC binding domain are not linked to the same chain of the Fab domain, i.e., one is linked to the heavy chain of the Fab domain, and the other is linked to the light chain of the Fab domain. For example, in certain embodiments, the at least first pMHC binding domain is operably linked to the C-terminus of the heavy chain of the Fab domain, and the at least second pMHC binding domain is operably linked to the C-terminus of the light chain of the Fab domain. In other embodiments, the at least first pMHC binding domain is operably linked to the C-terminus of the heavy chain of the Fab domain, and the at least second pMHC binding domain is operably linked to the N-terminus of the light chain of the Fab domain.

Suitable linker sequences between the immune cell binding domain and the pMHC binding domains include glycine polymers (Gly)ₙ; glycine-serine polymers (GlyₙSer)_{y}, wherein n and y are an integer of at least one, two, three, four, five, six, seven, or eight; glycine-alanine polymers; alanine-serine polymers; and other flexible linkers known in the art. In various embodiments, the linker sequence connecting the immune cell binding domain and the pMHC binding domain(s) is the (GGGGS)₁ (SEQ ID NO: 2) linker sequence.

In specific embodiments, the present disclosure encompasses a bispecific bivalent antigen binding protein, comprising a Fab domain which specifically binds to CD3; and no more than two pMHC binding domains, wherein both pMHC binding domains are targeting the same pMHC complex, wherein both pMHC binding domains are each a scFv, and wherein one of both pMHC binding domains is operably linked to the C-terminus of the heavy chain of the CD3 binding domain, and the other pMHC binding domain is operably linked to the C-terminus of the light chain of the CD3 binding domain. In certain embodiments, the pMHC binding domain is a scFv, more particularly each of the at least first pMHC binding domain and/or each of the at least second pMHC binding domain is a scFv. As described elsewhere herein, the pMHC binding domain may also be any one of a scFab, a diabody, or a Fab. Further, in certain embodiments, both the at least first pMHC binding domain and the at least second pMHC binding domain are each a scFv, and both the at least first pMHC binding domain and the at least second pMHC binding domain are the same. Still further, in certain embodiments thereof, the antigen binding protein is bivalent for the target pMHC complex and comprises no more than two pMHC binding domains and both said pMHC binding domains are targeting the same pMHC complex.

In certain embodiments, the immune cell binding domain, in particular the Fab domain, specifically binds to CD3 with an affinity (K_{D}) between about 1 nM to about 150 nM (e.g., 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 105 nM, 106 nM, 107 nM, 108 nM, 109 nM, 110 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 126 nM, 127 nM, 128 nM, 129 nM, 130 nM, 131 nM, 132 nM, 133 nM, 134 nM, 135 nM, 136 nM, 137 nM, 138 nM, 139 nM, 140 nM, 141 nM, 142 nM, 143 nM, 144 nM, 145 nM, 146 nM, 147 nM, 148 nM, 149 nM, 150 nM), as determined by SPR. In certain embodiments, the immune cell binding domain, in particular the Fab domain, specifically binds to CD3 with an affinity (K_{D}) between about 1 nM to about 50 nM, as determined by SPR. In certain embodiments, the immune cell binding domain, in particular the Fab domain, specifically binds to CD3 with an affinity (K_{D}) between about 1 nM to about 10 nM, e.g., about 3 nM, as determined by SPR.

In certain embodiments, the immune cell binding domain, in particular the Fab domain, specifically binds to CD3 with an affinity (K_{D}) of about 1 nM, of about 10 nM, or of about 50 nM, as determined by SPR.

In some embodiments, the association rate constant kₐ of the anti-CD3 binding domain is between about 1×10⁵ to about 1×10⁷ M⁻¹s⁻¹, such as at least 1×10⁶ M⁻¹s⁻¹ or at least 2×10⁶ M⁻¹s⁻¹.

In some embodiments, the dissociation rate constant k_{d} of the anti-CD3 binding domain is between about 1×10⁻¹ to about 1×10⁻⁶ s⁻¹, such as at least 2×10⁻³ s⁻¹, or at least 3×10⁻³ s⁻¹ or at least 4×10⁻³ s⁻¹. Without being bound to theory, a fast dissociation rate, e.g., a k_{d}-value of 2-3×10⁻³ s⁻¹, may lead to less T cell overactivation and in consequence, less cytokine release.

In one embodiment, the association rate constant kₐ and/or the dissociation rate constant k_{d} are equivalent or similar for both CD3-heterodimers CD3εγ (epsilon/gamma) and CD3εδ (epsilon/delta), i.e., there is no significant difference for either the kₐ or the k_{d} or both of the anti-CD3 binding domain to CD3εγ (epsilon/gamma) and CD3εδ (epsilon/delta) when measured under the same conditions, in particular when determined by SPR at 25° C. In certain embodiments, the association rate constant kₐ and/or the dissociation rate constant k_{d} values that are within 1-fold of each other, 1.5-fold of each other, 2-fold of each other, 2.5-fold of each other or 3-fold of each other, i.e., association rate constant kₐ values of 1×10⁵ M⁻¹s⁻¹ and 3×10⁵ M⁻¹s⁻¹.

In one embodiment, the pMHC binding domain has an affinity (K_{D}) to an MHC-displayed SLLQHLIGL (SEQ ID NO: 139) of at least about 5 nM or stronger (e.g., about 3 nM, about 2 nM, about 1.5 nM, about 1.2 nM, about 1 nM, about 0.9 nM, about 0.75 nM, about 0.5 nM, about 0.25 nM, about 0.1 nM, about 0.08 nM, about 0.06 nM, about 0.04 nM, or about 0.02 nM), for example between about 40-160 pM, such as e.g., about 80 pM.

In certain embodiments, the multispecific antigen binding protein is a Fab-(scFv)₂ and comprises (i) a single Fab domain which specifically binds to CD3 with an affinity (K_{D}) from about 1 nM to about 50 nM, (ii) a first pMHC binding scFv linked to the C-terminus of the Fab domain heavy chain and (iii) a second pMHC binding scFv linked to the C-terminus of the Fab domain light chain.

In accordance with the above, an antigen binding protein as provided by the present disclosure, in particular the at least first and/or the at least second pMHC binding domain, is highly selective.

Accordingly, in one aspect, the present disclosure provides a bispecific bivalent antigen binding protein, comprising
(i) a Fab domain which specifically binds to CD3; and
(ii) no more than two pMHC binding domains,
wherein both pMHC binding domains are targeting the same HLA-A*02:01 complex displaying SLLQHLIGL (SEQ ID NO: 139) (i.e., the antigen binding protein is bivalent with regard to the target pMHC complex), wherein both pMHC binding domains are each a scFv, and wherein one scFv is operably linked to the C-terminus of the heavy chain of the CD3 binding domain, and the other scFv is operably linked to the C-terminus of the light chain of the CD3 binding domain. Such Fab-(scFv)₂ scaffold is illustrated e.g., in FIG. 6A.

In certain embodiments, said bispecific Fab-(scFv)₂ comprises (i) a CD3 targeting Fab domain comprising the HCDRs of SEQ ID NOs: 140, 141 and 142 and/or the LCDRs of SEQ ID NO.: 143, 144 and 145 and (ii) two pMHC binding scFvs targeting MHC-displayed SLLQHLIGL (SEQ ID NO: 139), each scFv comprising an antibody heavy chain variable (VH) domain comprising the HCDR amino acid sequences of SEQ ID NOs: 25, 26, and 27; and an antibody light chain variable (VL) domain comprising the LCDR amino acid sequences of SEQ ID NOs.: 28, 29, and 30. In certain embodiments, the first scFv is operably linked to the C-terminus of the CD3 targeting Fab heavy chain and the second scFv is operably linked to the C-terminus of the CD3 targeting Fab light chain.

In certain embodiments, a Fab-(scFv)₂ is provided, comprising (a) a CD3 targeting Fab domain comprising the HCDRs of SEQ ID NOs: 140, 141 and 142 and/or the LCDRs of SEQ ID NO.: 143, 144 and 145 and (b) two pMHC binding scFvs targeting MHC-displayed SLLQHLIGL (SEQ ID NO: 139), wherein the first scFv is operably linked to the C-terminus of the CD3 targeting Fab heavy chain and the second scFv is operably linked to the C-terminus of the CD3 targeting Fab light chain, said scFvs each comprising:
(1) an antibody heavy chain variable (VH) domain comprising the amino acid sequence of SEQ ID NO: 71 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 72 or a variant thereof that is at least about 75%, 78%, 79%, 80%, 81%, 83%, 85%, 90%, 95%, 96%, 97%, 98% or 99% identical thereto;
(2) an antibody heavy chain variable (VH) domain comprising the amino acid sequence of SEQ ID NO: 73 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 74 or a variant thereof that is at least 75%, 78%, 79%, 80%, 81%, 83%, 85%, 90%, 95%, 96%, 97%, 98% or 99% identical thereto;
(3) an antibody heavy chain variable (VH) domain comprising the amino acid sequence of SEQ ID NO: 75 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 76 or a variant thereof that is at least about 75%, 78%, 79%, 80%, 81%, 83%, 85%, 90%, 95%, 96%, 97%, 98% or 99% identical thereto;
(4) an antibody heavy chain variable (VH) domain comprising the amino acid sequence of SEQ ID NO: 77 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 78 or a variant thereof that is at least about 75%, 78%, 79%, 80%, 81%, 83%, 85%, 90%, 95%, 96%, 97%, 98% or 99% identical thereto;
(5) an antibody heavy chain variable (VH) domain comprising the amino acid sequence of SEQ ID NO: 79 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 80 or a variant thereof that is at least about 75%, 78%, 79%, 80%, 81%, 83%, 85%, 90%, 95%, 96%, 97%, 98% or 99% identical thereto;
(6) an antibody heavy chain variable (VH) domain comprising the amino acid sequence of SEQ ID NO: 81 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 82 or a variant thereof that is at least about 75%, 78%, 79%, 80%, 81%, 83%, 85%, 90%, 95%, 96%, 97%, 98% or 99% identical thereto;
(7) an antibody heavy chain variable (VH) domain comprising the amino acid sequence of SEQ ID NO: 83 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 84 or a variant thereof that is at least about 75%, 78%, 79%, 80%, 81%, 83%, 85%, 90%, 95%, 96%, 97%, 98% or 99% identical thereto;
(8) an antibody heavy chain variable (VH) domain comprising the amino acid sequence of SEQ ID NO: 85 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 86 or a variant thereof that is at least about 75%, 78%, 79%, 80%, 81%, 83%, 85%, 90%, 95%, 96%, 97%, 98% or 99% identical thereto;
(9) an antibody heavy chain variable (VH) domain comprising the amino acid sequence of SEQ ID NO: 87 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 88 or a variant thereof that is at least about 75%, 78%, 79%, 80%, 81%, 83%, 85%, 90%, 95%, 96%, 97%, 98% or 99% identical thereto;
(10) an antibody heavy chain variable (VH) domain comprising the amino acid sequence of SEQ ID NO: 89 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 90 or a variant thereof that is at least about 75%, 78%, 79%, 80%, 81%, 83%, 85%, 90%, 95%, 96%, 97%, 98% or 99% identical thereto; or
(11) an antibody heavy chain variable (VH) domain comprising the amino acid sequence of SEQ ID NO: 91 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 92 or a variant thereof that is at least about 75%, 78%, 79%, 80%, 81%, 83%, 85%, 90%, 95%, 96%, 97%, 98% or 99% identical thereto.

In certain embodiments of such Fab-(scFv)₂, the CD3 targeting Fab domain of (a) comprises the VH domain of SEQ ID NO: 146 and or the VL domain of SEQ ID NO: 147, or respective variant sequences thereof having at least 90% identity thereto, such as at least 95%, 96%, 97%, 98% or 99% identity.

In certain embodiments of such Fab-(scFv)₂, the CD3 targeting Fab domain of (a) comprises or consists of SEQ ID NO: 105 and the Fab domain heavy chain comprises or consists of SEQ ID NO: 104, or respective variant sequences thereof having at least 90% identity thereto, such as at least 95%, 96%, 97%, 98% or 99% identity.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 117; and a LC comprising or consisting of an amino acid sequence that is at least about 90%, 95%, 96%, 97%, 98%, 99% or 100% identical to the amino acid sequence of SEQ ID NO: 118.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 119; and a LC comprising or consisting of an amino acid sequence that is at least about 90%, 95%, 96%, 97%, 98%, 99% or 100% identical to the amino acid sequence of SEQ ID NO: 120.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 121; and a LC comprising or consisting of an amino acid sequence that is at least about 90%, 95%, 96%, 97%, 98%, 99% or 100% identical to the amino acid sequence of SEQ ID NO: 122.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 123; and a LC comprising or consisting of an amino acid sequence that is at least about 90%, 95%, 96%, 97%, 98%, 99% or 100% identical to the amino acid sequence of SEQ ID NO: 124.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 125; and a LC comprising or consisting of an amino acid sequence that is at least about 90%, 95%, 96%, 97%, 98%, 99% or 100% identical to the amino acid sequence of SEQ ID NO: 126.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 127; and a LC comprising or consisting of an amino acid sequence that is at least about 90%, 95%, 96%, 97%, 98%, 99% or 100% identical to the amino acid sequence of SEQ ID NO: 128.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 129; and a LC comprising or consisting of an amino acid sequence that is at least about 90%, 95%, 96%, 97%, 98%, 99% or 100% identical to the amino acid sequence of SEQ ID NO: 130.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 131; and a LC comprising or consisting of an amino acid sequence that is at least about 90%, 95%, 96%, 97%, 98%, 99% or 100% identical to the amino acid sequence of SEQ ID NO: 132.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 133; and a LC comprising or consisting of an amino acid sequence that is at least about 90%, 95%, 96%, 97%, 98%, 99% or 100% identical to the amino acid sequence of SEQ ID NO: 134.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 135; and a LC comprising or consisting of an amino acid sequence that is at least about 90%, 95%, 96%, 97%, 98%, 99% or 100% identical to the amino acid sequence of SEQ ID NO: 136.

In one embodiment, such bispecific Fab-(scFv)₂ comprises a HC comprising or consisting of an amino acid sequence that is at least about 90%, 95%, 96%, 97%, 98%, 99% or 100% identical to the amino acid sequence of SEQ ID NO: 137; and a LC comprising or consisting of an amino acid sequence that is at least about 90%, 95%, 96%, 97%, 98%, 99% or 100% identical to the amino acid sequence of SEQ ID NO: 138.

In certain embodiments, a bispecific antigen binding molecule of the disclosure, in particular a bispecific and bivalent Fab-(scFv)₂, shows high potency on target-positive cancer cells with varying target expression levels. In one embodiment, said antigen binding protein shows potent in vitro cell killing activity, e.g., with an EC50 of about 12 pM with OV56 cells.

As shown in the Examples, the bispecific antigen binding molecules described herein show specific cancer cell killing of PRAME-positive cancer cells and no killing of PRAME-negative cancer cells. In certain embodiments, PRAME-positive cancer cell is a HLA-A*02:01-positive PRAME-positive cancer cell. In certain embodiments, said HLA-A*02:01-positive PRAME-positive cancer cell is a NCI-H1703, NCI-H1755, OV56, SW982, SK-MEL-5, COV318 and/or EFO-27 cancer cell. In certain embodiments, PRAME-negative cancer cell is a HLA-A*02:01-positive PRAME-negative cancer cell. In certain embodiments, said HLA-A*02:01-positive PRAME-negative cancer cell is a TYK-nu, DBTRG-05MG, SK-MEL-30, SW620, CFPAC-1 and/or Colo678 cancer cell.

In certain embodiments, the antigen binding protein of the disclosure, in particular the bispecific antigen binding protein such as the Fab-(scFv)₂, exhibits a broad safety profile on PRAME-negative healthy primary cells. In certain embodiments, said safety profile is broader than the one of a competitor molecule. In certain embodiments, the bispecific antigen binding protein shows little to no binding to an off-target peptide selected from Table 6, in particular SEQ ID NOs.: 190, 192, 198, 199, 201, 202 and/or 203. In certain embodiments, the K_{D} value for off-target peptide binding is at least about 20-fold higher, e.g. at least about 30-fold, 50 fold, 75-fold, 100 fold, 125-fold, 150-fold, 175-fold, 200-fold, 225-fold, 250-fold, 275-fold, 300-fold, 325-fold, 350-fold or 325 fold higher than the K_{D} value for the target pMHC displaying SEQ ID NO.: 139 (i.e. lower affinity for the off-target peptide than for the target peptide). In other embodiments, no binding to the off-target peptide can be detected.

In certain embodiments, the antigen binding protein of the disclosure, in particular the bispecific antigen binding protein such as the Fab-(scFv)₂, show no reactivity to primary human cells of various origins spanning essential human tissues expressing HLA-A*02:01. In certain embodiments, said primary human cells are from the heart, such as cardiomyocytes (HCM_718; HCM_693; HCM_746), cardiac microvascular endothelial cells (HCMEC_693; HCMEC_798; HCMEC_147) and/or cardiac fibroblasts (HCF_243; HCF_251). In certain embodiments, said primary human cells are from the aorta, such as aortic endothelial cells (HAEC_22171) and/or aortic smooth muscle cells (HAoSMC_735). In certain embodiments, said primary human cells are from the lung, such as pulmonary microvascular endothelial cells (HPMEC_760; HPMEC_197), small airway epithelial cells (SAEC_33436), pulmonary fibroblasts (HPF_197; NHLF_76039), tracheal smooth muscle cells (HTSMC_084) and/or pulmonary artery smooth muscle cells (PASMC_01036). In certain embodiments, said primary human cells are from the brain, such as astrocytes (NHA_72445). In certain embodiments, said primary human cells are from the liver, such as hepatocytes (Human Hepatocytes_11621). In certain embodiments, said primary human cells are from the muscle, such as skeletal muscle cells (SkMC_683). In certain embodiments, said primary human cells are from the blood, such as peripheral blood mononuclear cells (e.g., PBMCs which can be isolated from one or more patient donors, see Examples). In certain embodiments, said primary human cells are from the kidney, such as renal proximal tubule epithelial cells (e.g., RPTEC_82573, RPTEC_49985 and/or RPTEC_95730) and/or renal cortical epithelial cells (HRCE_49986).

In certain embodiments, the antigen binding protein of the disclosure demonstrates no cross-reactivity to normal human primary cells. Such cross-reactivity may be determined by co-culturing HLA-A*02:01+ normal human primary cells with human PBMCs and the antigen binding protein and quantifying Granzyme B release after 24 h, e.g. by ELISA. See Example 8 for an exemplary set-up.

In certain embodiments, the antigen binding protein of the disclosure, in particular the bispecific antigen binding protein such as the Fab-(scFv)₂, shows a broader window between on-target/on-tumor and on-target/off-tumor reactivity than a competitor molecule. Such window may e.g., be measured in co-cultures of PBMCs with OV56 cells or with HLA-A*02:01+ normal human primary cells of renal origin known to express low levels of PRAME and quantifying Granzyme B release after 24 h. See Example 8 for an exemplary set-up.

In certain embodiments, the antigen binding protein of the disclosure, in particular the bispecific antigen binding protein such as the Fab-(scFv)₂, have favorable biophysical characteristics and manufacturability properties. For example, stability and solubility parameters are important for immunotherapeutical drugs. In certain embodiments, the antigen binding protein of the disclosure remains monomeric for prolonged periods of time and at high concentrations. In certain embodiments, the antigen binding protein of the disclosure, in particular the bispecific antigen binding protein can be concentrated to 15 mg/ml, 16 mg/ml, 17 mg/ml, 18 mg/ml, 19 mg/ml or up to 20 mg/ml. In certain embodiments, the antigen binding protein of the disclosure, in particular the bispecific antigen binding protein such as a Fab-(scFv)₂, is stable under simulated physiological conditions. In certain embodiments, and as shown in the Examples (see also J. Schuster et al, Journal of Pharmaceutical Science, 110 (2021), 2386-2394 for more detail), after incubation in carbonate-based buffer at 20 mg/ml for 7 days at simulated s.c. physiological conditions (34° C., pH 7.4), the monomer content of said bispecific antigen binding protein is at least 94%, 95%, 96%, 97%, 98%, 99% or 100% of the initial monomeric content, as determined by SEC-HPLC.

In certain embodiments, the bispecific antigen binding protein of the disclosure, in particular the Fab-(scFv)₂, shows a lower Interferon 7 cytokine release than a comparator molecule which may translate into a decreased risk of causing a potential cytokine release syndrome upon administration to a patient or in an in vivo model. Cytokine release elicited by a bispecific antigen binding protein may e.g. be determined by measuring Interferon 7 release of HLA-A*02:01-positive PRAME-positive cancer cells co-incubated for 24 h with PBMCs and said bispecific antigen binding protein. Interferon 7 release may e.g. be determined by ELISA.

### Variants and Competitor Proteins

As contemplated throughout the description, also encompassed are variants of the sequences disclosed herein. A variant amino acid or nucleic acid sequence differs from its parental sequence by virtue of insertion (including addition), deletion and/or substitution of one or more amino acid residues or nucleobases, respectively, while retaining at least one desired property of the parent sequence disclosed herein, e.g., specific antigen binding, efficacy on target positive tumor cells, stability (e.g., serum stability, thermal stability, and/or storage stability), producibility (e.g., expression levels), safety (e.g., reactivity in healthy tissues, Granzyme B release, proinflammatory cytokine IFN gamma antigen-positive or antigen-negative cell lines, and/or low to no induction of pro-inflammatory cytokines IL-2, IL-6 and TNF alpha cytokine release), efficacy (e.g., tumor growth inhibition, tumor eradication, and/or T cell activating properties as e.g. determined in vitro), or binds MHC-displayed SLLQHLIGL (SEQ ID NO: 139) in a similar affinity range.

Importantly, the variant retains specific binding to the target. In certain embodiments, the variant antigen binding protein retains binding to the target pMHC complex of at least 50%, such as 60%, 70%, 80%, 90% or 95% of the equilibrium dissociation constant K_{D} of the reference antigen binding protein (i.e., the corresponding antigen binding protein without said substitutions, insertions, and/or deletions) when measured under identical conditions. Variants may be artificially engineered or naturally occurring, such as e.g., allelic or splice variants. In some embodiments, the variant antigen binding protein comprises an amino acid sequence being at least about 80%, 85%, 90%, 95%, 96%, 97%, 98% or 99% identical to an amino acid sequence disclosed herein, while retaining specific antigen binding. In certain embodiments, said variant antigen sequence comprises: the HCDR1 amino acid sequence of SEQ ID NO: 25, the HCDR2 amino acid sequence of SEQ ID NO: 26, the HCDR3 amino acid sequence of SEQ ID NO: 27, the LCDR1 amino acid sequence of SEQ ID NO: 28, the LCDR2 amino acid sequence of SEQ ID NO: 29, and/or the LCDR3 amino acid sequence of SEQ ID NO: 30, in particular
(i) the HCDR amino acid sequences of SEQ ID NOs: 31, 32, and 33; and the LCDR amino acid sequences of SEQ ID NOs.: 34, 35 and 36; or
(ii) the HCDR amino acid sequences of SEQ ID NOs: 37, 38 and 39; and the LCDR amino acid sequences of SEQ ID NOs.: 40, 41 and 42; or
(iii) the HCDR amino acid sequences of SEQ ID NOs: 43, 44 and 45; and the LCDR amino acid sequences of SEQ ID NOs.: 46, 47 and 48; or
(iv) the HCDR amino acid sequences of SEQ ID NOs: 49, 50 and 51; and the LCDR amino acid sequences of SEQ ID NOs.: 52, 53 and 54; or
(v) the HCDR amino acid sequences of SEQ ID NOs: 55, 56 and 57; and the LCDR amino acid sequences of SEQ ID NOs.: 58, 59 and 60.

In certain embodiments, a variant antigen binding protein retains specific binding to the target pMHC and/or CD3, respectively, and/or competes with the antigen binding protein disclosed herein for binding to its target.

In certain embodiments, the antigen binding proteins disclosed herein have at least one desired property over a competitor protein against the same target (i.e., MHC-displayed SLLQHLIGL (SEQ ID NO: 139)). The term “competitor” as used herein refers to any reference molecule which may be used to test or compare the properties and/or efficacy of the antigen binding protein according to the present invention. The term “competitor” as used herein refers for example to another antigen binding protein, such as a mono- and/or multispecific antigen binding protein, in particular targeting the same target protein(s) (i.e., MHC-displayed SLLQHLIGL (SEQ ID NO: 139) and/or CD3).

The at least one desired property may e.g. be selected from specific antigen binding, efficacy on target positive tumor cells, stability (e.g., serum stability, thermal stability, and/or storage stability), producibility (e.g., expression levels), safety (e.g., reactivity in healthy tissues, Granzyme B release, proinflammatory cytokine IFN gamma antigen-positive or antigen-negative cell lines, and/or low to no induction of pro-inflammatory cytokines IL-2, IL-6 and TNF alpha cytokine release), efficacy (e.g., tumor growth inhibition, tumor eradication, and/or T cell activating properties as e.g. determined in vitro), or binds MHC-displayed SLLQHLIGL (SEQ ID NO: 139) with better affinity. In certain embodiments, the antigen binding protein of the disclosure is more specific than a competitor protein for MHC-displayed SLLQHLIGL (SEQ ID NO: 139) and shows a better off-target profile. In certain embodiments, the antigen binding protein shows less or no binding to one or more pMHCs displaying an off-target peptide sequences selected from SEQ ID NOs.: 186-203, compared to the competitor molecule. In certain embodiments, the antigen binding protein of the disclosure is more potent than a competitor protein. In certain embodiments, the antigen binding protein of the disclosure is less toxic than a competitor protein.

In certain embodiments, the antigen binding protein of the disclosure shows a broader therapeutic window towards normal primary renal cells over a competitor protein. As used herein, the term “therapeutic window” refers to the dosing parameter, such as dose range, of the antigen binding protein or a composition comprising said antigen binding protein, in which the desired benefit for treatment is provided and undesirable or adverse effects, such as toxicity, are limited or not existing. For example, “therapeutic window” refers to the safety of the antigen binding protein according to the present invention or a composition comprising said antigen binding protein. For example, the “therapeutic window” of the antigen binding protein according to the present invention is determined by assaying the target cell killing effect (cytotoxicity) of the antigen binding protein according to the present in relation to the release of cytokines, such as IFNγ (IFN-gamma). For example, an improved therapeutic window is characterized by a high target cell killing effect (cytotoxicity) and moderate/reduced IFNγ release. In one embodiment, the antigen binding protein according to the present invention or the composition comprising such antigen binding protein, respectively, has an improved therapeutic window compared to competitors, characterized by a similar target cell killing effect (cytotoxicity), but a reduced IFNγ release. In one embodiment, the antigen binding protein according to the present invention or the composition comprising such antigen binding protein, respectively, has an improved therapeutic window compared to competitors, characterized by an improved target cell killing effect (cytotoxicity) at similar IFNγ release.

In certain embodiments, the antigen binding protein of the disclosure shows a larger therapeutic index (TI) as measured on normal primary renal cells over a competitor protein. In certain embodiments, the therapeutic index is more than 100-fold to the most reactive donor. As used herein, the term “therapeutic index” refers to a range of doses at which the antigen binding protein or a composition comprising it, is effective without unacceptable adverse events.

### Chimeric Antigen Receptors

In one aspect, the disclosure provides chimeric antigen receptors (CARs) and immune cells engineered to express such CARs, comprising the antigen binding proteins described herein. As used herein, the term “chimeric antigen receptor” or “CAR” refers to a receptor that is capable of activating an immune cell in response to antigen binding. CARs are recombinant membrane spanning molecules and are advantageously expressed on immune cells. Their structure typically comprises (i) an extracellular domain (ectodomain or antibody domain), (ii) a transmembrane domain and (iii) a cytoplasmic domain (endodomain or intracellular signaling domain).

The ectodomain (i.e., antibody domain) typically comprises a scFv but other antigen binding protein formats may also be used. A spacer connects the ectodomain and the transmembrane domain, which in turn is connected to an endodomain. Upon binding of the ectodomain to the antigen, the receptors cluster and an activation signal is transmitted to the cell which results in initiation of an immune response. First generation CARs have a simply structured endodomain comprising CD3-zeta. To increase the activation signal, a co-stimulatory domain was added in the second-generation CARs; and third generation CARs include two or more co-stimulatory domains (Maus M V et al (2014) Blood, 123: 2625-2635). Said co-stimulatory domains may be selected from the group consisting of CD28, OX40 and/or 4-1BB. Apart from CD3-zeta, other ITAM-containing domains have been explored including the Fc receptor for IgE-γ domain.

Suitable immune cells include, without being limited to, T cells, Natural Killer T (NKT) cells, natural killer (NK) cells, human embryonic stem cells, hematopoietic stem cells (HSC) or induced pluripotent stem cells (iPS). Such T cell may be a cytotoxic T lymphocyte (CTL), a regulatory T lymphocyte, an inflammatory T-lymphocytes, or a helper T-lymphocyte or a gamma-delta T cell. The T cell may be a CD4+ or CD8+ or a mixed population of CD4+ and CD8+ cells.

In one aspect, the disclosure provides a chimeric antigen receptor (CAR) that specifically recognizes a peptide-MHC, comprising: i) an antigen binding protein with specificity to the peptide-MHC; ii) a transmembrane domain; and iii) an intracellular signaling domain, wherein the peptide-MHC is MHC-displayed SLLQHLIGL (SEQ ID NO: 139).

In certain embodiments, the CAR comprises a scFv comprising or consisting of the amino acid sequence of any of SEQ ID NOs: 93-103, or a variant as described herein. In certain embodiments, said variant is at least about 90%, 95%, 96%, 97%, 98% or 99% identical to the amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, or SEQ ID NO: 103, respectively. In certain embodiments thereof, the variant comprises the HCDR1 amino acid sequence of SEQ ID NO: 25, the HCDR2 amino acid sequence of SEQ ID NO: 26, the HCDR3 amino acid sequence of SEQ ID NO: 27, the LCDR1 amino acid sequence of SEQ ID NO: 28, the LCDR2 amino acid sequence of SEQ ID NO: 29, and/or the LCDR3 amino acid sequence of SEQ ID NO: 30.

In certain embodiments, the transmembrane domain is selected from the group consisting of an artificial hydrophobic sequence and transmembrane domains of a type I transmembrane protein, an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the intracellular signaling domain is selected from the group consisting of cytoplasmic signaling domains of a human CD3 zeta chain, FcγRIII, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

The antibody domain may be any of the antigen binding proteins outlined above. Thus, in certain embodiments, the antibody domain comprises an antibody variable light domain (VL) comprising an amino acid sequence represented by the formula LFR1-CDRL1-LFR2-CDRL2-LFR3-CDRL3-LFR4. In certain embodiments, the antibody domain comprises an antibody variable heavy domain (VH) comprising an amino acid sequence represented by the formula HFR1-CDRH1-HFR2-CDRH2-HFR3-CDRH3-HFR4. In certain embodiments, the antibody domain comprises a scFv as described elsewhere herein. In certain embodiments, the antigen binding protein retains stability and remains at least 94%, 95%, 96%, 97%, 98%, 99% or 100% monomeric after incubation for 14 days at 4° C. in PBS at 1 mg/mL and/or 10 mg/mL as determined by SEC-HPLC.

### Reduction of Anti-Drug Antibody Binding

Anti-drug antibodies (ADAs) may affect the risk profile and efficacy of a biological drug. If neutralizing, they may block the drug's ability to bind to its target. It is therefore a regulatory requirement to test biologic drugs for the binding of anti-drug antibodies and their neutralizing potential. Anti-drug antibody assays are e.g., detailed in WO2007101661A1 (Hoffmann La Roche), WO2018178307A1 (Ablynx), WO2021046316A2 (Adverum Biotechnologies, Charles River), and US20180088140A1 (Genzyme Corporation), each of which is incorporated herein by reference.

Anti-drug antibodies binding to a tumor targeting domain of an antigen binding protein may lead to clustering of said antigen binding protein when each variable domain of the ADA binds to one tumor targeting domain of two antigen binding proteins. The two or more CD3 binding domains on said antigen binding protein cluster and overstimulate the targeted T cell in the absence of target engagement, thereby leading to off-target toxicity. Unspecific stimulation of the T-cells may lead to systemic cytokine release.

Generally, there is a need in the art to develop safer and more effective bispecific antibodies for cancer immunotherapy.

The inventors have found that certain mutations in the tumor antigen binding domain of a T cell engager reduce ADA response and at the same time reduce nonspecific T cell stimulation in the absence of target engagement. Thereby, a highly effective and safe approach for cancer immunotherapy is provided.

For such purpose, a variable heavy chain amino acid at position 11, 89, and/or 108, according to Kabat numbering, is substituted with a polar amino acid; and/or serine (S) at position 113 is deleted, according to Kabat numbering. Such substitution is particularly favorable when the binding domain is in scFv format. In case of a Fab(scFv)₂, one or both scFvs may comprise such substitution or deletion.

In certain embodiments, the polar amino acid is serine (S) and/or threonine (T).

In certain embodiments, the heavy chain amino acid is substituted with serine (S) at heavy chain amino acid position 11, serine (S) or threonine (T) at heavy chain amino acid position 89, and/or serine (S) or threonine (T) at heavy chain amino acid position 108, according to Kabat numbering.

In certain embodiments, the heavy chain amino acid is substituted with serine (S) at heavy chain amino acid position 11, serine (S) at heavy chain amino acid position 89, and serine (S) at heavy chain amino acid position 108, according to Kabat numbering.

### Expression of Antigen Binding Proteins

In one aspect, polynucleotides or nucleic acids encoding the antigen binding proteins (including the multispecific antigen binding proteins) disclosed herein are provided, such as isolated polynucleotides or nucleic acids which are typically synthetic. Methods of making an antigen binding protein expressing these polynucleotides or nucleic acids are also provided.

Polynucleotides encoding the antigen binding proteins disclosed herein are typically inserted in a cloning vector or into an expression vector for introduction into host cells that may be used to produce the desired quantity of the antigen binding proteins. Accordingly, in certain aspects, the invention provides expression vectors comprising the polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. (e.g., polynucleotides or nucleic acids encoding the antigen binding proteins disclosed herein) in a cell. A vector may be a self-replicating nucleic acid structure or incorporate into the genome upon introduction into the host cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the antigen binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antigen binding protein such as an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature. In one embodiment, the host cell is not a cell within a human body.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the antigen binding proteins featured in the invention can also be expressed in non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

In certain embodiments, signal peptides may be encompassed in the reproduced sequences. In such case, the sequences shall be deemed disclosed with and without signal peptides. A readily available tool to identify signal peptides in a given protein sequence is SignalP—6.0 provided by Dansk Technical University under https://lnkd.in/e8mWfWv3.

In another aspect, the disclosure provides a nucleic acid encoding the antigen binding proteins recited above, such as the bispecific antigen binding protein recited above.

In another aspect, the disclosure provides an expression vector comprising the nucleic acid recited above.

In another aspect, the disclosure provides a host cell comprising the expression vector recited above.

In another aspect, the disclosure provides a method of manufacturing the antigen binding proteins recited above, such as the bispecific antigen binding protein recited above, comprising the steps of:
(i) cultivating the host cell recited above under conditions allowing expression of the antigen binding proteins;
(ii) recovering the antigen binding proteins; and optionally
(iii) further purifying and/or modifying and/or formulating the antigen binding proteins.

In certain embodiments, a method of manufacturing a bispecific antigen binding protein es described above is provided, comprising the steps of:
(i) cultivating the host cell recited above under conditions allowing expression of the bispecific antigen binding protein;
(ii) recovering the bispecific antigen binding protein; and optionally
(iii) further purifying and/or modifying and/or formulating the bispecific antigen binding protein.

In certain embodiments, a method of manufacturing a CAR-expressing cell as described above is provided, comprising the steps of:
(i) introducing a nucleic acid encoding the CAR into the cell to produce the CAR-expressing cell; and optionally
(ii) isolating the CAR-expressing cell.

### Engineering and Optimization of Antigen Binding Proteins

The antigen binding proteins, including the multispecific antigen binding proteins, of the disclosure may be engineered or optimized. As used herein, “optimized” or “optimization” refers to the alteration of an antigen binding protein to improve one or more functional properties. Alteration includes, but is not limited to, deletions, substitutions, additions, and/or modifications of one or more amino acids within an antigen binding protein.

As used herein, the term “functional property” is a property of an antigen binding protein for which an improvement (e.g., relative to a conventional antigen binding protein, such as an antibody) is desirable and/or advantageous to one of skill in the art, e.g., in order to improve the manufacturing properties or therapeutic efficacy of an antigen binding protein. In one embodiment, the functional property is stability (e.g., thermal stability). In another embodiment, the functional property is solubility (e.g., under cellular conditions). In yet another embodiment, the functional property is aggregation behavior. In still another embodiment, the functional property is protein expression (e.g., in a prokaryotic cell). In yet another embodiment the functional property is refolding behavior following inclusion body solubilization in a manufacturing process. In certain embodiments, the functional property is not an improvement in antigen affinity. In another embodiment, the improvement of one or more functional properties has no substantial effect on the affinity of the antigen binding protein.

Alterations, such as deletions, substitutions, and/or insertions, can be introduced into parental sequences by a variety of standard techniques known in the art, such as combinatorial chemistry, site-directed DNA mutagenesis, PCR-mediated and/or cassette mutagenesis, peptide/protein chemical synthesis, chemical reaction specifically modifying reactive groups in the parental binding member. The variants so formed can be tested by routine methods for their chemical, biological, biophysical and/or biochemical properties, e.g., by the methods described elsewhere herein.

In certain embodiments, the substitution is a conservative amino acid substitution. As used herein, the term “conservative substitution” refers to replacing an amino acid with a replacement amino acid that is physically, biologically, chemically and/or functionally similar to the replacement amino acid, e.g., has a similar size, shape, electric charge and/or chemical properties, including the ability to form covalent or hydrogen bonds. Non-conservative substitutions, in contrast, may lead to substantial changes, e.g., with respect to the charge, dipole moment, size, hydrophilicity, hydrophobicity or conformation of the antigen binding protein.

In certain embodiments, the antigen binding protein of the disclosure is an scFv and is optimized by identifying preferred amino acid residues to be substituted, deleted, and/or added at amino acid positions of interest (e.g., amino acid positions identified by comparing a database of scFv sequences having at least one desirable property, e.g., as selected with Quality Control (QC) assay, versus a database of mature antibody sequences, e.g., the Kabat database) in an antigen binding protein. Thus, the disclosure further provides “enrichment/exclusion” methods for selecting a particular amino acid residue. Still further, the disclosure provides methods of engineering antigen binding proteins (e.g., scFvs) by mutating particular framework amino acid positions identified using the “functional consensus” approach described herein. In certain embodiments, the framework amino acid positions are mutated by substituting the existing amino acid residue by a residue which is found to be an “enriched” residue using the “enrichment/exclusion” analysis methods described herein. In one aspect, the disclosure provides a method of identifying an amino acid position for mutation in a single chain antibody (scFv), the scFv having VH and VL amino acid sequences, the method comprising: a) entering the scFv VH, VL or VH and VL amino acid sequences into a database that comprises a multiplicity of antibody VH, VL or VH and VL amino acid sequences such that the scFv VH, VL or VH and VL amino acid sequences are aligned with the antibody VH, VL or VH and VL amino acid sequences of the database; b) comparing an amino acid position within the scFv VH or VL amino acid sequence with a corresponding position within the antibody VH or VL amino acid sequences of the database; c) determining whether the amino acid position within the scFv VH or VL amino acid sequence is occupied by an amino acid residue that is conserved at the corresponding position within the antibody VH or VL amino acid sequences of the database; and d) identifying the amino acid position within the scFv VH or VL amino acid sequence as an amino acid position for mutation when the amino acid position is occupied by an amino acid residue that is not conserved at the corresponding position within the antibody VH or VL amino acid sequences of the database. ScFv optimization is described in further detail in WO2008110348, WO2009000099, WO2009000098, and WO2009155725, all of which are incorporated herein by reference.

In those aspects of the disclosure where the presence of an Fc domain is practicable, the antigen binding protein may comprise an Fc domain which is modified such that it does not induce cytotoxic immune responses and/or does not activate complement. For example, one or more substitutions may be introduced into the Fc domain so that its ADCC/ADCP or CDC effector function is inactivated. Such antigen binding protein has the advantage of increased half-life when compared to antibody fragments with a molecular weight below 60 kDa, without mediating cytotoxic immune responses.

Certain mutations in the antigen binding domains of a T cell engager were found to reduce ADA response and at the same time reduce nonspecific T cell stimulation in the absence of target engagement. Thus, the antigen binding protein of the disclosure, in particular when in the scFv format, may comprise a variable heavy chain having a non-polar amino acid at position 11, 89 and/or 108, according to Kabat numbering. For example, the variable heavy chain comprises: leucine (L) or serine (S) at amino acid position 11, according to Kabat numbering; valine (V), serine (S), or threonine (T) at amino acid position 89, according to Kabat numbering; and/or leucine (L), serine (S), or threonine (T) amino acid position 108, according to Kabat numbering.

Chemical and/or biological modifications

In one aspect, the antigen binding protein (such as the multispecific antigen binding protein described above) is chemically and/or biologically modified. For example, the antigen binding protein may be glycosylated, phosphorylated, hydroxylated, PEGylated, HESylated, PASylated, XTENylated, sulfated, labeled with dyes and/or radioisotopes, conjugated with enzymes and/or toxins, and/or Albumin binding or fusion technology. Likewise, any nucleic acid sequence, plasmid or vector and/or host cell described herein may be modified accordingly.

Such modification may for example be done to optimize pharmacokinetics, the water solubility or to lower side effects. For example, PEGylation, PASylation, XTENylation, HESylation and/or the fusion to serum albumin may be applied to slow down renal clearance, thereby increasing plasma half-life time of the antigen binding protein. In ne embodiment, the antigen binding molecules of the disclosure are operably linked to human serum albumin. In one embodiment, a modification adds a different functionality to the antigen binding protein, for example, a detection label for diagnostics or a toxin to combat cancer cells even more efficiently.

Alternatively, or additionally, in some embodiments, the antigen binding proteins and other polypeptides provided herein undergo co- and post-translational modifications as known in the art. Examples of post-translational modifications include, but are not limited to, disulfide bond formation, glycosylation, cyclization (such as e.g., N-terminal pyroglutamate formation), have a N-terminal or C-terminal residue removed or “clipped” (for example, C-terminal lysine residues are often removed during the manufacturing process), deamidation, isomerization, oxidation, glycation, acylation, fucosylation, peptide bond cleavage, non-reductible cross-linking, truncation, and/or have part or all of a signal sequence incompletely processed.

In certain embodiments, the CD3 antigen binding domain comprises an N-terminal truncation of 1 or more amino acids (e.g., a N-terminal truncation of 1, 2, 3, 4, or 5 amino acids). In certain embodiments, the CD3 antigen binding domain comprises a C-terminal truncation of 1 or more amino acids (e.g., a C-terminal truncation of 1, 2, 3, 4, or 5 amino acids). In certain embodiments, the N-terminal and/or C-terminal truncation is a truncation of the Fab-(scFv)₂ of a heavy chain amino acid sequence of SEQ ID NO: 117 and a light chain amino acid sequence of SEQ ID NO: 118. In certain embodiments, the N-terminal and/or C-terminal truncation is a truncation of the Fab-(scFv)₂ of a heavy chain amino acid sequence of SEQ ID NO: 119 and a light chain amino acid sequence of SEQ ID NO: 120. In certain embodiments, the N-terminal and/or C-terminal truncation is a truncation of the Fab-(scFv)₂ of a heavy chain amino acid sequence of SEQ ID NO: 121 and a light chain amino acid sequence of SEQ ID NO: 122. In certain embodiments, the N-terminal and/or C-terminal truncation is a truncation of the Fab-(scFv)₂ of a heavy chain amino acid sequence of SEQ ID NO: 123 and a light chain amino acid sequence of SEQ ID NO: 124. In certain embodiments, the N-terminal and/or C-terminal truncation is a truncation of the Fab-(scFv)₂ of a heavy chain amino acid sequence of SEQ ID NO: 125 and a light chain amino acid sequence of SEQ ID NO: 126. In certain embodiments, the N-terminal and/or C-terminal truncation is a truncation of the Fab-(scFv)₂ of a heavy chain amino acid sequence of SEQ ID NO: 127 and a light chain amino acid sequence of SEQ ID NO: 128. In certain embodiments, the N-terminal and/or C-terminal truncation is a truncation of the Fab-(scFv)₂ of a heavy chain amino acid sequence of SEQ ID NO: 129 and a light chain amino acid sequence of SEQ ID NO: 130. In certain embodiments, the N-terminal and/or C-terminal truncation is a truncation of the Fab-(scFv)₂ of a heavy chain amino acid sequence of SEQ ID NO: 131 and a light chain amino acid sequence of SEQ ID NO: 132. In certain embodiments, the N-terminal and/or C-terminal truncation is a truncation of the Fab-(scFv)₂ of a heavy chain amino acid sequence of SEQ ID NO: 133 and a light chain amino acid sequence of SEQ ID NO: 134. In certain embodiments, the N-terminal and/or C-terminal truncation is a truncation of the Fab-(scFv)₂ of a heavy chain amino acid sequence of SEQ ID NO: 135 and a light chain amino acid sequence of SEQ ID NO: 136. In certain embodiments, the N-terminal and/or C-terminal truncation is a truncation of the Fab-(scFv)₂ of a heavy chain amino acid sequence of SEQ ID NO: 137 and a light chain amino acid sequence of SEQ ID NO: 138. In certain embodiments, the Q at position 1 of the antigen binding proteins is removed which may prevent pyroglutamate formation and protects the protein to be clipped by proteases; in certain embodiments, this may lower the possibility of having different charge variants which may result challenging in the CMC process. An N-terminal R removal may in certain embodiments result in a lower pre-existing antibody response.

In certain embodiments, the variant sequence of the monovalent Fab-scFvs or bivalent Fab-(scFvs)₂ as described above comprises a pyroglutamate at amino acid position 1. In certain embodiments, the light chain of the antigen binding protein comprises pyroglutamate (pE) instead of the N-terminal glutamate.

In one embodiment, the antigen binding protein is glycosylated. Glycosylation refers to a process that attaches carbohydrates to proteins. In biological systems, this process is performed enzymatically within the cell as a form of co-translational and/or post-translational modification. A protein can also be chemically glycosylated. The carbohydrates may be N-linked to a nitrogen of asparagine or arginine side-chains; O-linked to the hydroxy oxygen of serine, threonine, tyrosine, hydroxylysine, or hydroxyproline side-chains; employ xylose, fucose, mannose, and N-acetylglucosamine attached to a phospho-serine; and/or adding mannose sugar to a tryptophan residue found in a specific recognition sequence. Glycosylation patterns may, e.g., be controlled by choosing appropriate cell lines, culturing media, protein engineering manufacturing modes and process strategies (see, HOSSLER, P. Optimal and consistent protein glycosylation in mammalian cell culture. Glycobiology 2009, vol. 19, no. 9, p. 936-949). In some embodiments, the glycosylation patterns of the antigen binding proteins described herein are modified to enhance ADCC and CDC effector function.

The antigen binding protein may be engineered to control or alter the glycosylation pattern, e.g., by deleting and/or adding of one or more glycosylation sites. The creation of glycosylation sites can e.g., be accomplished by introducing the corresponding enzymatic recognition sequence into the amino acid sequence of the antigen binding protein.

In certain embodiments, the (multispecific) antigen binding protein comprises a pyroglutamate (pE, pyrGlu, pyre or pGlu) instead of the N-terminal glutamine or the N-terminal glutamate. In certain embodiments, the light chain of the antigen binding protein comprises pyroglutamate (pE) instead of the N-terminal glutamine. In certain embodiments, the light chain of the antigen binding protein comprises pyroglutamate (pE) instead of the N-terminal glutamate. In certain embodiments, such pyroglutamate (pE) modification has no impact on the safety and/or efficacy of the (multispecific) antigen binding protein. In certain embodiments, the N-terminal glutamine (Q) of a light chain disclosed herein is clipped, e.g., to prevent pyroglutamate formation.

In some embodiments, the antigen binding protein is PEGylated. PEGylation may alter the pharmacodynamic and pharmacokinetic properties of a protein. Additionally, PEGylation may reduce the immunogenicity by shielding the PEGylated antigen binding protein from the immune system and/or alter its pharmacokinetics by, e.g., increasing the in vivo stability of the antigen binding protein, protecting it from proteolytic degradation, extending its half-life time and by altering its biodistribution. Typically, polyethylene-glycol (PEG) of an appropriate molecular weight is covalently attached to the protein. Similar effects may be achieved using PEG mimetics, e.g., HESylating, PASylating, or XTENylating the antigen binding protein. HESylation utilizes hydroxyethyl starch (“HES”) derivatives. During PASylation, the antigen binding protein is linked to conformationally disordered polypeptide sequences composed of the amino acids proline (P), alanine (A) and serine (S), and XTENylation employs a similar, intrinsically disordered XTEN-polypeptide.

In certain embodiments, the antigen binding protein (e.g., the multispecific antigen binding protein) is linked to or combined with a detectable label, a therapeutic agent or a PK modifying moiety. For example, the antigen binding protein can is labelled with or conjugated to a second moiety which attributes one or more ancillary functions to the antigen binding protein. For example, the second moiety may have an additional immunological effector function, be effective in drug targeting or useful for detection. The second moiety can, e.g., be chemically linked or fused genetically to the antigen binding protein using known methods in the art. As used herein, the term “label” refers to any substance or ion which is indicative of the presence of the antigen binding protein when detected or measured by physical or chemical means, either directly or indirectly. For example, the label may be directly detectable by, without being limited to, light absorbance, fluorescence, reflectivity, light scatter, phosphorescence, or luminescence properties, molecules or ions detectable by their radioactive properties or molecules or ions detectable by their nuclear magnetic resonance or paramagnetic properties. Examples of indirect detection include light absorbance or fluorescence; for example, various enzymes which cause appropriate substrates to convert, e.g., from non-light absorbing to light absorbing molecules, or from non-fluorescent to fluorescent molecules. A labelled antigen binding protein is particularly useful for in vitro and in vivo detection or diagnostic purposes. For example, an antigen binding protein labelled with a suitable radioisotope, enzyme, fluorophore or chromophore can be detected by radioimmunoassay (RIA), enzyme-linked immunosorbent assay (ELISA), or flow cytometry-based single cell analysis (e.g., FACS analysis), respectively. Similarly, the nucleic acids and/or vectors disclosed herein can be labeled for detection or diagnostic purposes, e.g., using labelled fragments thereof as probes in hybridization assays.

Non-limiting examples of second moieties include radioisotopes (35S, 32P, 14C, 18F, and/or 125I), apoenzymes, enzymes (e.g., alkaline phosphatase, horseradish peroxidase, beta-galactosidase and/or angiogenin), co-factors, peptide moieties (e.g., a HIS-tag), proteins (e.g. lectin, serum albumin), carbohydrates (e.g., mannose-6-phosphate tags), fluorophores (e.g., fluorescein isothiocyanate (FITC)), phycoerythrin, green/blue/red or other fluorescent proteins, allophycocyanin (APC), chromophores, vitamins (e.g., biotin), chelators, antimetabolites (e.g., methotrexate), toxins (e.g. a cytotoxic drug, or a radiotoxin).

In one aspect, the invention relates to drug conjugates (in particular antibody-drug conjugates ADCs) comprising the antigen binding proteins described herein, e.g., a monovalent or a multispecific antigen binding protein described herein (e.g., an antibody), conjugated to a toxin which further enhances efficient killing of specific cells, such as e.g., MHC-displayed SLLQHLIGL (SEQ ID NO: 139) positive cells. The toxin moiety is typically a small molecular weight moiety, such as MMAE/MMAF, DM1, chaliceamicin, anthracycline toxins, taxol, gramicidin D and/or colchicine, which may be linked via a peptide linker to the antigen binding protein. In certain embodiments, the ADC comprises a scFv comprising or consisting of the amino acid sequence of any of SEQ ID NOs: 93-103, or a variant as described herein. In certain embodiments, said variant is at least about 90%, 95%, 96%, 97%, 98% or 99% identical to the amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, or SEQ ID NO: 103, respectively. In certain embodiments thereof, the variant comprises the HCDR1 amino acid sequence of SEQ ID NO: 25, the HCDR2 amino acid sequence of SEQ ID NO: 26, the HCDR3 amino acid sequence of SEQ ID NO: 27, the LCDR1 amino acid sequence of SEQ ID NO: 28, the LCDR2 amino acid sequence of SEQ ID NO: 29, and/or the LCDR3 amino acid sequence of SEQ ID NO: 30.

The toxin may be conjugated non-site-specifically or site-specifically to the antigen binding protein. Non-site-specific conjugation typically involves the use of chemical linkers, e.g., with maleimide functionality, that mediate conjugation to lysine or cysteine amino acid side chains of the antigen binding protein or to the amino-group of the N-terminus. Site-specific conjugation may be achieved using chemical, chemo-enzymatic, or enzymatic conjugations known in the art, e.g., employing bifunctional linkers, bacterial transglutaminase or sortase enzymes, linkers allowing Pictet-Spengler chemistry on formyl-glycine forming enzyme modified antigen binding proteins, or glycan-remodeled antigen binding proteins.

### Methods of Administering Antigen Binding Proteins

Methods of preparing and administering antigen binding proteins of the disclosure (such as the multispecific antigen binding protein described above) as well as the nucleic acids described herein, the vectors described herein, the host cells described herein or the compositions described herein to a subject are well known to or are readily determined by those skilled in the art. The route of administration of the antigen binding proteins of the current disclosure may e.g., be oral, parenteral, by inhalation, or topical. The term parenteral as used herein includes intravenous, intraarterial, intraperitoneal, intramuscular, subcutaneous, rectal or vaginal administration. The term topical as used herein includes, but is not limited to, administration with liquid or solution eye drops, emulsions (e.g., oil-in-water emulsions), suspensions, and ointments.

While all these forms of administration are clearly contemplated as being within the scope of the current disclosure, a form for administration would be a solution for injection. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

Effective doses of the compositions of the present disclosure, for the treatment of the related conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

As previously discussed, the antigen binding proteins of the present disclosure (e.g., the multispecific antigen binding protein), conjugates or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent. As used herein, an “effective amount” of an agent, e.g., a pharmaceutical composition, refers to an amount effective to achieve the desired therapeutic or prophylactic result, at dosages and for periods of time as necessary.

In another aspect, the disclosure provides for the use of the antigen binding protein recited above, such as the bispecific antigen binding protein recited above, for preparing a pharmaceutical composition for treating a PRAME associated cancer in a subject.

In another aspect, the disclosure provides a pharmaceutical composition comprising the antigen binding proteins recited above, in particular the bispecific antigen binding protein recited above, and a pharmaceutically acceptable carrier.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the antigen binding proteins (such as the multispecific antigen binding protein) shall be held to mean an amount sufficient to achieve effective binding to an antigen and to achieve a benefit, e.g., to ameliorate symptoms of a disease or disorder or to detect a substance or a cell. In the case of tumor cells, the antigen binding proteins will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure (such as the multispecific antigen binding protein described above) may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antigen binding proteins of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of antigen binding proteins described in the current disclosure may prove to be particularly effective. Similarly, the nucleic acids described herein, the vectors described herein, the host cell cells described herein (in particular the immune cells bearing a CAR) or the compositions described herein may be administered to a human or other animal in accordance with the methods of treatment described above in an amount sufficient to produce a therapeutic or prophylactic effect.

“Efficacy” or “in vivo efficacy” as used herein refers to the response to a therapy by the pharmaceutical composition of the disclosure, using e.g., standardized response criteria. The success or in vivo efficacy of the therapy using a pharmaceutical composition of the disclosure refers to the effectiveness of the composition for its intended purpose, i.e., the ability of the composition to cause its desired effect. The in vivo efficacy may be monitored by established standard methods for the specific diseases. In addition, various disease specific clinical chemistry parameters and other established standard methods may be used.

In some embodiments, the compounds and cells described herein are administered in combination with one or more different pharmaceutical compounds. Generally, therapeutic use of the compounds and cells described herein may be in combination with one or more therapies selected from the group of antibody therapy, chemotherapy, cytokine therapy, dendritic cell therapy, gene therapy, hormone therapy, laser light therapy, radiation therapy or vaccine therapy.

### Methods of Treating Cancer

Provided herein are methods of treating cancer with the antigen binding proteins and in particular with the multispecific antigen binding proteins of the disclosure (e.g., an antigen binding protein comprising a Fab domain which binds a cell surface protein of an immune cell linked to a first and second pMHC antigen binding protein). The methods may be used to treat patients having any tumor type in which at least some of the cancer cells express a target peptide as disclosed herein displayed on a pMHC, such as MHC-displayed SLLQHLIGL (SEQ ID NO: 139). Such target peptide positive cancers or cancer cells can be assessed using any method known in the art, including, but are not limited to, detecting RNA expression levels or histological methods such as Immunohistochemistry (IHC).

In certain embodiments of the antigen binding protein of the disclosure, the target pMHC binding domain specifically targets an MHC restricted peptide derived of a tumor antigen.

In some embodiments, the target peptide positive cancer is a solid tumor and/or a hematological tumor, optionally wherein the cancer is selected from esophageal cancer, gastric adenocarcinoma, lung adenocarcinoma, and lung squamous cancer.

In one aspect, the upregulation of MHC-displayed SLLQHLIGL (SEQ ID NO: 139) expression on the cell surface is a biomarker for cancer.

In one aspect, the disclosure provides a method for killing a target cell comprising a major histocompatibility complex (MHC) presenting a neoantigen, the method comprising: a) contacting a plurality of cells comprising immune cells and the target cell with the antigen binding protein described herein, such as the multispecific antigen binding protein above, wherein said antigen binding protein specifically binds to the pMHC on the surface of the target cell and to CD3 on the surface of the immune cells; b) forming a specific binding complex through the antigen binding protein interactions with the target cells and the immune cells, thereby activating the immune cells; and c) killing the target cell with the activated immune cells.

In one aspect, the disclosure provides a method of treating cancer comprising the step of administering the antigen binding protein described herein, the nucleic acid described herein, vectors described herein, the host cells described herein or the pharmaceutical composition described herein, to a patient in need thereof.

In one aspect, the aforementioned antigen binding proteins (including antibody-drug conjugates), nucleic acids, vectors or host cells (in particular immune cells expressing CARs) or the vector, are useful as a medicament. Typically, such a medicament includes a therapeutically effective amount of a molecule or cell as provided herein. Accordingly, a respective molecule or host cell can be used for the production of a medicament useful in the treatment of one or more disorders, in particular disorders or diseases consisting of MHC-displayed SLLQHLIGL (SEQ ID NO: 139).

Also provided is the use of the aforementioned antigen binding proteins, nucleic acids, vectors or host cells (in particular immune cells expressing CARs) in the manufacture of a medicament.

In one aspect, a method of treating disorder or diseases consisting of MHC-displayed SLLQHLIGL (SEQ ID NO: 139) is provided. The method includes the steps of administering a pharmaceutically effective amount of a molecule or host cell as described herein, in particular the antigen binding proteins or a CAR expressing cell, to a subject in need thereof. In one embodiment, the pharmaceutical composition described above, which includes such pharmaceutically effective amount of the antigen binding protein, nucleic acid, vector or host cell (e.g., immune cell) is administered to the subject. The medicament referred to above may be administered to a subject.

In another aspect, the disclosure provides a method of treating a MHC-displayed SLLQHLIGL (SEQ ID NO: 139) positive cancers, in a patient in need thereof comprising administering to the patient a therapeutically effective amount of the antigen binding protein described herein, such as the multispecific antigen binding protein described herein, the CAR described herein, the host cell (e.g., immune cell) described herein, or the pharmaceutical composition recited above.

In certain aspects, patients eligible for treatment with an antagonist as described herein are selected based on RNA sequencing and/or immunohistochemistry (IHC), such as for detection of total target peptide.

The subject in need of treatment can be a human or a non-human animal. In typical embodiments, the subject is diagnosed with an MHC-displayed SLLQHLIGL (SEQ ID NO: 139) related disorder or may acquire such a disorder. In case of an animal model, the animal might be genetically engineered to develop MHC-displayed SLLQHLIGL (SEQ ID NO: 139) related disorder. An animal may also be genetically engineered in such a way that it shows the characteristics of MHC-displayed SLLQHLIGL (SEQ ID NO: 139) related disease.

In certain embodiments, disorders or diseases consisting of MHC-displayed SLLQHLIGL (SEQ ID NO: 139) expression is cancer. In certain embodiments, the cancer is a solid tumor. Optionally, the cancer is selected from endometrial cancer, melanoma, ovarian cancer, non-small cell lung cancer (NSCLC)-squamous, non-small cell lung cancer (NSCLC) non-squamous, NSCLC-adeno, SCLC, synovial sarcoma or TNBC. Optionally, the solid cancer is selected from the group consisting of esophageal carcinoma, lung adenocarcinoma, lung squamous cell carcinoma, ovarian carcinoma, skin cutaneous melanoma, thymoma, uterine corpus endometrial carcinoma and uterine carcinosarcoma.

### Use in Diagnostics and Detection Assays

An antigen binding protein as disclosed herein may be used for detection or diagnostic purposes in vivo and/or in vitro. For example, a wide range of immunoassays using antibodies for detecting the expression in specific cells or tissues are known to the skilled person. For such purposes, it may be advantageous to use an antigen binding protein connected to a detectable label, such as biotin.

In one embodiment, the described antigen binding proteins are useful for detecting the presence of a target peptide-MHC complex as described elsewhere herein in a sample. The detection may be for quantitative or qualitative purposes. The sample is preferably of biological origin, such as blood, urine, cerebrospinal fluid, biopsy, lymph and/or non-blood tissues. In certain embodiments, a biological sample comprises a cell or tissue from a human patient. In certain embodiments, the method includes contacting a biological sample with an antigen binding protein described herein, the CAR described herein, the immune cell described herein, the under conditions permissive for binding of the inhibitor to the target peptide-MHC and then detecting the inhibitor-target peptide-MHC and then detecting the inhibitor-target complex. Such method may be an in vitro or in vivo method. In some embodiments, such method is performed to select subjects eligible for therapy with the antigen binding protein described herein.

In one embodiment, the described antigen binding proteins are useful for detecting the presence of a target peptide-MHC complex as described elsewhere herein in a sample (e.g., from a patient). In certain embodiments, such method comprises the steps of
(i) providing a sample from a patient;
(ii) adding a target peptide detection antibody to the sample;
(iii) incubating said detection antibody and the sample; and
(iv) detecting said detection antibody bound to the sample.

In certain embodiments, step (iv) is followed by the step of
(v) selecting the patient for treatment with a antagonist as disclosed herein if said detection antibody is bound by the sample.

### Kits

Also contemplated are kits comprising at least one nucleic acid library or antigen binding protein, such as an antibody, including the multispecific antigen binding protein, or the pharmaceutical composition as described herein, typically together with a packaged combination of reagents with instructions. In one embodiment, the kit includes a composition containing an effective amount of said antigen binding protein in unit dosage form. Such kit may comprise a sterile container comprising the composition; non-limiting examples of such containers include, without being limited to, vials, ampoules, bottles, tubes, syringes, blister-packs. In some embodiments, the composition is a pharmaceutical composition and the containers are made of a material suitable for holding medicaments. In one embodiment, the kit may comprise in a first container the antigen binding protein in lyophilized form and a second container with a diluent (e.g., sterile water) for reconstitution or dilution of the antigen binding protein. In some embodiments, said diluent is a pharmaceutically acceptable diluent. In one embodiment, the kit is for diagnostic purposes and the antigen binding protein is formulated for diagnostic applications. In one embodiment, the kit is for therapeutic purposes and the antigen binding protein is formulated for therapeutic applications.

Typically, the kit will further comprise a separate sheet, pamphlet or card supplied in or with the container with instructions for use. If the kit is intended for pharmaceutical use, it may further comprise one or more of the following: information for administering the composition to a subject having a related disease or disorder and a dosage schedule, description of the therapeutic agent, precautions, warnings, indications, counter-indications, overdosage information and/or adverse reactions.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Production of HLA-A*02:01/PRAME Antigen for Animal Immunization

Genes of the MHC class I heavy chain, HLA-A*02:01 and β2m domain were each cloned into a pET-24D (+) vector using standard molecular biology techniques (J Biol Chem. 1995 Jan. 13; 270(2):971-7). E. coli BL-21 (DE3) cells were transformed with the expression vectors via electroporation. Protein expression was performed for 16-18 hours at 37° C. with 220 rpm shaking in MagicMedium (Invitrogen), as described by the supplier. Cells were harvested, resuspended in TBS and lysed via lysozyme treatment and sonication. Inclusion bodies were washed three times with TBS supplemented with 0.5% LDAO and twice with TBS. Such prepared inclusion bodies were solubilized using 8 μM urea, 100 mM Tris-HCl pH 8 buffer at ratio of 6 mL buffer per 1 g inclusion body pellet. Refolding and purification of the solubilized HLA-A*02:01 extracellular domain (SEQ ID NO: 23), β2m (SEQ ID NO: 24) and PRAME peptide SLLQHLIGL (peptides & elephants, SEQ ID NO: 139) were performed essentially as described by Rodenko et al. (2006). The purity of HLA-A*02:01/PRAME complex (pMHC) was assessed by SDS-PAGE and SE-HPLC. Amino acid sequences of each pMHC component are recited in Table 4.

TABLE 4
Amino acid sequences of HLA-A*02:01/
PRAME antigen components

| Sequence ID | Sequence |
|---|---|
| HLA-A*02:01 | GSHSMRYFFTSVSRPGRGEPRFIAVGYVDDTQ |
| extracellular | FVRFDSDAASQRMEPRAPWIEQEGPEYWDGET |
| domain | RKVKAHSQTHRVDLGTLRGYYNQSEAGSHTVQ |
| SEQ ID NO: 23 | RMYGCDVGSDWRFLRGYHQYAYDGKDYIALKE |
| | DLRSWTAADMAAQTTKHKWEAAHVAEQLRAYL |
| | EGTCVEWLRRYLENGKETLQRTDAPKTHMTHH |
| | AVSDHEATLRCWALSFYPAEITLTWQRDGEDQ |
| | TQDTELVETRPAGDGTFQKWAAVVVPSGQEQR |
| | YTCHVQHEGLPKPLTLRWE |
| human ß2m | IQRTPKIQVYSRHPAENGKSNFLNCYVSGFHP |
| SEQ ID NO: 24 | SDIEVDLLKNGERIEKVEHSDLSFSKDWSFYL |
| | LYYTEFTPTEKDEYACRVNHVTLSQPKIVKWD |
| | RDM |
| PRAME peptide | SLLQHLIGL |
| SEQ ID NO: 139 | |

### Example 2—Generation of Antibodies Against HLA-A*02:01/PRAME

To generate antibodies that recognize the HLA-A*02:01/PRAME, three New Zealand white rabbits were immunized with the recombinantly produced HLA-A*02:01/PRAME antigen. Immunization was performed by intrasplenic injection. Each animal received five injections of the HLA-A*02:01/PRAME supplemented with the complete or incomplete Freund's adjuvant. Immune response was tested by ELISA to confirm the presence of anti-HLA-A*02:01/PRAME antibodies in the serum. PBMCs and spleen lymphocytes were isolated from the sacrificed animals.

scFv antibody cDNA libraries were generated based on the RNA extracted from the PBMCs and spleen lymphocytes via RT-PCR. Coding sequences for the variable light (VL) and heavy (VH) domains were amplified separately via PCR with an introduction of flanking restriction sites on the VL and VH DNA fragments. Amplified DNA was inserted into a phagemid vector to generate the complete scFv upstream and in-frame of the amber stop codon and bacteriophage M13 pIII gene, using standard molecular biology protocols. Phagemid vectors containing the immune libraries were transformed into E. coli TG1 electrocompetent cells. Finally, two antibody libraries were generated, comprising a diversity of 1.14×109 with a sequence accuracy of 95.8% for the kappa-based library and 3.61×10⁸ with an accuracy of 95.8% for the lambda-based library. Prior screening, kappa-based libraries were optimized by removal of the free cysteine at position 80, as described in WO2022190007A1.

Rabbit-derived immune libraries were screened for HLA-A*02:01/PRAME specific binders. Briefly, three rounds of phage display biopanning were performed. Each round consisted of a deselection step against either a pool of HLA-A*02:01 complexes displaying target-unrelated peptides (Table 5) or a pool of HLA-A*02:01 complexes loaded with peptides showing high sequence identity to the target, i.e., at least 55% (Table 6). The deselection step was followed by selection on target complex HLA-A*02:01/PRAME. Screening of binders was performed with a monoclonal phage ELISA after biopanning round two and three in which binding to the target complex HLA-A*02:01/PRAME, unrelated complexes and complexes displaying peptides of high sequence identity to the target was quantified. Signal ratios of HLA-A*02:01/PRAME binding to unrelated peptide pool binding and to the similar peptide pool binding were determined to identify hits binding specifically to the target. A monoclonal phage titration ELISA of hits was performed to determine concentration-dependent and specific binding to the target complex. Hits capable of binding HLA-A*02:01/PRAME and showing no binding to the control complexes were selected for further characterization and produced in a Fab-scFv bispecific format, where the Fab comprised a CD3 binding moiety and the scFv comprised the pMHC binding moiety.

TABLE 5
Amino acid sequences of target unrelated peptides.

| HLA type | Peptide sequence | Peptide origin | Sequence ID |
|---|---|---|---|
| HLA-A*02:01 | GIDDLHISL | SYNE2 | SEQ ID NO: 148 |
| HLA-A*02:01 | GLDGHSHLV | SEMA4A | SEQ ID NO: 149 |
| HLA-A*02:01 | GLDSYLPEL | DOCK9 | SEQ ID NO: 150 |
| HLA-A*02:01 | GLYEGEFTL | MBTPS1 | SEQ ID NO: 151 |
| HLA-A*02:01 | GQYELVHTL | DSTYK | SEQ ID NO: 152 |
| HLA-A*02:01 | GMTELYFQL | PRKDC | SEQ ID NO: 153 |
| HLA-A*02:01 | GIADLAHLL | BUB1B | SEQ ID NO: 154 |
| HLA-A*02:01 | GLYSKMNGL | GATA6 | SEQ ID NO: 155 |
| HLA-A*02:01 | GVIDAYMTL | PRKDC | SEQ ID NO: 156 |
| HLA-A*02:01 | GVYDGEEHSV | MAGEB2 | SEQ ID NO: 157 |
| HLA-A*02:01 | SLYDSKIWTT | POT1 | SEQ ID NO: 158 |
| HLA-A*02:01 | GQYEGHEAEL | RBM33 | SEQ ID NO: 159 |
| HLA-A*02:01 | AQYDGKGVGL | COL1A2 | SEQ ID NO: 160 |
| HLA-A*02:01 | ALDVYNGLL | ACPP | SEQ ID NO: 161 |
| HLA-A*02:01 | GVRGRVEEI | BCR-ABL | SEQ ID NO: 162 |
| HLA-A*02:01 | YVLDHLIVV | BRLF1 | SEQ ID NO: 163 |
| HLA-A*02:01 | GLLSLEEEL | bZIP factor | SEQ ID NO: 164 |
| HLA-A*02:01 | LLLGPLGPL | Heparanase | SEQ ID NO: 165 |
| HLA-A*02:01 | VLEETSVML | IE-1 | SEQ ID NO: 166 |
| HLA-A*02:01 | VLFGLGFAI | IGRP | SEQ ID NO: 167 |
| HLA-A*02:01 | HLVEALYLV | Insulin | SEQ ID NO: 168 |
| HLA-A*02:01 | SLLFLLFSL | MSLN | SEQ ID NO: 169 |
| HLA-A*02:01 | SLLMWITQV | NY-ESO-1 | SEQ ID NO: 170 |
| HLA-A*02:01 | VLAGGFFLL | PSMA | SEQ ID NO: 171 |
| HLA-A*02:01 | YLIELIDRV | TACE | SEQ ID NO: 172 |
| HLA-A*02:01 | YMDGTMSQV | Tyrosinase | SEQ ID NO: 173 |
| HLA-A*02:01 | GLADQLIHL | Vif | SEQ ID NO: 174 |
| HLA-A*02:01 | LIDQYLYYL | VP1 | SEQ ID NO: 175 |
| HLA-A*02:01 | ALVEMGHHA | Vpu | SEQ ID NO: 176 |
| HLA-A*02:01 | SLGEQQYSV | WT1 | SEQ ID NO: 177 |
| HLA-A*02:01 | VLDFAPPGA | WT1 | SEQ ID NO: 178 |
| HLA-A*02:01 | YLNDHLEPWI | BCL-X | SEQ ID NO: 179 |
| HLA-A*02:01 | SLYNTVATLY | Gag | SEQ ID NO: 180 |
| HLA-A*02:01 | ALWPWLLMAT | RNF43 | SEQ ID NO: 181 |
| HLA-A*02:01 | FLPSPLFFFL | TARP-2M | SEQ ID NO: 182 |
| HLA-A*02:01 | ALLTSRLRFI | Telomerase | SEQ ID NO: 183 |
| HLA-A*02:01 | YLQVNSLQTV | Telomerase | SEQ ID NO: 184 |
| HLA-A*02:01 | ALYTGRLPEA | Synthetic | SEQ ID NO: 185 |

TABLE 6
Amino acid sequences of peptides displaying high sequence
identity to the target.

| HLA type | Peptide sequence | Peptide origin | Sequence ID |
|---|---|---|---|
| HLA-A*02:01 | SLLQHVLLL | HDAC5 | SEQ ID NO: 186 |
| HLA-A*02:01 | SLLKNLIYL | LRRC70 | SEQ ID NO: 187 |
| HLA-A*02:01 | SLIQHLEEI | DDX60L | SEQ ID NO: 188 |
| HLA-A*02:01 | SLSQELVGV | ZNF318 | SEQ ID NO: 189 |
| HLA-A*02:01 | VLLHHQIGL | IFIT1 | SEQ ID NO: 190 |
| HLA-A*02:01 | SLLQKQIML | VPS13B | SEQ ID NO: 191 |
| HLA-A*02:01 | SVLGALIGV | CCDC51 | SEQ ID NO: 192 |
| HLA-A*02:01 | SILQTLILV | TMED9 | SEQ ID NO: 193 |
| HLA-A*02:01 | KLLKNLIGI | GIMAP8 | SEQ ID NO: 198 |
| HLA-A*02:01 | KLARHFIGL | KCNG3 | SEQ ID NO: 199 |
| HLA-A*02:01 | YLQQHVAGL | TSC22D2 | SEQ ID NO: 200 |
| HLA-A*02:01 | YMNNDLIGL | TUBG2 | SEQ ID NO: 201 |
| HLA-A*02:01 | SLIRRIIGL | BBS10 | SEQ ID NO: 202 |
| HLA-A*02:01 | NLAEKLIGV | RALGAPB | SEQ ID NO: 203 |

### Example 3—Production of pMHC-Targeting T Cell Engagers

Monovalent bispecific antigen binding proteins in Fab-scFv format were expressed by transient co-transfection in HEK293-6E cells. Cells were cultured in suspension using polyethylenimine (PEI 40 kD linear). HEK293-6E cells were seeded at 1.7×10⁶ cells/mL in Freestyle F17 medium supplemented with 2 mM L-Glutamine and 25 ug/mL G418. DNA encoding the light and heavy chains of the molecules and PEI were added separately to 50 μL/mL medium without supplement. Both fractions were mixed at 1:2.5 DNA:PEI ratio, vortexed and rested for 15 minutes. Cells and DNA/PEI mixture were combined (1 pg DNA/mL cells) and incubated at 37° C., 5% CO2, 80% RH. After 24 hours, cells were supplemented with Tryptone N1 at 25 μL/mL production volume. After 7 days, cells were harvested by centrifugation and the supernatant was sterile filtered. The antigen binding proteins were purified by an affinity chromatography from the supernatant. Supernatant was loaded on a protein CH column (Thermo Fisher Scientific) equilibrated with 6 column volumes (CVs) of PBS (pH 7.4). After a washing step with the same buffer, protein was eluted from the column by step elution with 100 mM citric acid (pH 3.0). Fractions with the desired antigen binding protein were immediately neutralized by 1 μM Tris Buffer (pH 9.0) at 1:10 ratio. Size exclusion chromatography was performed as an additional purification step. Samples were run on the Superdex 200 10/300 GL column with PBS (pH7.4) as a running buffer. Collected fractions were analyzed by SE-HPLC for monomer content and pooled accordingly. Final protein purity was assessed by SDS-PAGE and SE-HPLC.

Bivalent bispecific antigen binding proteins in Fab-(scFv)₂ format were produced by transient co-transfection in CHO-K1 cells. The genes for heavy and light chains were expressed using a 2:1 vector ratio in shake flask cultures for 7 days. The target proteins were captured from the clarified, sterile-filtered culture supernatants by affinity chromatography (CH column, Thermo Fisher Scientific). The captured antigen binding proteins were further polished by strong cation exchange chromatography (CEC) over a Source 30S resin (Cytiva) and hydrophobic interaction chromatography (HIC) over a Toyopearl PPG-600M resin (Tosoh Bioscience). An Amicon stirred cell (Merck) was applied for transferring the target proteins into the final buffer (130 mM NaCl, 10 mM sodium phosphate, pH 6.5).

### Example 4—Humanization of Rabbit-Derived Antigen Binding Proteins

The anti-PRAME scFv of the most promising hit M2638 was humanized. Human germlines IMGT_hVH_3_66 and IMGT_hVx_1-39, displaying high sequence identity to VH and VL amino acid sequences of M2638 scFv were selected as CDR acceptor scaffolds. Rabbit-originating framework region 4 was replaced by the IGHJ1*01 and IGKJ1*01 human junction gene sequences in the VH and VL, respectively. Bispecific antibody fragment constructs were generated using a humanized sp34 variant as a CD3 engaging moiety and comprised Fab-scFv and Fab-(scFv)₂ formats monovalent for CD3 binding via the Fab domain and mono- or bivalent for PRAME binding through the scFv domain. M2913, M3076, M3182, M3202, M3283, M3284, M3285, M3286, M3287 and M3293 comprised humanized variants of M2638 with varying number of rabbit-originating residues in a monovalent HLA-A*02:01/PRAMExCD3 bispecific Fab-scFv format. M3134, M3167, M3275, M3279, M3288, M3289, M3290, M3291, M3292 and M3326 were generated based on the respective monovalent binders listed above and comprised HLA-A*02:01/PRAMExCD3 bispecifics bivalent for the PRAME antigen in a Fab-(scFv)₂ format.

### Example 5—Affinity Characterization of HLA-A*02:01/PRAMExCD3 Antigen Binding Proteins

Recombinantly produced hits in Fab-scFv format, as described in Example 4, were evaluated for their ability to bind HLA-A*02:01/PRAME antigen. Binding characterization was performed by surface plasmon resonance (SPR) using a Biacore™ 8k (Cytiva) device. Briefly, 3000 RU of Biotin CAPture reagent (Cytiva) was immobilized on a Series S Sensor Chip CAP (Cytiva) and the biotinylated ligand HLA-A*02:01/PRAME (SEQ ID NO: 68) was diluted in HPS-EP+ running buffer (TEKNOVA) and captured for 300 s at a flow rate of 2 μL/min at flow cell 2 resulting in a ligand capture level of >100 RU. Subsequently, at least three consecutive analyte injections were performed at suitable concentrations applying the single-cycle-kinetics mode at a flow rate of 30 μL/min with an association time of 90 s and the dissociation time set to >400 s. The chip surface was regenerated according to the manufacturer's instructions. After reference flow cell signal and blank injections subtraction, the data was fit to a 1:1 Langmuir binding model to determine the association rate constant kₐ and dissociation rate constant k_{d} and calculate the equilibrium dissociation constant K_{D} (k_{d}/kₐ), also denoted as affinity, using the Biacore™ Insight Evaluation software. The resulting K_{D}-values are shown in Table 7. All tested molecules showed high-affinity binding to the PRAME target with K_{D} values ranging from 0.04 nM to 1.2 nM.

TABLE 7
K_{D}-values (nM) of M2638 and M2638-derived humanized antigen
binding proteins to the target antigen HLA-A*02:01/PRAME.

| | Affinity to HLA- |
|---|---|
| | A*02:01/PRAME, |
| Molecule | K_{D} (nM) |
| M2638 | 0.23 |
| M2913 | 0.66 |
| M3076 | 0.07 |
| M3182 | 0.04 |
| M3202 | 0.04 |
| M3283 | 0.09 |
| M3284 | 0.27 |
| M3285 | 1.3 |
| M3286 | 1.5 |
| M3287 | 0.57 |
| M3293 | 0.05 |

### Example 6—In Vitro Characterization of Potency and Safety of Rabbit-Derived HLA-A*02:01/PRAMExCD3 Bispecific Antibody Hit

Cancer cell killing mediated by M2638 and the bivalent variant thereof M2873 was analyzed in a time-resolved manner using the IncuCyte S3 system. Briefly, a selection of HLA-A*02:01-positive PRAME-positive (NCI-H1703, OV56 and/or NCI-H1755) and HLA-A*02:01-positive PRAME-negative (TYK-nu, SW620 and/or Colo678) cancer cells were transduced with Nuclight Red lentivirus (Sartorius) to stably express the mKate2 fluorescent protein. Cancer cells were seeded at the density of 1.5×10³ cells per well in a sterile 384-well flat bottom adhesion tissue culture plate overnight at 37° C. and 5% CO2 in an incubator. Molecules M2638 and M2873 were added at the indicated concentrations (range of 0.005 nM-100 nM). PBMCs were added as effector cells to each well at an E:T ratio of 10:1. The plate was imaged by fluorescent microscopy to monitor cell growth for 72 h. The degree of cell killing was quantified by comparing the fold growth ratio of fluorescent target cancer cells over time, relative to their number at time 0. As depicted in FIG. 1, both the monovalent molecule M2638 and the bivalent M2873 showed a very specific cancer cell killing of the PRAME-positive cancer cells and no killing of the PRAME-negative cancer cells.

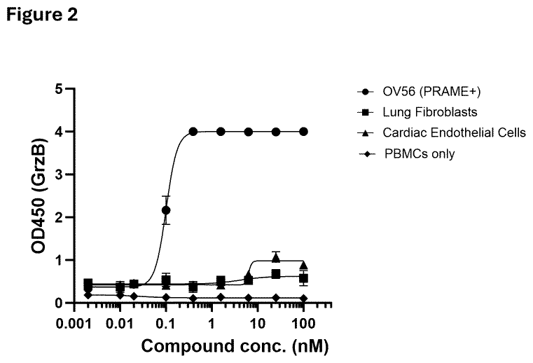

Safety of M2638 was tested on human primary cell types expressing HLA-A*02:01. Tested cells included lung fibroblasts (NHLF_76039) and cardiac microvascular endothelial cells (HCMEC_147). Cells were prepared in assay medium (RPMI 1640 containing 10% FBS and 1% penicillin-streptomycin) and plated at 20,000 cells per well in a volume of 50 μL assay medium. PBMCs effector cells were plated at 100,000 cells per well in a volume of 50 μL assay medium. Varying concentrations of compound M2638 (range of 0.05 nM to 100 nM) were added to the plated wells in 15 μL assay volume. The final assay medium was made up to 150 μL per well. All reactions were performed in duplicates. The plates were incubated for 24 h at 37° C./5% CO₂. Supernatants were collected and analyzed by human Granzyme B ELISA kit (MabTech) according to the manufacturer's instructions. The upper limit of quantification (ULOQ) was determined in each assay with an internal standard and the Granzyme B concentration in every sample was calculated according to the dilution of the cell supernatant. HLA-A*02:01-positive and PRAME-positive cell line OV56 served as a positive control and PBMCs only with the compound served as negative control. Corresponding results are depicted in FIG. 2. M2638 showed no reactivity and therefore a safe profile on the tested primary human cells.

### Example 7—Production of Comparator Molecules: A Soluble TCR×Anti-CD3 Fusion Protein (Comparator 1) and a Bispecific T Cell Engaging Receptor TCR×Anti-TCRα/β CD3×Fc Fusion Protein (Comparator 2)

DNA sequences encoding extracellular regions of the alpha (SEQ ID NO: 194) and beta (SEQ ID NO: 195) chains of soluble TCR×anti-CD3 fusion (Comparator 1) were separately cloned into pET-24D(+) vector using standard molecular biology techniques (J Biol Chem. 1995 Jan. 13; 270(2):971-7). E. coli BL-21 (DE3) were transformed with the expression vectors according to the supplier's protocols. Protein expression was performed for 16-18 hours at 37° C. with 220 rpm shaking in MagicMedium (Invitrogen), as described by the supplier. Cells were harvested, resuspended in TBS and lysed via lysozyme treatment and sonication. Inclusion bodies were washed twice with TBS supplemented with Triton-X100 (50 mM Tris-HCl pH 8.1, 0.5% Triton-X100, 100 mM NaCl, 10 mM NaEDTA) and twice with TBS (50 mM Tris, 100 mM NaCl, 10 mM EDTA, pH 8.1). Such prepared inclusion bodies were solubilized in a denaturing buffer (9M Urea, 0.5M Gua, 25 mM Tris, 1.25 mM EDTA, pH 8.1). Solubilized inclusion bodies from alpha chain and beta chain-anti-CD3 scFv fusion were combined and mixed with reducing agent at final DTT concentration of 20 mM. Solubilized and reduced inclusion bodies were slowly mixed with the refolding buffer (4M Urea, 400 mM L-Arg, 2 mM EDTA, 100 mM Tris, 10 mM L-Cysteine, 2.5 mM L-Cystine, pH 8.1) and incubated at room temperature overnight. Molecule was captured from a diluted and pH adjusted refolding solution by anion exchange chromatography using POROS 50HQ column. Molecule was eluted by applying a gradient of 0-500 mM NaCl in 20 mM Tris pH 8.1 over 50 column volumes on Akta® purifier device (Cytiva). Size exclusion chromatography was performed as an additional purification step. Samples were run on HiLoad Superdex 75, 26/600 column with PBS (pH7.4) as a running buffer. Collected fractions were analyzed by SE-HPLC for monomer content and pooled accordingly. Final protein purity was assessed by SDS-PAGE and SE-HPLC.

Bispecific T cell engaging receptor TCR×anti-T-cell×Fc fusion protein (Comparator 2, SEQ ID NO: 196, SEQ ID NO: 197) was expressed by transient co-transfection in CHO-K1 cells. The molecule expression was performed in shake flask cultures for 7 days. The target proteins were captured from the clarified, sterile-filtered culture supernatants by affinity chromatography (HiTrap PrismA, GE Healthcare). Size exclusion chromatography was performed as an additional purification step. Samples were run on Superdex 200 Increase 10/300 GL column with PBS (pH7.4) as a running buffer. Collected fractions were analyzed by SE-HPLC for monomer content and pooled accordingly. Final protein purity was assessed by SDS-PAGE, SE-HPLC and CEX-HPLC.

### Example 8—In Vitro Characterization of Potency and Safety of Humanized and Optimized HLA-A*02:01/PRAMExCD3 Bispecific Antibodies

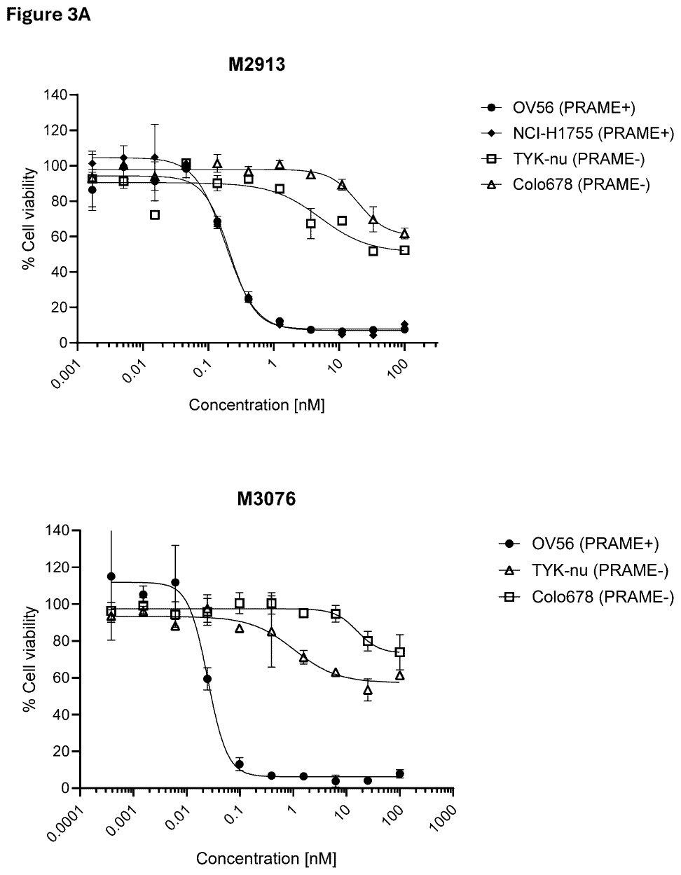

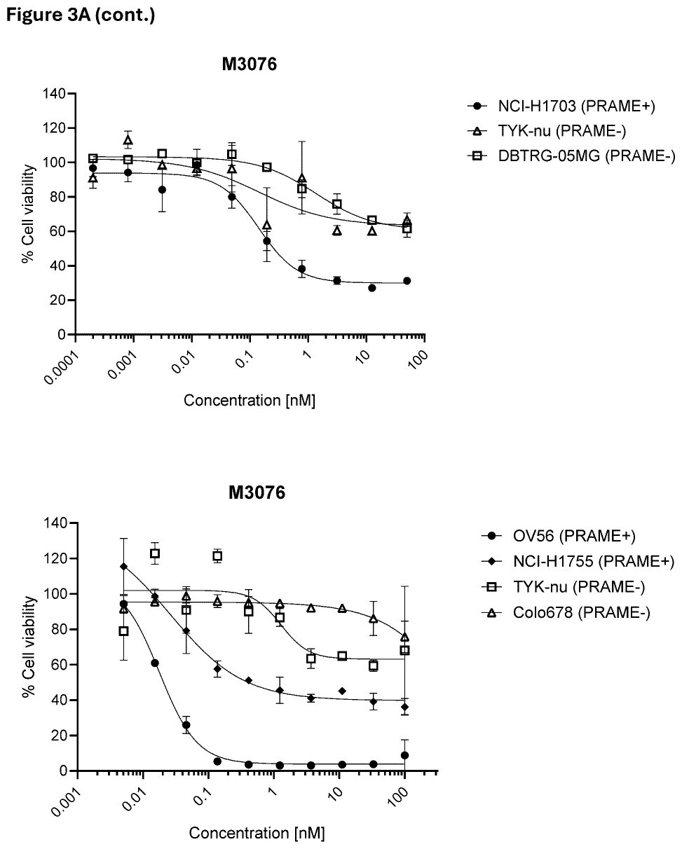

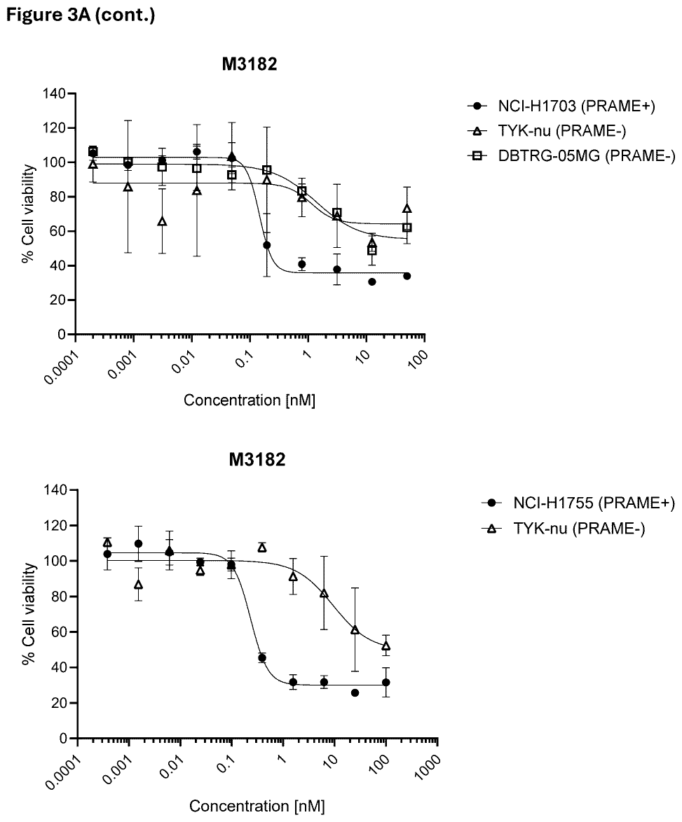

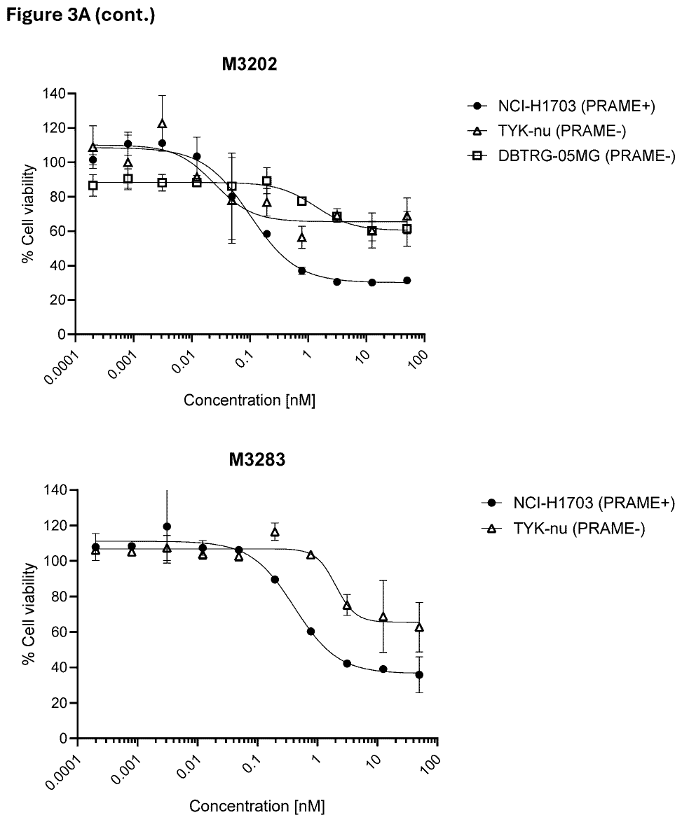

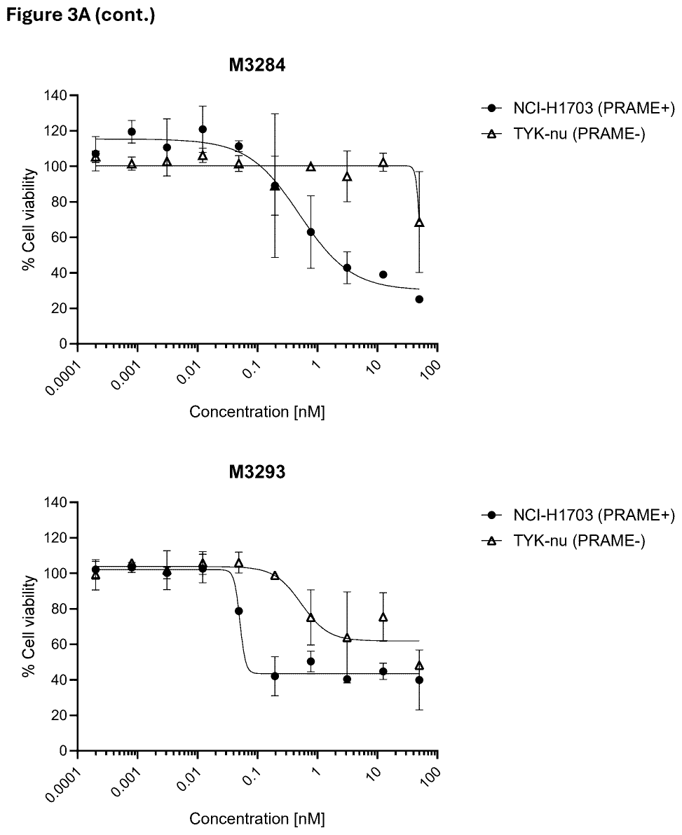

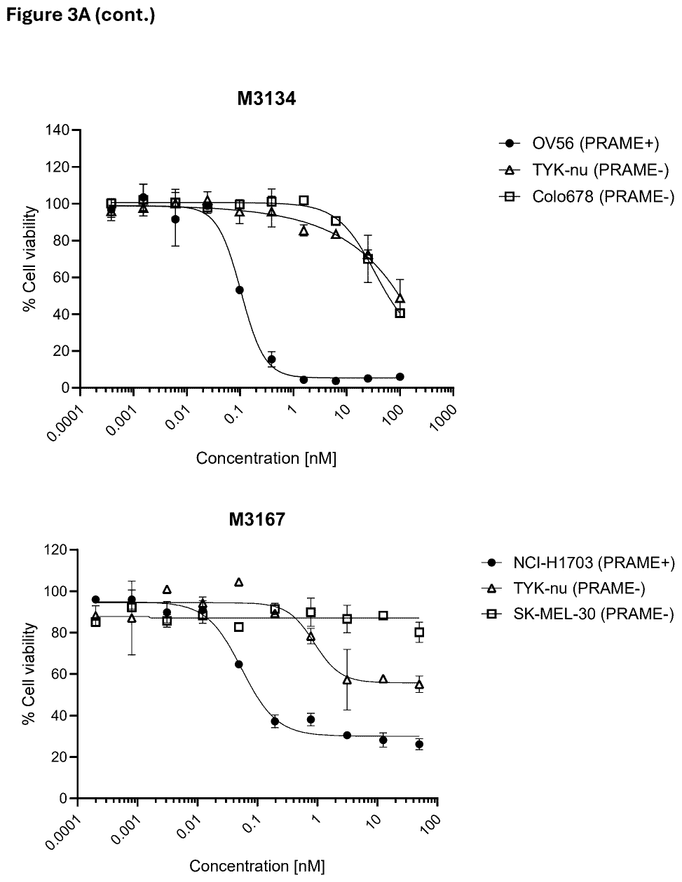

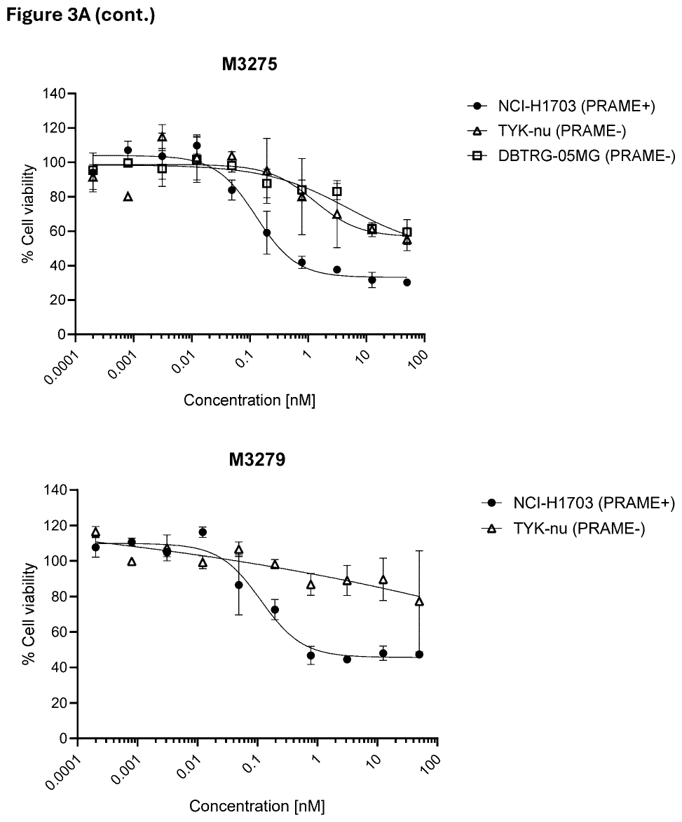

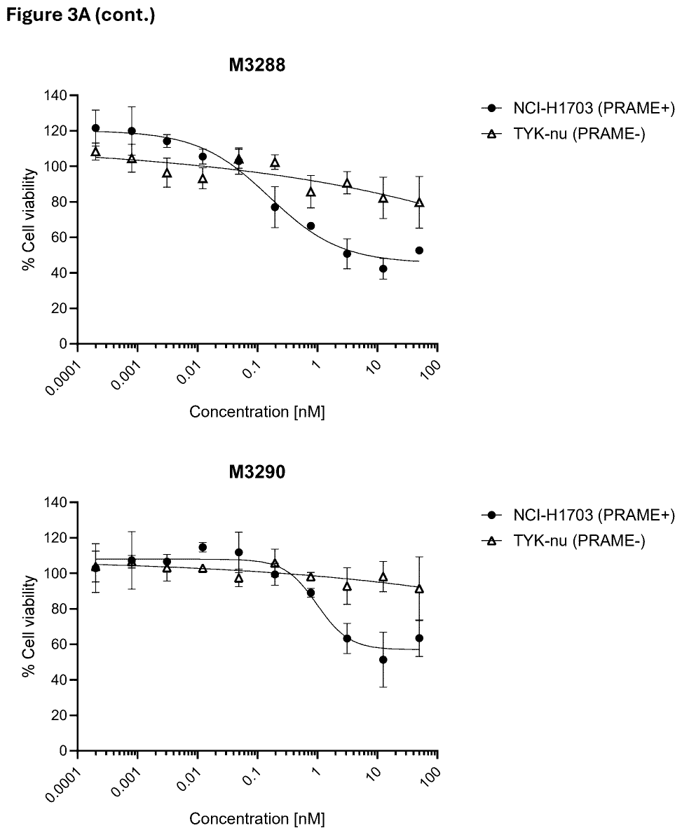

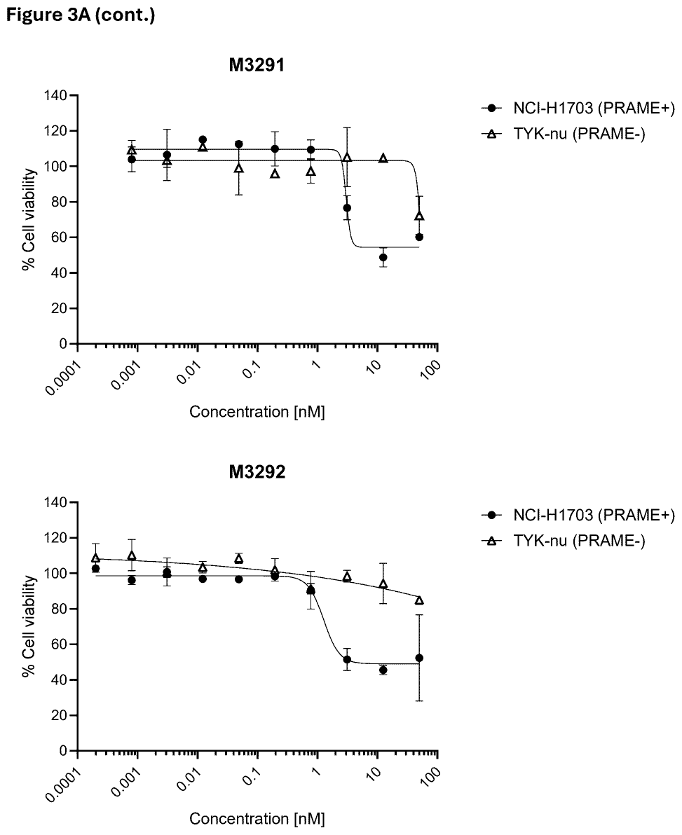

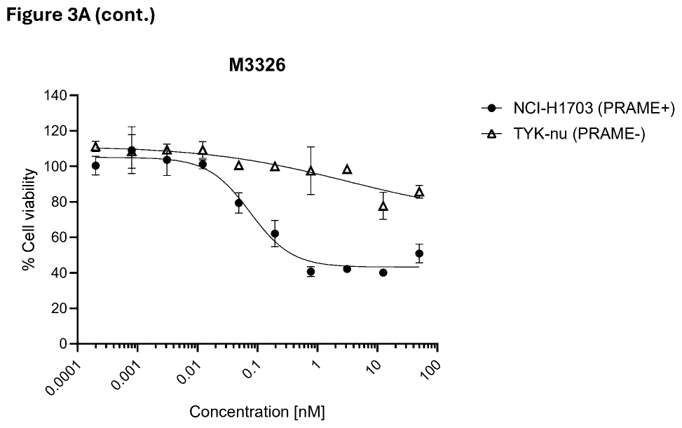

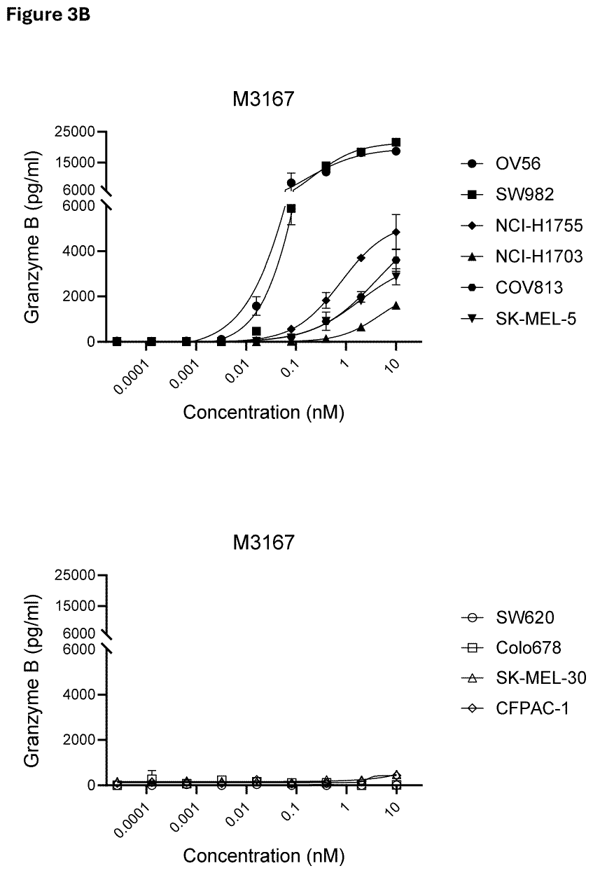

Humanized and optimized variants of the rabbit hit M2638 in a bispecific format monovalent (Fab-scFv) and bivalent (Fab-(scFv)₂) for HLA-A*02:01/PRAME binding were characterized for potency and safety in in vitro assays. Specific cancer cell killing was evaluated in a time-resolved manner using the IncuCyte S3 system. Tested cancer cell lines included NCI-H1703, NCI-H1755, OV56 and/or EFO-27, as a representation of HLA-A*02:01-positive PRAME-positive cancer cells, and TYK-nu, DBTRG-05MG, SK-MEL-30 and/or Colo678, as a representation of HLA-A*02:01-positive PRAME-negative cancer cells. The cell killing assay was performed essentially as described in Example 6. The molecules M2913, M3076, M3182, M3202, M3283, M3284, M3293, M3134, M3167, M3275, M3279, M3288, M3289, M3290, M3291, M3292 and M3326 were tested at different concentration ranges starting from 0.2 pM and up to 100 nM. All tested molecules showed specific cancer cell killing of PRAME-positive cell lines and good safety on PRAME-negative cancer cell lines (FIG. 3A).

Furthermore, M3167 potency was tested by Granzyme B release and the cell assay was performed essentially as described in Example 6. An extended panel of cell lines was used and included NCI-H1703, NCI-H1755, OV56, SW982, COV813 and/or SKMEL-5 as a representation of HLA-A*02:01-positive PRAME-positive cancer cells, and SK-MEL-30, Colo678, SW620 and/or CFPAC-1 as a representation of HLA-A*02:01-positive PRAME-negative cancer cells. The tested concentration range of compound M3167 was 0.026 pM to 10 nM. M3167 showed high Granzyme B release on PRAME-positive cell lines but no reactivity on PRAME-negative cancer cells representing the strong potency and good safety profile of the molecule (FIG. 3B).

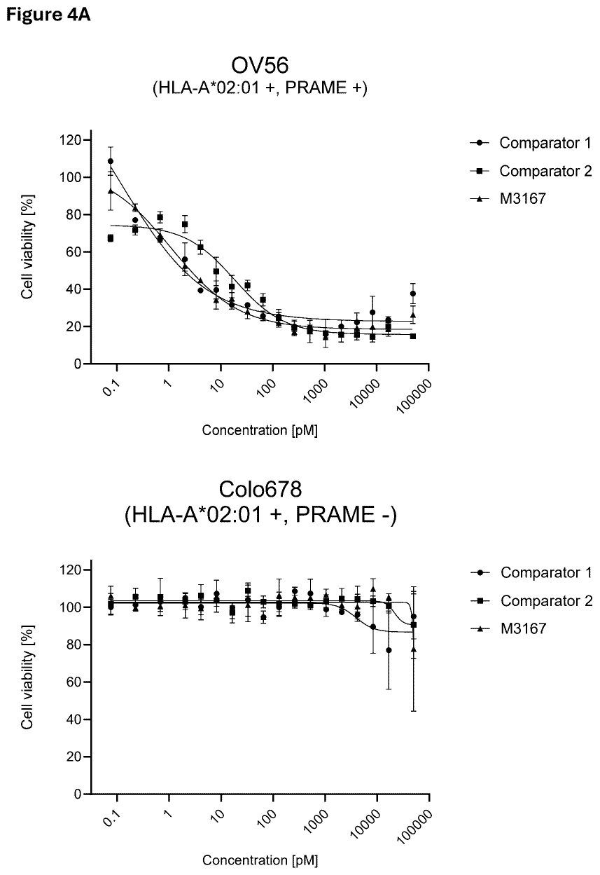

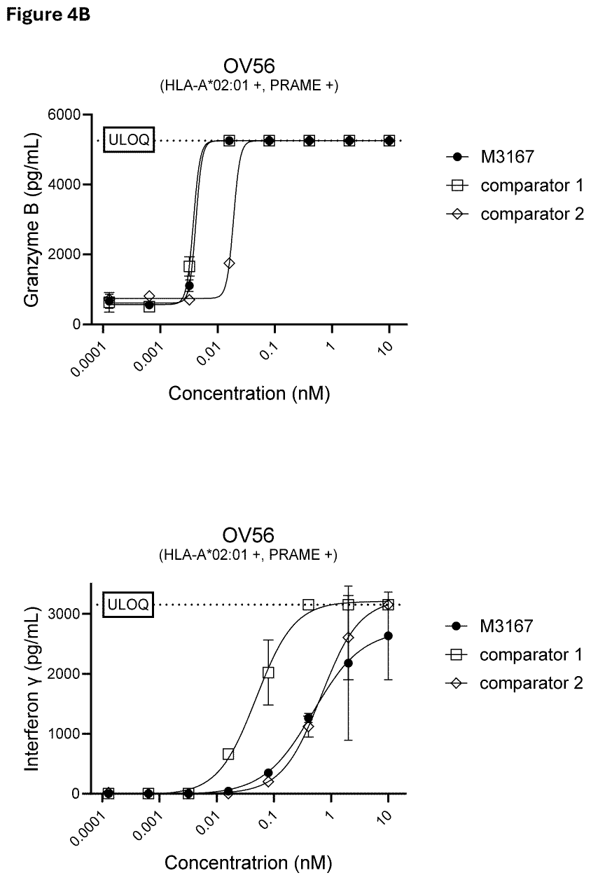

The potency of tested molecules correlated well with the respective affinities of the monovalent compounds measured in SPR for the HLA-A*02:01/PRAME antigen (Example 5). In addition, bivalent T cell engager M3167 was compared to two comparator antigen binding molecules, i.e., Comparator 1 and Comparator 2. Comparator 1 is composed of a soluble affinity-enhanced TCR with binding specificity for the same HLA-A*02:01/PRAME antigen with a K_{D} value of about 130 pM, linked to an anti-CD3 scFv with a binding affinity of about 1 nM. Comparator 2 is a soluble affinity-enhanced TCR with binding specificity for the same HLA-A*02:01/PRAME antigen with a K_{D} value of about 300 pM, linked with an anti-TCRα/β CD3 Fv for T cell engagement and Fc domain for extended serum circulation half-life. Both comparators are monovalent for the target pMHC and CD3 or TCRα/β CD3, while the dual engagers are bivalent for the target pMHC and monovalent for CD3. M3167, Comparator 1 and Comparator 2 were tested in an in vitro cancer cell killing assay using IncuCyte S3 system, according to the protocol described in detail in Example 6. M3167, Comparator 1 and Comparator 2 were tested at a concentration range of 0.07 pM-50 nM. The tested cancer cells included OV56 as an example of HLA-A*02:01-positive PRAME-positive cancer cells and Colo678 as an example of HLA-A*02:01-positive PRAME-negative cancer cells. M3167 showed a very potent killing of the PRAME-positive cancer cells OV56, comparable to Comparator 1 and more potent than Comparator 2 (FIG. 4A).

To assess potency and cytokine release of M3167 in comparison to Comparator 1 and Comparator 2, the antigen binding molecules were tested in a Granzyme B release assay essentially described as in Example 6 and an Interferon γ release assay. The concentration of the tested compounds was 0.128 pM to 10 nM. PRAME-positive cancer cells OV56 were prepared in assay medium (DMEM:Ham's F12) and plated at 10,000 cells per well in a volume of 100 μL assay medium. The next day, medium was removed and PBMCs effector cells were plated at 100,000 cells per well in a volume of 100 μL assay medium (RPMI). Varying concentrations of compounds were added to the plated wells in 15 μL assay medium. The final assay medium was made up to 150 μL per well. All reactions were performed in duplicates. The plates were incubated for 24 h at 37° C./5% CO2. Supernatants were collected and Interferon γ release was determined by its quantification in the cell supernatant using the Human IFN gamma Uncoated ELISA Kit from Invitrogen (ThermoFisher Scientific) according to the manufacturer's instructions and the upper limit of quantification (ULOQ) was determined in each assay with an internal standard. M3167 showed higher Granzyme B release than Comparator 2 but less Interferon γ release that Comparator 1, indicating potent cancer cell killing at low cytokine release (FIG. 4B).

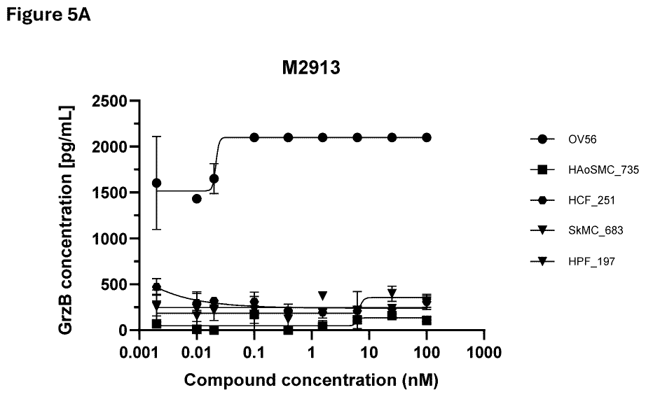

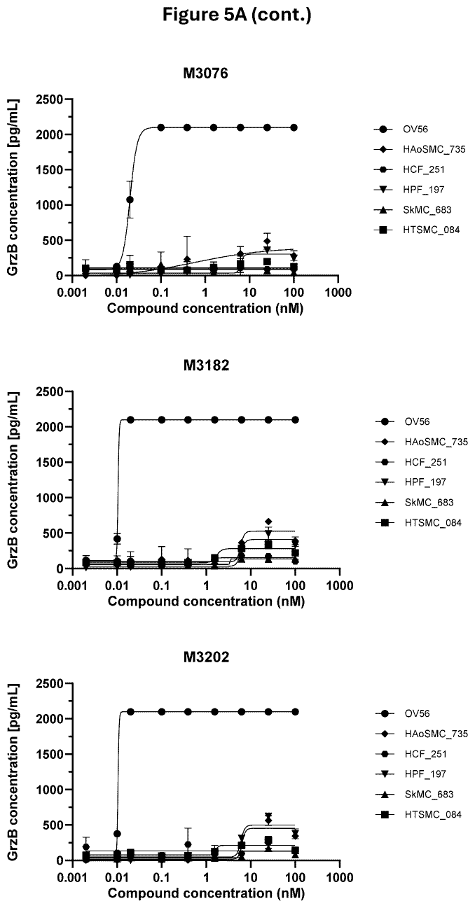

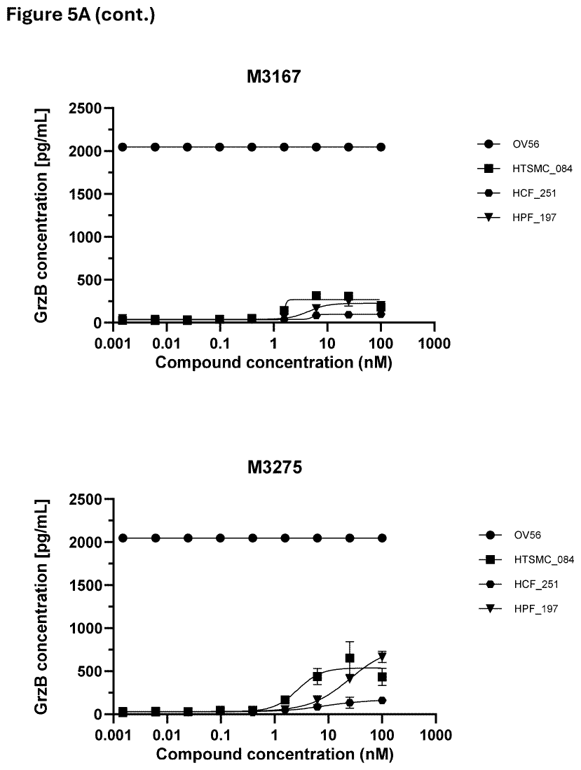

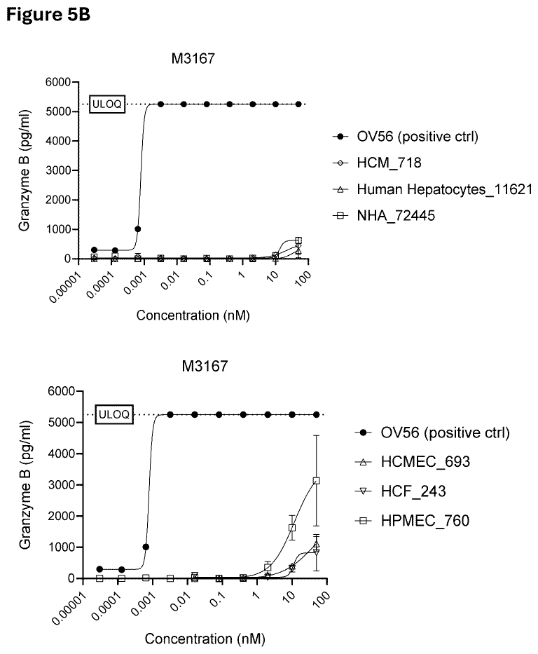

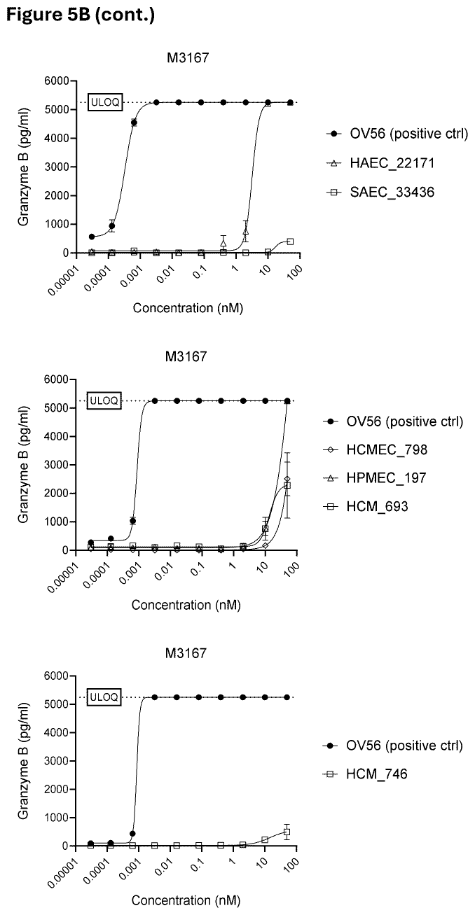

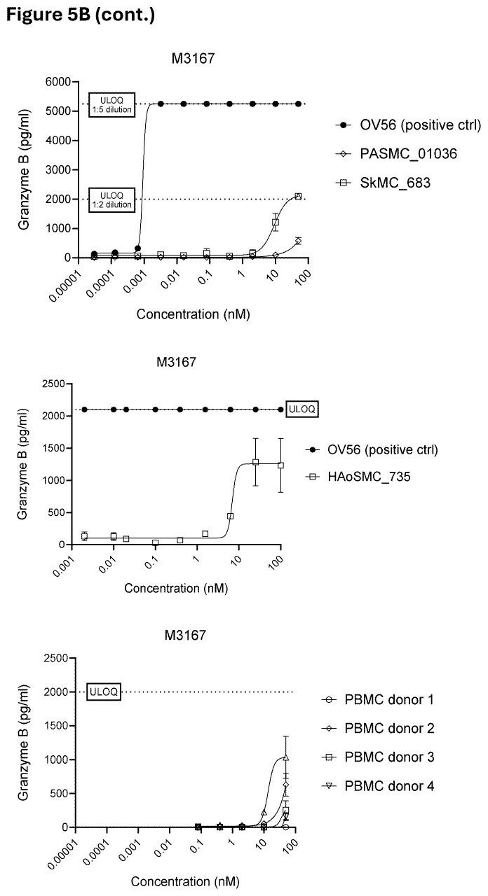

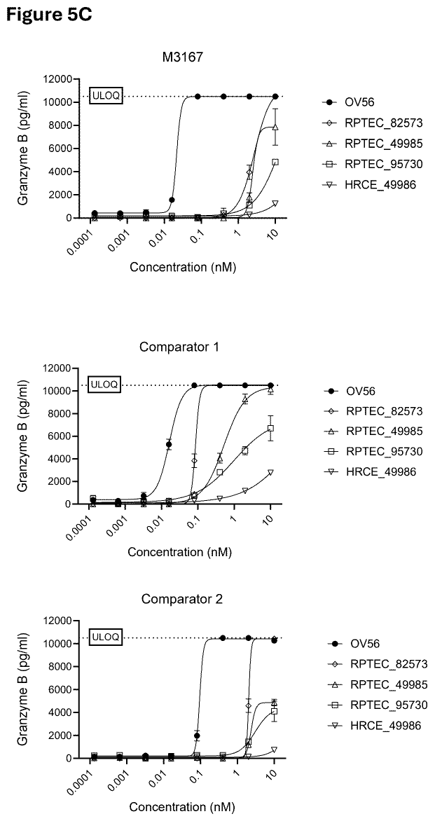

Safety of M2913, M3076, M3182, M3202, M3167 and M3275 was evaluated on human primary cells expressing HLA-A*02:01. Primary cells of various origins spanning essential human tissues, including aortic smooth muscle cells (HAoSMC_735), tracheal smooth muscle cells (HTSMC_084), cardiac fibroblasts (HCF_251), pulmonary fibroblasts (HPF_197) and/or skeletal muscle cells (SkMC_683) were tested as described in Example 6. HLA-A*02:01-positive and PRAME-positive cell line OV56 served as a positive control. All tested molecules showed a safe profile on the tested human primary cells (FIG. 5A). Slight T cell activation responses were only observed at the highest tested compound concentrations and a very good therapeutic window compared to the positive control cancer cell line OV56 was observed for all tested molecules.

To assess a broader safety profile of M3167, an extended panel of HLA-A*02:01-positive human primary cells were tested as described in Example 6 and are summarized in Table 8. HLA-A*02:01-positive and PRAME-positive cell line OV56 served as a positive control. Different concentrations of M3167, starting from 0.026 pM and up to 100 nM were tested and the upper limit of quantification (ULOQ) was determined in each assay with an internal standard. Reactivity on PBMCs from different donors was tested with the compound only. M3167 showed a broad safety profile on all the tested human primary cells (FIG. 5B). A slight Granzyme B release was only measured at the highest tested compound concentrations, showing a very good therapeutic window compared to the positive control cancer cell line OV56.

TABLE 8
HLA-A*02:01- positive primary cells
tested for reactivity with M3167

| Tissue | Cell type | Name |
|---|---|---|
| Heart | Cardiomyocytes | HCM_718, HCM_693, |
| | | HCM_746 |
| | Cardiac microvascular | HCMEC_693, HCMEC_798 |
| | endothelial cells | |
| | Cardiac fibroblasts | HCF_243, HCF_251 |
| Aorta | Aortic endothelial cells | HAEC_22171 |
| | Aortic smooth muscle cells | HAoSMC_735 |
| Lung | Pulmonary microvascular | HPMEC_760, HPMEC_197 |
| | endothelial cells | |
| | Small airway epithelial | SAEC_33436 |
| | cells | |
| | Pulmonary fibroblasts | HPF_197 |
| | Tracheal smooth muscle cells | HTSMC_084 |
| | Pulmonary artery smooth | PASMC_01036 |
| | muscle cells | |
| Brain | Astrocytes | NHA_72445 |
| Liver | Hepatocytes | Human Hepatocytes_11621 |
| Muscle | Skeletal muscle cells | SkMC_683 |
| Blood | Peripheral blood mononuclear | Several donors (PBMCs isolated |
| | cells | in-house from buffy coats) |

Kidneys express low levels of PRAME. To assess the window between on-target/on-tumor and on-target/off-tumor reactivity of M3167 and Comparator 1 and Comparator 2, we tested the Granzyme B release on human primary kidney cells. Granzyme B release was tested essentially as described in Example 6 on HLA-A*02:01-positive renal proximal tubule epithelial cells (RPTEC_82573; RPTEC_49985; RPTEC95730) and/or renal cortical epithelial cells (HRCE_49986). The PRAME-positive cell line OV56 served as a positive control for the on-target/on-tumor reactivity. M3167 showed the safest profile on the tested primary cells compared to Comparator 1 and Comparator 2 with a more than 100-fold window between on-target/on-tumor and on-target/off-tumor reactivity. Comparator 1 was slightly more potent than M3167 but had a very small safety window of less than 10-fold between on-target/on-tumor and on-target/off-tumor reactivity. Comparator 2 was less potent than M3167 and additionally showed a narrower safety window than M3167. The data are summarized in FIG. 5C and demonstrate the strong potency and safety of M3167 alone and in comparison, to comparator antigen binding molecules.

### Example 9—Determination of PRAME Peptide Binding Profile of Humanized and Optimized HLA-A*02:01/PRAMExCD3 Bispecific Antibodies

The molecular recognition profile of monovalent (Fab-scFv) antigen binding molecules M3076 and M3287 for HLA-A*02:01/PRAME binding (equivalent bivalent (Fab-(scFv)₂) antigen binding molecules are M3167 and M3292, respectively) were determined by SPR measurements with HLA-A*02:01 complexes loaded with PRAME peptide or peptides containing a single amino acid substitution to alanine, arginine or aspartic acid. The kinetic parameters for each HLA-A*02:01 complex loaded with a peptide was determined by SPR, according to the previously described method in Example 5. K_{D}-values for each tested peptide loaded on HLA-A*02:01 are shown in Table 9, 10 and 11. For both tested antigen binding proteins, the affinity to the HLA-A*02:01 peptide complex was drastically reduced (K_{D}-values increased) when amino acids on positions 5, 6, 7, 8 and 9 of the peptide were mutated to alanine. A similar recognition pattern was determined with arginine exchange with an additional strong decrease in affinity when position 3 was substituted. The mutation of each amino acid position to aspartic acid showed drastically reduced binding of M3076 to positions 2, 5, 6, 7, 8 and 9 mutants and binding of M3287 to positions 2, 4, 5, 6, 7 and 8 mutants. The results of the peptide scan indicate a broad binding profile of M3076 and M3287 to the peptide covering the majority of the sequence with multiple amino acid positions being crucial for the binding.

TABLE 9
K_{D}-values [nM] of M3076 and M3287 interaction with HLA-A*02:01
loaded with PRAME target peptide and alanine scan peptides.

| | PRAME | S1A | L2A | L3A | Q4A | H5A | L6A | I7A | G8A | L9A |
|---|---|---|---|---|---|---|---|---|---|---|
| M3076 | 0.08 | 0.09 | 0.07 | 0.19 | 0.18 | 3.0 | 15 | 31 | 0.77 | 1.7 |
| M3287 | 0.57 | 1.1 | 0.62 | 1.6 | 1.2 | 16 | 67 | 100 | 9.8 | 18 |

TABLE 10
K_{D}-values [nM] of M3076 and M3287 interaction with HLA-A*02:01
loaded with PRAME target peptide and arginine scan peptides.

| | PRAME | S1R | L2R | L3R | Q4R | H5R | L6R | I7R | G8R | L9R |
|---|---|---|---|---|---|---|---|---|---|---|
| M3076 | 0.08 | 0.08 | 0.04 | 1.0 | 0.08 | 93 | n.b. | 18 | n.b. | 1.4 |
| M3287 | 0.57 | 0.57 | 0.31 | 9.2 | 0.49 | n.b. | n.b. | 83 | n.b. | 6.1 |

N.b.: no binding detected.

TABLE 11
K_{D}-values [nM] of M3076 and M3287 interaction with HLA-A*02:01
loaded with PRAME target peptide and aspartic acid scan peptides.

| | PRAME | S1D | L2D | L3D | Q4D | H5D | L6D | I7D | G8D | L9D |
|---|---|---|---|---|---|---|---|---|---|---|
| M3076 | 0.08 | 0.06 | n.b. | 0.09 | 0.34 | 0.44 | n.b. | n.b. | n.b. | 0.83 |
| M3287 | 0.57 | 0.53 | n.b. | 0.63 | 3.4 | 4.6 | n.b. | n.b. | n.b. | 2.7 |

N.b.: no binding detected.

### Example 10—Stability Characterization of Humanized and Optimized HLA-A*02:01/PRAMExCD3 Bispecific Antibodies at Simulated In Vivo Conditions

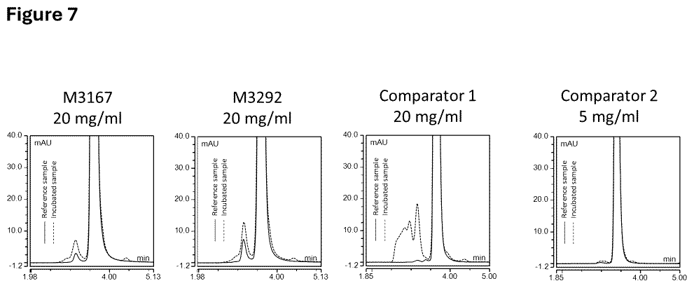

M3167, M3292 and the two comparator antigen binding molecules Comparator 1 and Comparator 2 were tested for stability at high concentrations of up to 20 mg/ml during an incubation of seven days at 34° C. in a carbonate-based artificial subcutaneous fluid buffer (pH 7.4), as essentially described in Table 3 (1×Aqix buffer) in J. Schuster et al, Journal of Pharmaceutical Science, 110 (2021), 2386-2394. Stability was assessed by SEC quantification of the target protein monomer, and the corresponding data is presented in FIG. 7. High concentrations of 20 mg/ml were reached for M3167, M3292 and Comparator 1, whereas Comparator 2 was only soluble up to 5 mg/mL. M3167, M3292 and Comparator 2 showed good stability in artificial subcutaneous fluid buffer (pH 7.4) at 34° C. for 7 days showing <3% target protein oligomerization at 20 mg/mL and 5 mg/ml, respectively. Measured at the same conditions as M3167 and M3292, Comparator 1 showed a relatively low stability with 11.3% target protein oligomerization after incubation for seven days. The data demonstrate the superior solubility and stability of M3167 and M3292 compared to the comparator antigen binding molecules.

### Example 11—Determination of HLA-A*02:01/PRAME Cell Copy Numbers Presented on Lung Adenocarcinoma Biopsies

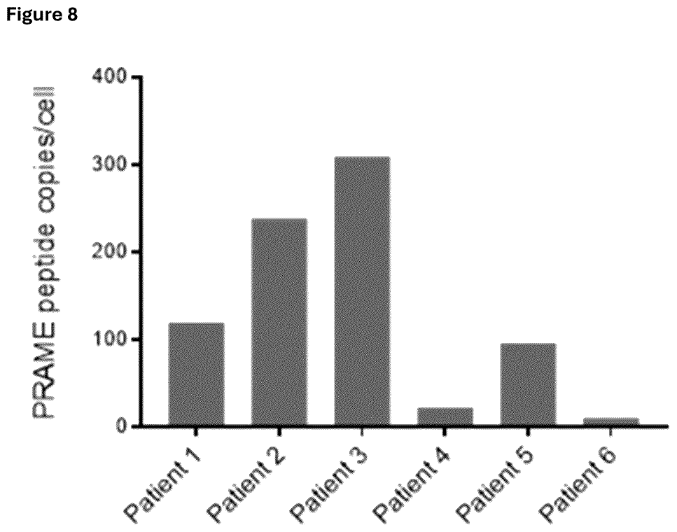

HLA-A*02:01/PRAME presentation was analyzed on twelve tumor biopsies from lung adenocarcinoma patients. To determine the copy number per cell, HLA complexes were purified from the HLA-A*02:01-positive biopsies. The samples were lysed and HLA class I complexes were purified with monoclonal anti-HLA class I (clone W6/32) antibody-coupled resin. Peptides were eluted from HLA complexes and further purified with C18 Macro SpinColums and eluted in 30% acetonitrile, 0.1% trifluoroacetic acid. Heavy-labeled PRAME peptides (SEQ ID NO: 139) were spiked to each of the HLA-peptide preparations and subjected to MS analysis. The PRAME peptide (SEQ ID NO: 139) could be identified in 50% of the samples with copy numbers between 10 and 309 per cell (FIG. 8).

### Example 12—Analysis of PRAME RNA Expression Levels for the Determination of Tumor Indications and Potential Patient Populations

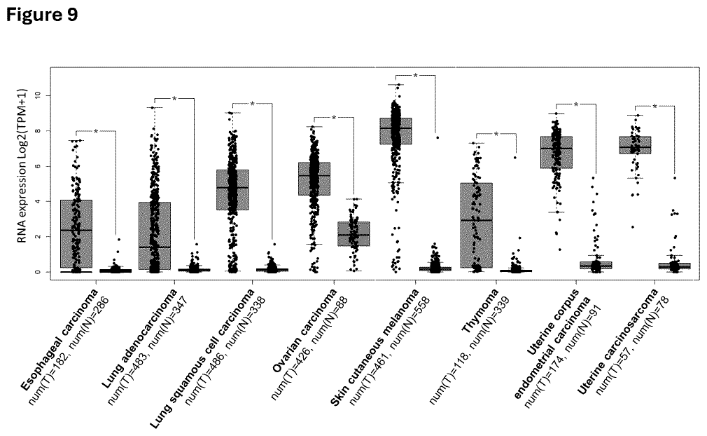

A differential gene expression analysis of PRAME was performed by using the web-based tool GEPIA (Gene Expression Profiling Interactive Analysis), that combines RNA expression levels from the TCGA and the GTEx database (Tang et al., Nucleic Acids Res., 2017; 45(W1):W98-W102). PRAME RNA expression levels of various tumors were analyzed and revealed significantly increased PRAME expression in esophageal carcinoma, lung adenocarcinoma, lung squamous cell carcinoma, ovarian carcinoma, skin cutaneous melanoma, thymoma, uterine corpus endometrial carcinoma and/or uterine carcinosarcoma when compared to corresponding healthy tissues (FIG. 9). The analysis indicates the high unmet need that can be addressed by targeting PRAME positive tumors.

1. An antigen binding protein which specifically binds to a major histocompatibility complex (MHC)-displayed SLLQHLIGL (SEQ ID NO: 139), wherein the antigen binding protein comprises:
(a) an antibody heavy chain variable (VH) domain comprising an HCDR1 amino acid sequence of STYGVS (SEQ ID NO: 25),
an HCDR2 amino acid sequence of YIDPVYGSTX₁YAX₂×₃VX₄G (SEQ ID NO: 26), wherein
X₁ is Yor H, X₂ is S or D, X₃ is W or S, and X₄ is N or K, and
an HCDR3 amino acid sequence of DLYAGSSGYYX₅IYSL (SEQ ID NO: 27), wherein X₅ is M or V; and
(b) an antibody light chain variable (VL) domain comprising
an LCDR1 amino acid sequence of X₆SSQSVYNNLLG (SEQ ID NO: 28), wherein X₆ is Q or R,
an LCDR2 amino acid sequence of SASTX₇AS (SEQ ID NO: 29), wherein X₇ is L or R, and
an LCDR3 amino acid sequence of QGTYYX₈GDWYYP (SEQ ID NO: 30), wherein X₈ is N or T.

2. The antigen binding protein of claim 1, wherein the antigen binding protein has an affinity (KD) to an MHC-displayed SLLQHLIGL (SEQ ID NO: 139) of at least about 5 nM or stronger, optionally, wherein the MHC is of HLA supertype A*02, in particular HLA-A*02:01.

3-4. (canceled)

5. The antigen binding protein of claim 1, wherein
the antigen binding protein comprises
(a1) an antibody heavy chain variable (VH) domain comprising an HCDR1 amino acid sequence of STYGVS (SEQ ID NO: 37), an HCDR2 amino acid sequence of YIDPVYGSTHYADSVKG (SEQ ID NO: 38), and an HCDR3 amino acid sequence of DLYAGSSGYYMIYSL (SEQ ID NO: 39); and (b1) an antibody light chain variable (VL) domain comprising an LCDR1 amino acid sequence of RSSQSVYNNLLG (SEQ ID NO: 40), an LCDR2 amino acid sequence of SASTLAS (SEQ ID NO: 41), and an LCDR3 amino acid sequence of QGTYYNGDWYYP (SEQ ID NO: 42);
(a2) an antibody heavy chain variable (VH) domain comprising an HCDR1 amino acid sequence of STYGVS (SEQ ID NO: 31), an HCDR2 amino acid sequence of YIDPVYGSTYYASWVNG (SEQ ID NO: 32), and an HCDR3 amino acid sequence of DLYAGSSGYYMIYSL (SEQ ID NO: 33); and (b2) an antibody light chain variable (VL) domain comprising an LCDR1 amino acid sequence of QSSQSVYNNLLG (SEQ ID NO: 34), an LCDR2 amino acid sequence of SASTLAS (SEQ ID NO: 35), and an LCDR3 amino acid sequence of QGTYYNGDWYYP (SEQ ID NO: 36);
(a3) an antibody heavy chain variable (VH) domain comprising an HCDR1 amino acid sequence of STYGVS (SEQ ID NO: 43), an HCDR2 amino acid sequence of YIDPVYGSTHYADSVKG (SEQ ID NO: 44), and an HCDR3 amino acid sequence of DLYAGSSGYYMIYSL (SEQ ID NO: 45); and (b3) an antibody light chain variable (VL) domain comprising an LCDR1 amino acid sequence of RSSQSVYNNLLG (SEQ ID NO: 46), an LCDR2 amino acid sequence of SASTLAS (SEQ ID NO: 47), and an LCDR3 amino acid sequence of QGTYYTGDWYYP (SEQ ID NO: 48);
(a4) an antibody heavy chain variable (VH) domain comprising an HCDR1 amino acid sequence of STYGVS (SEQ ID NO: 49), an HCDR2 amino acid sequence of YIDPVYGSTHYADSVKG (SEQ ID NO: 50), and an HCDR3 amino acid sequence of DLYAGSSGYYVIYSL (SEQ ID NO: 51); and (b4) an antibody light chain variable (VL) domain comprising an LCDR1 amino acid sequence of RSSQSVYNNLLG (SEQ ID NO: 52), an LCDR2 amino acid sequence of SASTLAS (SEQ ID NO: 53), and an LCDR3 amino acid sequence of QGTYYTGDWYYP (SEQ ID NO: 54); or
(a5) an antibody heavy chain variable (VH) domain comprising an HCDR1 amino acid sequence of STYGVS (SEQ ID NO: 55), an HCDR2 amino acid sequence of YIDPVYGSTHYADSVKG (SEQ ID NO: 56), and an HCDR3 amino acid sequence of DLYAGSSGYYMIYSL (SEQ ID NO: 57); and (b5) an antibody light chain variable (VL) domain comprising an LCDR1 amino acid sequence of RSSQSVYNNLLG (SEQ ID NO: 58), an LCDR2 amino acid sequence of SASTRAS (SEQ ID NO: 59), and an LCDR3 amino acid sequence of QGTYYTGDWYYP (SEQ ID NO: 60).

6. The antigen binding protein of claim 1, wherein:
the antibody heavy chain variable (VH) domain comprises one or more framework sequences HFR1, HFR2, HFR3 and/or HFR4 selected from the group consisting of SEQ ID NO: 61, SEQ ID NO: 69, SEQ ID NO: 62, SEQ ID NO: 63 and/or SEQ ID NO: 64, or respective variants thereof comprising 1, 2, 3, 4, 5, or 6 substitutions;
the antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 67, SEQ ID NO: 70 and/or SEQ ID NO: 68 or respective variants thereof comprising 1, 2, 3, 4, 5, or 6 substitutions; optionally wherein
the antigen binding protein comprises:
(1) an antibody heavy chain variable (VH) domain comprising one or more framework sequences HFR1, HFR2, HFR3 and/or HFR4 selected from the group consisting of SEQ ID NO: 204, SEQ ID NO: 62, SEQ ID NO: 63 and/or SEQ ID NO: 64 or respective variants thereof comprising 1, 2, 3, 4, 5, or 6 substitutions; and an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 67 and/or SEQ ID NO: 68 or respective variants thereof comprising 1, 2, 3, 4, 5, or 6 substitutions;
(2) an antibody heavy chain variable (VH) domain comprising one or more framework sequences HFR1, HFR2, HFR3 and/or HFR4 selected from the group consisting of SEQ ID NO: 61, SEQ ID NO: 62, SEQ ID NO: 63 and/or SEQ ID NO: 64 or respective variants thereof comprising 1, 2, 3, 4, 5, or 6 substitutions; and an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 70 and/or SEQ ID NO: 68 or respective variants thereof comprising 1, 2, 3, 4, 5, or 6 substitutions;
(3) an antibody heavy chain variable (VH) domain comprising one or more framework sequences HFR1, HFR2, HFR3 and/or HFR4 selected from the group consisting of SEQ ID NO: 69, SEQ ID NO: 62, SEQ ID NO: 63 and/or SEQ ID NO: 64 or respective variants thereof comprising 1, 2, 3, 4, 5, or 6 substitutions; and an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 67 and/or SEQ ID NO: 68 or respective variants thereof comprising 1, 2, 3, 4, 5, or 6 substitutions; or
(4) an antibody heavy chain variable (VH) domain comprising one or more framework sequences HFR1, HFR2, HFR3 and/or HFR4 selected from the group consisting of SEQ ID NO: 69, SEQ ID NO: 62, SEQ ID NO: 63 and/or SEQ ID NO: 64 or respective variants thereof comprising 1, 2, 3, 4, 5, or 6 substitutions; and an antibody light chain variable (VL) domain comprising one or more framework sequences LFR1, LFR2, LFR3 and/or LFR4 selected from the group consisting of SEQ ID NO: 65, SEQ ID NO: 66, SEQ ID NO: 70 and/or SEQ ID NO: 68 or respective variants thereof comprising 1, 2, 3, 4, 5, or 6 substitutions.

7-8. (canceled)

9. The antigen binding protein of claim 1, comprising:
(1) an antibody heavy chain variable (VH) domain comprising the amino acid sequence of SEQ ID NO: 75 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 76 or a variant thereof that is at least about 75%, 78%, 79%, 80%, 81%, 83%, 85%, 90%, 95%, 96%, 97%, 98% or 99% identical thereto;
(2) an antibody heavy chain variable (VH) domain comprising the amino acid sequence of SEQ ID NO: 71 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 72 or a variant thereof that is at least about 75%, 78%, 79%, 80%, 81%, 83%, 85%, 90%, 95%, 96%, 97%, 98% or 99% identical thereto;
(3) an antibody heavy chain variable (VH) domain comprising the amino acid sequence of SEQ ID NO: 73 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 74 or a variant thereof that is at least 75%, 78%, 79%, 80%, 81%, 83%, 85%, 90%, 95%, 96%, 97%, 98% or 99% identical thereto;
(4) an antibody heavy chain variable (VH) domain comprising the amino acid sequence of SEQ ID NO: 77 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 78 or a variant thereof that is at least about 75%, 78%, 79%, 80%, 81%, 83%, 85%, 90%, 95%, 96%, 97%, 98% or 99% identical thereto;
(5) an antibody heavy chain variable (VH) domain comprising the amino acid sequence of SEQ ID NO: 79 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 80 or a variant thereof that is at least about 75%, 78%, 79%, 80%, 81%, 83%, 85%, 90%, 95%, 96%, 97%, 98% or 99% identical thereto;
(6) an antibody heavy chain variable (VH) domain comprising the amino acid sequence of SEQ ID NO: 81 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 82 or a variant thereof that is at least about 75%, 78%, 79%, 80%, 81%, 83%, 85%, 90%, 95%, 96%, 97%, 98% or 99% identical thereto;
(7) an antibody heavy chain variable (VH) domain comprising the amino acid sequence of SEQ ID NO: 83 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 84 or a variant thereof that is at least about 75%, 78%, 79%, 80%, 81%, 83%, 85%, 90%, 95%, 96%, 97%, 98% or 99% identical thereto;
(8) an antibody heavy chain variable (VH) domain comprising the amino acid sequence of SEQ ID NO: 85 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 86 or a variant thereof that is at least about 75%, 78%, 79%, 80%, 81%, 83%, 85%, 90%, 95%, 96%, 97%, 98% or 99% identical thereto;
(9) an antibody heavy chain variable (VH) domain comprising the amino acid sequence of SEQ ID NO: 87 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 88 or a variant thereof that is at least about 75%, 78%, 79%, 80%, 81%, 83%, 85%, 90%, 95%, 96%, 97%, 98% or 99% identical thereto;
(10) an antibody heavy chain variable (VH) domain comprising the amino acid sequence of SEQ ID NO: 89 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 90 or a variant thereof that is at least about 75%, 78%, 79%, 80%, 81%, 83%, 85%, 90%, 95%, 96%, 97%, 98% or 99% identical thereto; or
(11) an antibody heavy chain variable (VH) domain comprising the amino acid sequence of SEQ ID NO: 91 or a variant thereof that is at least about 75%, 78%, 79%, 80%, 81%, 83%, 85%, 90%, 95%, 96%, 97%, 98% or 99% identical thereto; and an antibody light chain variable (VL) domain comprising the amino acid sequence of SEQ ID NO: 92 or a variant thereof that is at least about 75%, 78%, 79%, 80%, 81%, 83%, 85%, 90%, 95%, 96%, 97%, 98% or 99% identical thereto.

10-13. (canceled)

14. The antigen binding protein of claim 1, being or comprising a scFv with an amino acid sequence that is at least about 79%, 80%, 85%, 90%, 95%, 96%, 97%, 98%, 99% or 100% identical to the amino acid sequence of SEQ ID NO: 95, SEQ ID NO: 93, SEQ ID NO: 94, SEQ ID NO: 96, SEQ ID NO: 97, SEQ ID NO: 98, SEQ ID NO: 99, SEQ ID NO: 100, SEQ ID NO: 101, SEQ ID NO: 102 or SEQ ID NO: 103.

15-22. (canceled)

23. A chimeric antigen receptor (CAR) comprising the antigen binding protein of claim 1.

24. An immune cell expressing the CAR of claim 23, in particular wherein the immune cell is a T cell.

25. An antibody drug conjugate (ADC) comprising the antigen binding protein of claim 1.

26. A multispecific antigen binding protein comprising the antigen binding protein of claim 1, optionally being bispecific or trispecific.

27-32. (canceled)

33. The multispecific antigen binding protein of claim 26, comprising an immune cell binding domain, optionally wherein the immune cell binding domain targets CD3, optionally wherein the CD3 binding domain is a Fab consisting of a HC and an FC domain, and the HC domain comprises an amino acid sequence that is at least about 90%, 95%, 96%, 97%, 98%, 99% or 100% identical to the amino acid sequence of SEQ ID NO: 104, and the VL domain comprises an amino acid sequence that is at least about 90%, 95%, 96%, 97%, 98%, 99% or 100% identical to the amino acid sequence of SEQ ID NO: 105.

34. (canceled)

35. The multispecific antigen binding protein of claim 26, being a Fab-scFv, comprising or consisting of
(i) the scFv of SEQ ID NO: 95, being linked to the C-terminus of the HC of SEQ ID NO: 104; and a LC of SEQ ID NO: 105;
(ii) the scFv of SEQ ID NO: 93, being linked to the C-terminus of the HC of SEQ ID NO: 104; and a LC of SEQ ID NO: 105;
(iii) (i) the scFv of SEQ ID NO: 94, being linked to the C-terminus of the HC of SEQ ID NO: 104; and a LC of SEQ ID NO: 105;
(iv) the scFv of SEQ ID NO: 96, being linked to the C-terminus of the HC of SEQ ID NO: 104; and a LC of SEQ ID NO: 105;
(v) the scFv of SEQ ID NO: 97, being linked to the C-terminus of the HC of SEQ ID NO: 104; and a LC of SEQ ID NO: 105;
(vi) the scFv of SEQ ID NO: 98, being linked to the C-terminus of the HC of SEQ ID NO: 104; and a LC of SEQ ID NO: 105;
(vii) the scFv of SEQ ID NO: 99, being linked to the C-terminus of the HC of SEQ ID NO: 104; and a LC of SEQ ID NO: 105;
(viii) the scFv of SEQ ID NO: 100, being linked to the C-terminus of the HC of SEQ ID NO: 104; and a LC of SEQ ID NO: 105;
(ix) the scFv of SEQ ID NO: 101, being linked to the C-terminus of the HC of SEQ ID NO: 104; and a LC of SEQ ID NO: 105;
(x) the scFv of SEQ ID NO: 102, being linked to the C-terminus of the HC of SEQ ID NO: 104; and a LC of SEQ ID NO: 105; or
(xi) the scFv of SEQ ID NO: 103, being linked to the C-terminus of the HC of SEQ ID NO: 104; and a LC of SEQ ID NO: 105;
or variants of said sequences that are at least about 90%, 95%, 96%, 97%, 98%, 99% or 100% identical to the amino acid sequences, optionally wherein the multispecific antigen binding protein comprises or consists of
(i) the HC of SEQ ID NO: 108; and the LC of SEQ ID NO: 105;
(ii) the HC of SEQ ID NO: 106; and the LC of SEQ ID NO: 105;
(iii) the HC of SEQ ID NO: 107; and the LC of SEQ ID NO: 105;
(iv) the HC of SEQ ID NO: 109; and the LC of SEQ ID NO: 105;
(v) the HC of SEQ ID NO: 110; and the LC of SEQ ID NO: 105;
(vi) the HC of SEQ ID NO: 111; and the LC of SEQ ID NO: 105;
(vii) the HC of SEQ ID NO: 112; and the LC of SEQ ID NO: 105;
(viii) the HC of SEQ ID NO: 113; and the LC of SEQ ID NO: 105;
(ix) the HC of SEQ ID NO: 114; and the LC of SEQ ID NO: 105;
(x) the HC of SEQ ID NO: 115; and the LC of SEQ ID NO: 105; or
the HC of SEQ ID NO: 116; and the LC of SEQ ID NO: 105.

36-58. (canceled)

59. The multispecific antigen binding protein of claim 26, being a bispecific Fab-(scFv)₂ comprising
(a) a CD3 targeting Fab domain comprising the HCDRs of SEQ ID NOs: 140, 141 and 142 and/or the LCDRs of SEQ ID NO.: 143, 144 and 145; and
(b) two antigen binding scFvs specifically binding to MHC-displayed SLLQHLIGL (SEQ ID NO: 139), each scFv comprising:
(i) an antibody heavy chain variable (VH) domain comprising the HCDR amino acid sequences of SEQ ID NOs: 25, 26, and 27; and
(ii) an antibody light chain variable (VL) domain comprising the LCDR amino acid sequences of SEQ ID NOs.: 28, 29, and 30.

60. (canceled)

61. The bispecific Fab-(scFv)₂ of claim 59, wherein the antigen binding scFv comprises:
(1) (i) an antibody heavy chain variable (VH) domain that is at least about 90%, 95%, 96%, 97%, 98%, 99% or 100% identical to SEQ ID NO: 75; and (ii) an antibody light chain variable (VL) domain that is at least about 90%, 95%, 96%, 97%, 98%, 99% or 100% identical to SEQ ID NO: 76;
(2) (i) an antibody heavy chain variable (VH) domain that is at least about 90%, 95%, 96%, 97%, 98%, 99% or 100% identical to SEQ ID NO: 71; and (ii) an antibody light chain variable (VL) domain that is at least about 90%, 95%, 96%, 97%, 98%, 99% or 100% identical to SEQ ID NO: 72;
(3) (i) an antibody heavy chain variable (VH) domain that is at least about 90%, 95%, 96%, 97%, 98%, 99% or 100% identical to SEQ ID NO: 73; and (ii) an antibody light chain variable (VL) domain that is at least about 90%, 95%, 96%, 97%, 98%, 99% or 100% identical to SEQ ID NO: 74;
(4) (i) an antibody heavy chain variable (VH) domain that is at least about 90%, 95%, 96%, 97%, 98%, 99% or 100% identical to SEQ ID NO: 77; and (ii) an antibody light chain variable (VL) domain that is at least about 90%, 95%, 96%, 97%, 98%, 99% or 100% identical to SEQ ID NO: 78;
(5) (i) an antibody heavy chain variable (VH) domain that is at least about 90%, 95%, 96%, 97%, 98%, 99% or 100% identical to SEQ ID NO: 79; and (ii) an antibody light chain variable (VL) domain that is at least about 90%, 95%, 96%, 97%, 98%, 99% or 100% identical to SEQ ID NO: 80;
(6) (i) an antibody heavy chain variable (VH) domain that is at least about 90%, 95%, 96%, 97%, 98%, 99% or 100% identical to SEQ ID NO: 81; and (ii) an antibody light chain variable (VL) domain that is at least about 90%, 95%, 96%, 97%, 98%, 99% or 100% identical to SEQ ID NO: 82;
(7) (i) an antibody heavy chain variable (VH) domain that is at least about 90%, 95%, 96%, 97%, 98%, 99% or 100% identical to SEQ ID NO: 83; and (ii) an antibody light chain variable (VL) domain that is at least about 90%, 95%, 96%, 97%, 98%, 99% or 100% identical to SEQ ID NO: 84;
(8) (i) an antibody heavy chain variable (VH) domain that is at least about 90%, 95%, 96%, 97%, 98%, 99% or 100% identical to SEQ ID NO: 85; and (ii) an antibody light chain variable (VL) domain that is at least about 90%, 95%, 96%, 97%, 98%, 99% or 100% identical to SEQ ID NO: 86;
(9) (i) an antibody heavy chain variable (VH) domain that is at least about 90%, 95%, 96%, 97%, 98%, 99% or 100% identical to SEQ ID NO: 87; and (ii) an antibody light chain variable (VL) domain that is at least about 90%, 95%, 96%, 97%, 98%, 99% or 100% identical to SEQ ID NO: 88;
(10) (i) an antibody heavy chain variable (VH) domain that is at least about 90%, 95%, 96%, 97%, 98%, 99% or 100% identical to SEQ ID NO: 89; and (ii) an antibody light chain variable (VL) domain that is at least about 90%, 95%, 96%, 97%, 98%, 99% or 100% identical to SEQ ID NO: 90; or
(11) (i) an antibody heavy chain variable (VH) domain that is at least about 90%, 95%, 96%, 97%, 98%, 99% or 100% identical to SEQ ID NO: 91; and (ii) an antibody light chain variable (VL) domain that is at least about 90%, 95%, 96%, 97%, 98%, 99% or 100% identical to SEQ ID NO: 92.

62. The multispecific antigen binding protein of claim 26, being a Fab-(scFv)₂, comprising or consisting of
(1) (i) a heavy chain (HC) that is at least about 90%, 95%, 96%, 97%, 98%, 99% or 100% identical to SEQ ID NO: 121; and (ii) a light chain (LC) that is at least about 90%, 95%, 96%, 97%, 98%, 99% or 100% identical to SEQ ID NO: 122;
(2) (i) a heavy chain (HC) that is at least about 90%, 95%, 96%, 97%, 98%, 99% or 100% identical to SEQ ID NO: 117; and (ii) a light chain (LC) that is at least about 90%, 95%, 96%, 97%, 98%, 99% or 100% identical to SEQ ID NO: 118;
(3) (i) a heavy chain (HC) that is at least about 90%, 95%, 96%, 97%, 98%, 99% or 100% identical to SEQ ID NO: 119; and (ii) a light chain (LC) that is at least about 90%, 95%, 96%, 97%, 98%, 99% or 100% identical to SEQ ID NO: 120;
(4) (i) a heavy chain (HC) that is at least about 90%, 95%, 96%, 97%, 98%, 99% or 100% identical to SEQ ID NO: 123; and (ii) a light chain (LC) that is at least about 90%, 95%, 96%, 97%, 98%, 99% or 100% identical to SEQ ID NO: 124;
(5) (i) a heavy chain (HC) that is at least about 90%, 95%, 96%, 97%, 98%, 99% or 100% identical to SEQ ID NO: 125; and (ii) a light chain (LC) that is at least about 90%, 95%, 96%, 97%, 98%, 99% or 100% identical to SEQ ID NO: 126;
(6) (i) a heavy chain (HC) that is at least about 90%, 95%, 96%, 97%, 98%, 99% or 100% identical to SEQ ID NO: 127; and (ii) a light chain (LC) that is at least about 90%, 95%, 96%, 97%, 98%, 99% or 100% identical to SEQ ID NO: 128;
(7) (i) a heavy chain (HC) that is at least about 90%, 95%, 96%, 97%, 98%, 99% or 100% identical to SEQ ID NO: 129; and (ii) a light chain (LC) that is at least about 90%, 95%, 96%, 97%, 98%, 99% or 100% identical to SEQ ID NO: 130;
(8) (i) a heavy chain (HC) that is at least about 90%, 95%, 96%, 97%, 98%, 99% or 100% identical to SEQ ID NO: 131; and (ii) a light chain (LC) that is at least about 90%, 95%, 96%, 97%, 98%, 99% or 100% identical to SEQ ID NO: 132;
(9) (i) a heavy chain (HC) that is at least about 90%, 95%, 96%, 97%, 98%, 99% or 100% identical to SEQ ID NO: 133; and (ii) a light chain (LC) that is at least about 90%, 95%, 96%, 97%, 98%, 99% or 100% identical to SEQ ID NO: 134;
(10) (i) a heavy chain (HC) that is at least about 90%, 95%, 96%, 97%, 98%, 99% or 100% identical to SEQ ID NO: 135; and (ii) a light chain (LC) that is at least about 90%, 95%, 96%, 97%, 98%, 99% or 100% identical to SEQ ID NO: 136; or
(11) (i) a heavy chain (HC) that is at least about 90%, 95%, 96%, 97%, 98%, 99% or 100% identical to SEQ ID NO: 137; and (ii) a light chain (LC) that is at least about 90%, 95%, 96%, 97%, 98%, 99% or 100% identical to SEQ ID NO: 138.

63. The multispecific antigen binding protein of claim 37, wherein the light chain and/or heavy chain comprises an N-terminal and/or C-terminal truncation of 1, 2, 3, 4, or 5 amino acids, optionally wherein the light chain comprises an N-terminal truncation of 1 or 2 amino acids, and/or
wherein the multispecific antigen binding protein comprises a pyroglutamate (pE) at position 1 instead of glutamine (Q) or glutamate (E) of the light chain and/or heavy chain, optionally comprising a pyroglutamate (pE) at position 1 instead of glutamine (Q) or glutamate (E) of the light chain.

64-66. (canceled)

67. A nucleic acid encoding the antigen binding protein of claim 1.

68. A vector comprising the nucleic acid of claim 67.

69. A host cell population comprising the vector of claim 68.

70. A method of manufacturing the antigen binding protein of claim 1, comprising the steps of:
(i) cultivating a host cell population under conditions allowing expression of the antigen binding protein or the multispecific antigen binding protein;
(ii) recovering the antigen binding protein or the multispecific antigen binding protein; and optionally
(iii) further purifying and/or modifying and/or formulating the antigen binding protein or the multispecific antigen binding protein.

71-76. (canceled)

77. A pharmaceutical composition comprising the antigen binding protein of claim 1, and a pharmaceutically acceptable buffer.

78-80. (canceled)

81. A method of treating a cancer expressing MHC-displayed SLLQHLIGL (SEQ ID NO: 139) in a patient in need thereof comprising administering to the patient a therapeutically effective amount of the antigen binding protein of claim 1, optionally wherein the cancer is ovarian cancer, NSCLC, melanoma or synovial sarcoma.

82. (canceled)

83. A nucleic acid encoding the the multispecific antigen binding protein of claim 26.

84. A pharmaceutical composition comprising the multispecific antigen binding protein of claim 26, and a pharmaceutically acceptable buffer.

85. A method of treating a cancer expressing NMC-displayed SLLQHLIGL (SEQ ID NO: 139) in a patient in need thereof comprising administering to the patient a therapeutically effective amount of the multispecific antigen binding protein of claim 26, optionally wherein the cancer is ovarian cancer, NSCLC, melanoma or synovial sarcoma.
